(19) United States
(12) Patent Application Publication
(10) Pub. No.: US 2005/0027007 A1
(43) Pub. Date: Feb. 3, 2005

(54) Allylamides useful in the treatment of alzheimer's disease

(76) Inventor: Roy Hom, San Francisco, CA (US)

Correspondence Address: MCDONNELL BOEHNEN HULBERT & BERGHOFF LLP, 300 S. WACKER DRIVE, 32ND FLOOR, CHICAGO, IL, 60606, US

(21) Appl. No.: 10/491,841

(22) PCT Filed: Oct. 4, 2002
(86) PCT No.: PCT/US02/32010

Related U.S. Application Data
(60) Provisional application No. 60/327,243, filed on Oct. 5, 2001.

(51) Int. Cl. A61K 31/195; A61K 31/16

(52) U.S. Cl. 514/563; 514/616

(57) ABSTRACT

Disclosed are methods for treating Alzheimer's disease, and other diseases, and/or inhibiting beta-secretase enzyme, and/or inhibiting deposition of A beta peptide in a mammal, by use of compounds of formula (I) where R₁, R₂, R₃, B, J¹, J², X and Z are as defined herein.

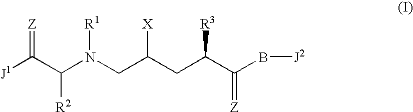

This application claims priority to U.S. Provisional Patent Application No. 60/327,243, filed on Oct. 5, 2001.

### FIELD OF THE INVENTION

The present invention relates to the treatment of Alzheimer's disease and other similar diseases, and more specifically to the use of compounds that inhibit beta-secretase, an enzyme that cleaves amyloid precursor protein to produce A beta peptide, a major component of the amyloid plaques found in the brains of Alzheimer's sufferers, in such methods.

### BACKGROUND OF THE INVENTION

Alzheimer's disease (AD) is a progressive degenerative disease of the brain primarily associated with aging. Clinical presentation of AD is characterized by loss of memory, cognition, reasoning, judgment, and orientation. As the disease progresses, motor, sensory, and linguistic abilities are also affected until there is global impairment of multiple cognitive functions. These cognitive losses occur gradually, but typically lead to severe impairment and eventual death in the range of four to twelve years.

Alzheimer's disease is characterized by two major pathologic observations in the brain: neurofibrillary tangles and beta amyloid (or neuritic) plaques, comprised predominantly of an aggregate of a peptide fragment know as A beta. Individuals with AD exhibit characteristic beta-amyloid deposits in the brain (beta amyloid plaques) and in cerebral blood vessels (beta amyloid angiopathy) as well as neurofibrillary tangles. Neurofibrillary tangles occur not only in Alzheimer's disease but also in other dementia-inducing disorders. On autopsy, large numbers of these lesions are generally found in areas of the human brain important for memory and cognition.

Smaller numbers of these lesions in a more restricted anatomical distribution are found in the brains of most aged humans who do not have clinical AD. Amyloidogenic plaques and vascular amyloid angiopathy also characterize the brains of individuals with Trisomy 21 (Down's Syndrome), Hereditary Cerebral Hemorrhage with Amyloidosis of the Dutch-Type (HCHWA-D), and other neurodegenerative disorders. Beta-amyloid is a defining feature of AD, now believed to be a causative precursor or factor in the development of disease. Deposition of A beta in areas of the brain responsible for cognitive activities is a major factor in the development of AD. Beta-amyloid plaques are predominantly composed of amyloid beta peptide (A beta, also sometimes designated betaA4). A beta peptide is derived by proteolysis of the amyloid precursor protein (APP) and is comprised of 39-42 amino acids. Several proteases called secretases are involved in the processing of APP.

Cleavage of APP at the N-terminus of the A beta peptide by beta-secretase and at the C-terminus by one or more gamma-secretases constitutes the beta-amyloidogenic pathway, i.e. the pathway by which A beta is formed. Cleavage of APP by alpha-secretase produces alpha-sAPP, a secreted form of APP that does not result in beta-amyloid plaque formation. This alternate pathway precludes the formation of A beta peptide. A description of the proteolytic processing fragments of APP is found, for example, in U.S. Pat. Nos. 5,441,870; 5,721,130; and 5,942,400.

An aspartyl protease has been identified as the enzyme responsible for processing of APP at the beta-secretase cleavage-site. The beta-secretase enzyme has been disclosed using varied nomenclature, including BACE, Asp, and Memapsin. See, for example, Sindha et al., 1999, Nature 402:537-554 (p501) and published PCT application WO00/17369.

Several lines of evidence indicate that progressive cerebral deposition of beta-amyloid peptide (A beta) plays a seminal role in the pathogenesis of AD and can precede cognitive symptoms by years or decades. See, for example, Selkoe, 1991, Neuron 6:487. Release of A beta from neuronal cells grown in culture and the presence of A beta in cerebrospinal fluid (CSF) of both normal individuals and AD subjects has been demonstrated. See, for example, Seubert et al., 1992, Nature 359:325-327.

It has been proposed that A beta peptide accumulates as a result of APP processing by beta-secretase, thus inhibition of this enzyme's activity is desirable for the treatment of AD. In vivo processing of APP at the beta-secretase cleavage site is thought to be a rate-limiting step in A beta production, and is thus a therapeutic target for the treatment of AD. See for example, Sabbagh, M., et al., 1997, Alz. Dis. Rev. 3, 1-19.

BACE1 knockout mice fail to produce A beta, and present a normal phenotype. When crossed with transgenic mice that over express APP, the progeny show reduced amounts of A beta in brain extracts as compared with control animals (Luo et al., 2001 Nature Neuroscience 4:231-232). This evidence further supports the proposal that inhibition of beta-secretase activity and reduction of A beta in the brain provides a therapeutic method for the treatment of AD and other beta amyloid disorders. At present there are no effective treatments for halting, preventing, or reversing the progression of Alzheimer's disease. Therefore, there is an urgent need for pharmaceutical agents capable of slowing the progression of Alzheimer's disease and/or preventing it in the first place.

Compounds that are effective inhibitors of beta-secretase, that inhibit beta-secretase-mediated cleavage of APP, that are effective inhibitors of A beta production, and/or are effective to reduce amyloid beta deposits or plaques, are needed for the treatment and prevention of disease characterized by amyloid beta deposits or plaques, such as AD.

At present there are no effective treatments for halting, preventing, or reversing the progression of Alzheimer's disease. Therefore, there is an urgent need for pharmaceutical agents capable of slowing the progression of Alzheimer's disease and/or preventing it in the first place.

Compounds that are effective inhibitors of beta-secretase, that inhibit beta-secretase-mediated cleavage of APP, that are effective inhibitors of A beta production, and/or are effective to reduce amyloid beta deposits or plaques, are needed for the treatment and prevention of disease characterized by amyloid beta deposits or plaques, such as AD.

### SUMMARY OF INVENTION

The present invention relates to methods of treating a subject who has, or in preventing a subject from developing, a disease or condition selected from the group consisting of Alzheimer's disease, for helping prevent or delay the onset of Alzheimer's disease, for helping to slow the progression of Alzheimer's disease, for treating subjects with mild cognitive impairment (MCI) and preventing or delaying the onset of Alzheimer's disease in those who would progress from MCI to AD, for treating Down's syndrome, for treating humans who have Hereditary Cerebral Hemorrhage with Amyloidosis of the Dutch-Type, for treating cerebral amyloid angiopathy and preventing its potential consequences, i.e. single and recurrent lobar hemorrhages, for treating other degenerative dementias, including dementias of mixed vascular and degenerative origin, dementia associated with Parkinson's disease, frontotemporal dementias with parkinsonism (FTDP), dementia associated with progressive supranuclear palsy, dementia associated with cortical basal degeneration, or diffuse Lewy body type of Alzheimer's disease and who is in need of such treatment which comprises administration of a therapeutically effective amount of a compound described in U.S. Pat. No. 5,413,999, i.e., a compound of formula (I) 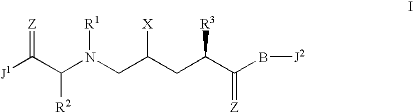
wherein
X is —OH or —NH₂;
Z is —O, —S, or —NH;
R is hydrogen or C₁₋₄ alkyl;
R¹ and R² are independently:
   1) hydrogen,
   2) —C₁₋₄ alkyl unsubstituted or substituted with one or more of
      a) halo,
      b) hydroxy,
      c) C₁₋₃ alkoxy,
      d) aryl unsubstituted or substituted with one or more of C₁₋₄ alkyl, halo, amino, hydroxy or aryl,
      e) —W-aryl or —W-benzyl, wherein W is —O—, —S—, or —NH—,
      f) a 5-7 membered cycloalkyl group unsubstituted or substituted with one or more of
         i) halo,
         ii) hydroxy,
         iii) C₁₋₃ alkoxy,
         iv) aryl,
      g) heterocycle unsubstituted or substituted with one or more of hydroxy, oxo, halo, C₁₋₄ alkoxy, C₁₋₄ alkyl optionally substituted with hydroxy; 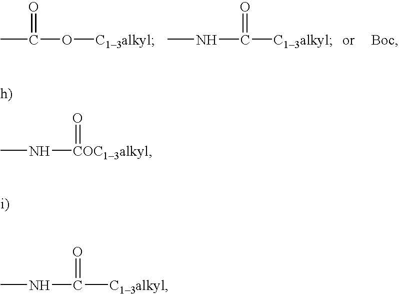
      j) —NH—SO₂C₁₋₃alkyl,
      k) —NR₂,
      l) —COOR, or
      m) —((CH₂)ₘO)ₙR wherein m is 2-5 and n is zero, 1, 2 or 3, or
   3) aryl, unsubstituted or substituted with one or more of
      a) halo,
      b) hydroxy,
      c) —NO₂ or —NR₂,
      d) C₁₋₄alkyl,
      e) C₁₋₃alkoxy, unsubstituted or substituted with one or more of —OH or C₁₋₃alkoxy,
      f) —COOR, 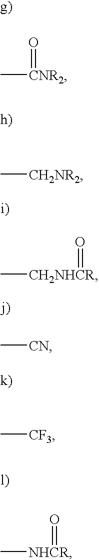
      m) aryl C₁₋₃alkoxy,
      n) aryl,
      o) —NRSO₂R,
      p) —OP(O)(ORₓ)₂, or
      q) —R⁵, as defined below; or
   4) heterocycle unsubstituted or substituted with one or more of hydroxy, oxo, halo, amino, C₁₋₄ alkoxy, C₁₋₄ alkyl optionally substituted with hydroxy; or Boc;
   5) carbocyclic unsubstituted or substituted with one or more of halo, amino, hydroxy or C₁₋₄ alkoxy;
R¹ and R² can be joined together to form with the nitrogen to which R¹ is attached a 3 to 10 membered monocyclic or bicyclic saturated ring system which consists of the nitrogen to which R¹ is attached and from 2 to 9 carbon atoms, and is unsubstituted or substituted with
   1) hydroxy,
   2) C₁₋₄ alkyl unsubstituted or substituted with one or more of
      a) halo,
      b) hydroxy,
      c) C₁₋₃ alkoxy,
      d) aryl,
      e) a 5-7 membered cycloalkyl group unsubstituted or substituted with one or more of
         i) halo,
         ii) hydroxy,
         iii) C₁₋₃ alkoxy, or
         iv) aryl,
      f) heterocycle, or
      g) —NR₂,
   3) C₁₋₃ alkoxy, 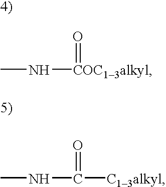
   6) —NH—SO₂C₁₋₃ alkyl,
   7) heterocycle,
   8) —W-aryl, or 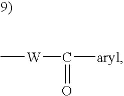
   wherein W is defined above; or
R¹ and R² can be joined together to form with the nitrogen to which R¹ is attached a 3 to 10 membered monocyclic or bicyclic saturated ring system which consists of the nitrogen to which R¹ is attached, from 1 to 8 carbon atoms and one or more unsubstituted or substituted heteroatom selected from 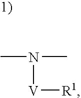
wherein V is absent or 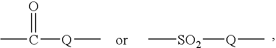
R¹ is defined as above for when R¹ is independent from and not joined to R², and wherein Q is absent or —O—, —NR—, or heterocycle optionally substituted with —C₁₋₄alkyl, 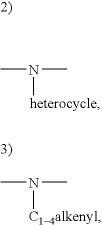
unsubstituted or substituted with aryl, 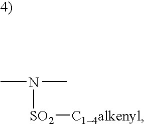
unsubstituted or substituted with aryl, 5) —S(O)p-, wherein p is zero, 1 or 2, or
   6) —O—; or
R¹ and R² can be joined together to form with the nitrogen to which R¹ is attached a 3 to 10 membered monocyclic or bicyclic saturated ring system, which consists of the nitrogen to which R¹ is attached and from 2 to 9 carbon atoms, in which the saturated ring system is fused to a phenyl ring and the phenyl ring is unsubstituted or substituted with one or more of
   1) halo,
   2) C₁₋₃ alkoxy,
   3) hydroxy,
   4) C₁₋₄ alkyl,
   5) —NHR¹, wherein R¹ is defined as above for when R¹ is independent from and not joined to R², or
   6) —NH-heterocycle;
R³ is
   1) —(CH₂)ᵣ—R⁴, wherein r is zero through 5,
   2) C₁₋₄alkenyl-R⁴,
   3) C₁₋₄ alkynyl-R⁴;
R¹ is
   1) hydrogen,
   2) C₁₋₄ alkyl,
   3) C₅-C₁₀ cycloalkyl, optionally substituted with hydroxy,
   4) C₆-C₁₀ aryl, unsubstituted or substituted with one or more of
      a) halo,
      b) hydroxy,
      c) —NO₂ or —NR²,
      d) C₁₋₄ alkyl,
      e) C₁₋₃ alkoxy, unsubstituted or substituted with one or more of —OH or C₁₋₃ alkoxy, 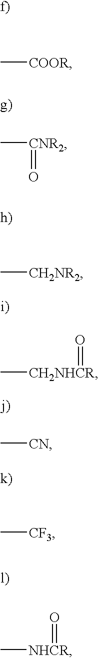
      m) aryl C₁₋₃ alkoxy, n) aryl,
      o) —NRSO₂R,
      p) —OP(O)(ORₓ)₂, or
      q) —R⁵, as defined below, or
   5) monocyclic or bicyclic heterocyle containing from 1 to 3 heteroatoms chosen from the group consisting of N, O, and S and which is unsubstituted or substituted with R⁵ and optionally with one or more of
      a) halo,
      b) C₁₋₄ alkyl, or
      c) C₁₋₃ alkoxy;
      Rₓ is H or aryl;
R⁵ is
   1) —W—(CH₂)ₘ—NR⁶R⁷ wherein W is as defined above, m is 2-5, and R⁶ and R⁷ are independently
      a) hydrogen,
      b) C₁₋₆ alkyl, unsubstituted or substituted with one or more of
         i) C₁₋₃ alkoxy,
         ii) —OH, or
         iii) —NR₂,
      c) the same or different and joined together to form a 5-7 member heterocycle, such as morpholino, containing up to two additional heteroatoms selected from 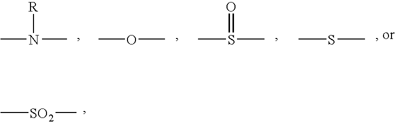
      the heterocycle optionally substituted with C₁₋₄ alkyl, or
      d) aromatic heterocycle unsubstituted or substituted with one or more of
         i) C₁₋₄ alkyl, or
         ii) —NR₂,
   2) —(CH₂)_{q}—NR⁶R⁷ wherein q is 1-5, and R⁶ and R⁷ are defined above, except that R⁶ or R⁷ are not H or unsubstituted C₁₋₆ alkyl, or
   3) benzofuryl, indolyl, azacycloalkyl, azabicyclo C₇₋₁₁ cycloalkyl, or benzopiperidinyl, unsubstituted or substituted with C₁₋₄ alkyl;
   B is absent, or 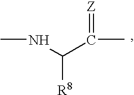
wherein R¹ is 1) —CH (CH₃)₂,
   2) —CH(CH₃)(CH₂CH₃), or
   3) -phenyl;
J¹ and J² are independently
   1) —YR⁹ wherein Y is —O— or —NH—, and R⁹ is
      a) hydrogen,
      b) C₁₋₆ alkyl, unsubstituted or substituted with one or more of
         i) —NR₂,
         ii) —OR,
         iii) —NHSO₂C₁₋₄ alkyl,
         iv) NHSO₂aryl, or —NHSO₂ (dialkylaminoaryl),
         v) —CH₂OR,
         vi) —C₁₋₄alkyl, 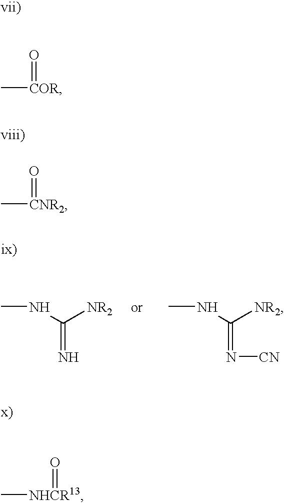
      wherein R¹³ is
         A) —H
         B) —C₁₋₄ alkyl,
         C) -aryl,
         D) -heterocycle, or
         E) —NH—, —O— or —(CH₂)ₙ— wherein n is zero, 1, 2 or 3, substituted with
         I) —C₁₋₄ alkyl, unsubstituted or substituted with one or more of aryl or heterocycle, or
         II) aryl, unsubstituted or substituted with heterocycle,
         xi) —NR₃^{⊕}A^{⊖} wherein A^{⊖} is a counterion,
         xii) —NR¹⁰R¹¹ wherein R¹⁰ and R¹¹ are the same or different and are C₁₋₅ alkyl joined together directly to form a 5-7 membered heterocycle containing up to one additional heteroatom selected from —O—, —S—, or —NR—,
         xiii) aryl,
         xiv) —CHO,
         xv) —OP(O)(ORₓ)₂, 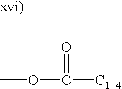
         alkyl substituted with one or more of amine or quaternary amine, or —O—((CH₂)ₘO)ₙ—R, or —OP(O)(ORₓ)₂, 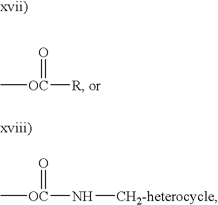
      or
         c) —((CH₂)ₘO)ₙCH₃ or —((CH₂)ₘO)ₙH, wherein m and n are defined above,
   2) —N(R⁹)ₓ,
   3) —NR¹⁰R¹¹ wherein R¹⁰ and R¹¹ are defined above, or 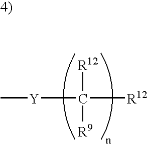
   wherein Y, R⁹ and n are defined above; and
R¹² is
   1) hydrogen,
   2) aryl, unsubstituted or substituted with one or more of
      a) R¹⁴, wherein R¹⁴ is 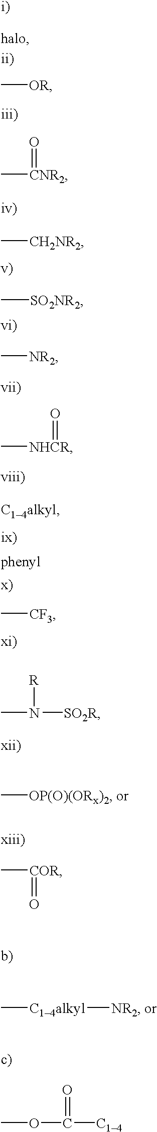
      alkyl substituted with one or more of amine or quaternary amine or —OP(O)(ORₓ)₂,
   3) heterocycle, such as isochroman, chroman, isothiochroman, thiochroman, benzimidazole, benzothiopyran, oxobenzothiopyran, benzopyran, benzothiopyranylsulfone, benzothiopyranylsulfoxide, the ring or rings being unsubstituted or substituted with one or more of
      a) R¹⁴, as defined above,
      b) —OC₁₋₄ alkenyl,
      c) phenyl-C₁₋₄ alkyl, 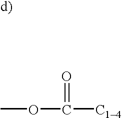
      alkyl substituted with one or more of amine or quaternary amine, or —OP(O)(ORₓ)₂, or 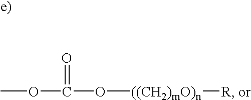
   4) A 5 to 7 membered carbocyclic or 7-10 membered bicyclic carbocyclic ring, such as cyclopentane, cyclohexane, indane, norbornane, naphthalene, thiopyran, isothiopyran, or benzopyran, the carbocyclic ring being unsubstituted or substituted with one or more of
      a) R¹⁴, as defined above,
      b) —CH₂OR,
      c) —(CH₂)ₙ—NR₂, C₅₋₁₆alkyl, pyridine, 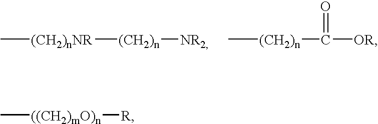
      quinuclidiniumyl substituted with R, piperazine-C₁₋₄ alkyl-benzyl substituted one or more with R, or morpholino-C₁₋₄ alkyl-benzyl, 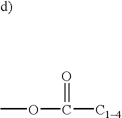
      alkyl substituted with one or more of amine or quaternary amine, —OP(ORₓ)₂ or 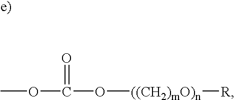
         or
      f) —C₁₋₄ alkyl-phenyl;
         or a pharmaceutically acceptable salt thereof.

The reader is directed to U.S. Pat. No. 5,413,999, which discloses compounds of formula (I) and their use as protease inhibitors for HIV, for methods of preparing the compounds employed in the methods of the invention. The disclosure of this document is incorporated herein by reference, in its entirety.

The present invention provides compounds, compositions, kits, and methods for inhibiting beta-secretase-mediated cleavage of amyloid precursor protein (APP). More particularly, the compounds, compositions, and methods of the invention are effective to inhibit the production of A beta peptide and to treat or prevent any human or veterinary disease or condition associated with a pathological form of A beta peptide.

The compounds, compositions, and methods of the invention are useful for treating humans who have Alzheimer's Disease (AD), for helping prevent or delay the onset of AD, for treating patients with mild cognitive impairment (MCI), and preventing or delaying the onset of AD in those patients who would otherwise be expected to progress from MCI to AD, for treating Down's syndrome, for treating Hereditary Cerebral Hemorrhage with Amyloidosis of the Dutch Type, for treating cerebral beta-amyloid angiopathy and preventing its potential consequences such as single and recurrent lobar hemorrhages, for treating other degenerative dementias, including dementias of mixed vascular and degenerative origin, for treating dementia associated with Parkinson's disease, dementia associated with progressive supranuclear palsy, dementia associated with cortical basal degeneration, and diffuse Lewy body type AD.

The compounds employed in the methods of the invention possess beta-secretase inhibitory activity. The inhibitory activities of the compounds employed in the methods of the invention are readily demonstrated, for example, using one or more of the assays described herein or known in the art.

### DETAILED DESCRIPTION OF THE INVENTION

U.S. Pat. No. 5,413,999 discloses various compounds of the formula I 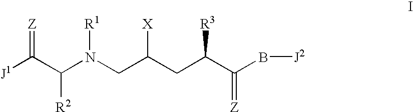
where R₁, R₂, R₃, B, J¹, J², X and Z are as defined above, and which are useful for the inhibition of the HIV protease enzyme.

U.S. Pat. No. 5,413,999 discloses how to make the above compounds and how to use them for the inhibition of the HIV protease enzyme. The essential material of U.S. Pat. No. 5,413,999 with regard to how to make these compounds is incorporated herein by reference.

In one aspect, the present invention relates to methods of treating a subject who has, or in preventing a subject from developing, a disease or condition selected from the group consisting of Alzheimer's disease, for helping prevent or delay the onset of Alzheimer's disease, for helping to slow the progression of Alzheimer's disease, for treating subjects with mild cognitive impairment (MCI) and preventing or delaying the onset of Alzheimer's disease in those who would progress from MCI to AD, for treating Down's syndrome, for treating humans who have Hereditary Cerebral Hemorrhage with Amyloidosis of the Dutch-Type, for treating cerebral amyloid angiopathy and preventing its potential consequences, i.e. single and recurrent lobar hemorrhages, for treating other degenerative dementias, including dementias of mixed vascular and degenerative origin, dementia associated with Parkinson's disease, frontotemporal dementias with parkinsonism (FTDP), dementia associated with progressive supranuclear palsy, dementia associated with cortical basal degeneration, or diffuse Lewy body type of Alzheimer's disease and who is in need of such treatment which comprises administration of a therapeutically effective amount of a compound of formula (I), or pharmaceutically acceptable salts thereof: 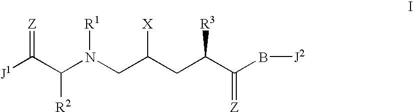
wherein
X is —OH or —NH₂;
Z is —O, —S, or —NH;
R is hydrogen or C₁₋₄ alkyl;
R¹ and R² are independently:
   1) hydrogen,
   2) —C₁₋₄ alkyl unsubstituted or substituted with one or more of
      a) halo,
      b) hydroxy,
      c) C₁₋₃ alkoxy,
      d) aryl unsubstituted or substituted with one or more of C₁₋₄ alkyl, halo, amino, hydroxy or aryl,
      e) —W-aryl or —W-benzyl, wherein W is —O—, —S—, or —NH—,
      f) a 5-7 membered cycloalkyl group unsubstituted or substituted with one or more of
         i) halo,
         ii) hydroxy,
         iii) C₁₋₃ alkoxy,
         iv) aryl,
      g) heterocycle unsubstituted or substituted with one or more of hydroxy, oxo, halo, C₁₋₄ alkoxy, C₁₋₄ alkyl optionally substituted with hydroxy; 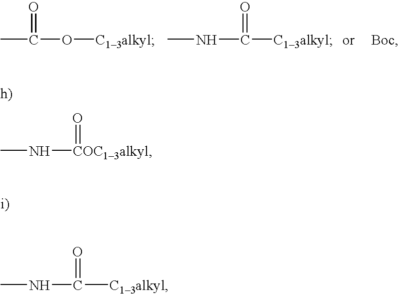
      j) —NH—SO₂C₁₋₃alkyl,
      k) —NR₂,
      l) —COOR, or
      m) —((CH₂)ₘO)ₙR wherein m is 2-5 and n is zero, 1, 2 or 3, or
   3) aryl, unsubstituted or substituted with one or more of
      a) halo,
      b) hydroxy,
      c) —NO₂ or —NR₂,
      d) C₁₋₄alkyl,
      e) C₁₋₃alkoxy, unsubstituted or substituted with one or more of —OH or C₁₋₃alkoxy, 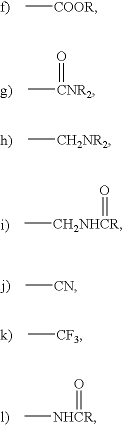
      m) aryl C₁₋₃alkoxy,
      n) aryl,
      o) —NRSO₂R,
      p) —OP(O)(ORₓ)₂, or
      q) —R⁵, as defined below; or
   4) heterocycle unsubstituted or substituted with one or more of hydroxy, oxo, halo, amino, C₁₋₄ alkoxy, C₁₋₄ alkyl optionally substituted with hydroxy; or Boc;
   5) carbocyclic unsubstituted or substituted with one or more of halo, amino, hydroxy or C₁₋₄ alkoxy;
R¹ and R² can be joined together to form with the nitrogen to which R¹ is attached a 3 to 10 membered monocyclic or bicyclic saturated ring system which consists of the nitrogen to which R¹ is attached and from 2 to 9 carbon atoms, and is unsubstituted or substituted with
   1) hydroxy,
   2) C₁₋₄ alkyl unsubstituted or substituted with one or more of
      a) halo,
      b) hydroxy,
      c) C₁₋₃ alkoxy,
      d) aryl,
      e) a 5-7 membered cycloalkyl group unsubstituted or substituted with one or more of
         i) halo,
         ii) hydroxy,
         iii) C₁₋₃ alkoxy, or
         iv) aryl,
      f) heterocycle, or
      g) —NR₂,
   3) C₁₋₃ alkoxy, 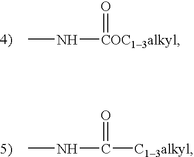
   6) —NH—SO₂C₁₋₃ alkyl,
   7) heterocycle,
   8) —W-aryl, or 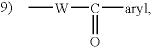
   wherein W is defined above; or
R¹ and R² can be joined together to form with the nitrogen to which R¹ is attached a 3 to 10 membered monocyclic or bicyclic saturated ring system which consists of the nitrogen to which R¹ is attached, from 1 to 8 carbon atoms and one or more unsubstituted or substituted heteroatom selected from 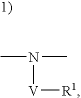
wherein V is absent or 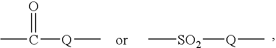
R¹ is defined as above for when R¹ is independent from and not joined to R², and wherein Q is absent or —O—, —NR—, or heterocycle optionally substituted with —C₁₋₄alkyl, 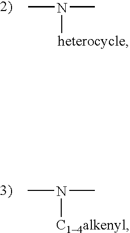
unsubstituted or substituted with aryl, 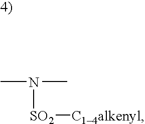
unsubstituted or substituted with aryl, 5) —S(O)p-, wherein p is zero, 1 or 2, or
   6) —O—; or
R¹ and R² can be joined together to form with the nitrogen to which R¹ is attached a 3 to 10 membered monocyclic or bicyclic saturated ring system, which consists of the nitrogen to which R¹ is attached and from 2 to 9 carbon atoms, in which the saturated ring system is fused to a phenyl ring and the phenyl ring is unsubstituted or substituted with one or more of
   1) halo,
   2) C₁₋₃ alkoxy,
   3) hydroxy,
   4) C₁₋₄ alkyl,
   5) —NHR¹, wherein R¹ is defined as above for when R¹ is independent from and not joined to R², or
   6) —NH-heterocycle;
R³ is
   1) —(CH₂)ᵣ—R⁴, wherein r is zero through 5,
   2) C₁₋₄alkenyl-R⁴,
   3) C₁₋₄ alkynyl-R⁴;
   R¹ is
   1) hydrogen,
   2) C₁₋₄ alkyl,
   3) C₅-C₁₀ cycloalkyl, optionally substituted with hydroxy,
   4) C₆-C₁₀ aryl, unsubstituted or substituted with one or more of
      a) halo,
      b) hydroxy,
      c) —NO₂ or —NR₂,
      d) C₁₋₄ alkyl,
      e) C₁₋₃ alkoxy, unsubstituted or substituted with one or more of —OH or C₁₋₃ alkoxy, 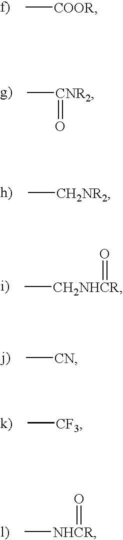
      m) aryl C₁₋₃ alkoxy, n) aryl,
      o) —NRSO₂R,
      p) —OP(O)(ORₓ)₂, or
      q) —R⁵, as defined below, or
   5) monocyclic or bicyclic heterocyle containing from 1 to 3 heteroatoms chosen from the group consisting of N, O, and S and which is unsubstituted or substituted with R⁵ and optionally with one or more of
      a) halo,
      b) C₁₋₄ alkyl, or
      c) C₁₋₃ alkoxy;
      Rₓ is H or aryl;
R⁵ is
   1) —W—(CH₂)ₘ—NR⁶R⁷ wherein W is as defined above, m is 2-5, and R⁶ and R⁷ are independently
      a) hydrogen,
      b) C₁₋₆ alkyl, unsubstituted or substituted with one or more of
         i) C₁₋₃ alkoxy,
         ii) —OH, or
         iii) —NR₂,
      c) the same or different and joined together to form a 5-7 member heterocycle, such as morpholino, containing up to two additional heteroatoms selected from 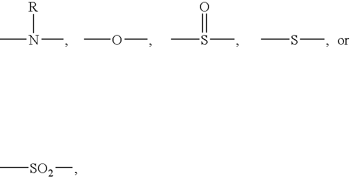
      the heterocycle optionally substituted with C₁₋₄ alkyl, or
      d) aromatic heterocycle unsubstituted or substituted with one or more of
         i) C₁₋₄ alkyl, or
         ii) —NR₂,
   2) —(CH₂)_{q}—NR⁶R⁷ wherein q is 1-5, and R⁶ and R⁷ are defined above, except that R⁶ or R⁷ are not H or unsubstituted C₁₋₆ alkyl, or
   3) benzofuryl, indolyl, azacycloalkyl, azabicyclo C₇₋₁₁ cycloalkyl, or benzopiperidinyl, unsubstituted or substituted with C₁₋₄ alkyl;
   B is absent, or 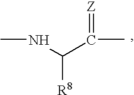
wherein R¹ is 1) —CH(CH₃)₂,
   2) —CH(CH₃)(CH₂CH₃), or
   3) -phenyl;
J¹ and J² are independently
   1) —YR⁹ wherein Y is —O— or —NH—, and R⁹ is
      a) hydrogen,
      b) C₁₋₆ alkyl, unsubstituted or substituted with one or more of
         i) —NR₂,
         ii) —OR,
         iii) —NHSO₂C₁₋₄ alkyl,
         iv) NHSO₂aryl, or —NHSO₂(dialkylaminoaryl),
         v) —CH₂OR,
         vi) —C₁₋₄ alkyl, 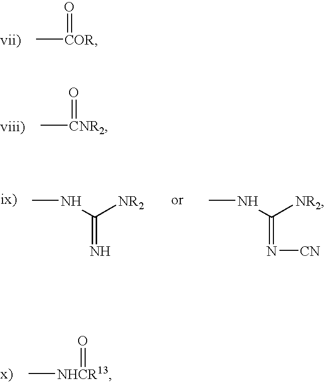
      wherein R¹³ is
         A) —H
         B) —C₁₋₄ alkyl,
         C) -aryl,
         D) -heterocycle, or
         E) —NH—, —O— or —(CH₂)ₙ— wherein n is zero, 1, 2 or 3, substituted with
         I) —C₁₋₄ alkyl, unsubstituted or substituted with one or more of aryl or heterocycle, or
         II) aryl, unsubstituted or substituted with heterocycle,
         xi) —NR₃^{⊕}A^{⊖} wherein A^{⊕} is a counterion,
         xii) —NR¹⁰R¹¹ wherein R¹⁰ and R¹¹ are the same or different and are C₁₋₅ alkyl joined together directly to form a 5-7 membered heterocycle containing up to one additional heteroatom selected from —O—, —S—, or —NR—,
         xiii) aryl,
         xiv) —CHO,
         xv) —OP(O)(ORₓ)₂, 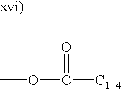
         alkyl substituted with one or more of amine or quaternary amine, or —O—((CH₂)ₘO)ₙ—R, or —OP(O)(ORₓ)₂, 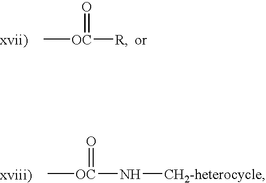
   or
      c) —((CH₂)ₘO)ₙCH₃ or —((CH₂)ₘO)ₙH, wherein m and n are defined above,
   2) —N(R⁹)ₓ,
   3) —NR¹⁰R¹¹ wherein R¹⁰ and R¹¹ are defined above, or 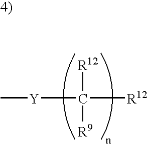
   wherein Y, R⁹ and n are defined above; and
R¹² is
   1) hydrogen,
   2) aryl, unsubstituted or substituted with one or more of
      a) R¹⁴, wherein R¹⁴ is 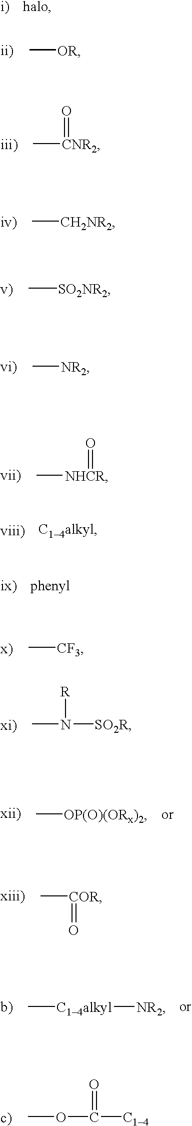
      alkyl substituted with one or more of amine or quaternary amine or —OP(O)(ORₓ)₂,
   3) heterocycle, such as isochroman, chroman, isothiochroman, thiochroman, benzimidazole, benzothiopyran, oxobenzothiopyran, benzopyran, benzothiopyranylsulfone, benzothiopyranylsulfoxide, the ring or rings being unsubstituted or substituted with one or more of
      a) R¹⁴, as defined above,
      b) —OC₁₋₄ alkenyl,
      c) phenyl-C₁₋₄ alkyl, 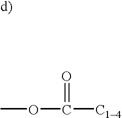
      alkyl substituted with one or more of amine or quaternary amine, or —OP(O)(ORₓ)₂, or 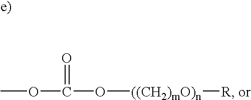
   4) A 5 to 7 membered carbocyclic or 7-10 membered bicyclic carbocyclic ring, such as cyclopentane, cyclohexane, indane, norbornane, naphthalene, thiopyran, isothiopyran, or benzopyran, the carbocyclic ring being unsubstituted or substituted with one or more of
      a) R^{14,} as defined above,
      b) —CH₂OR,
      c) —(CH₂)ₙ—NR², C₅-₁₆alkyl, pyridine, 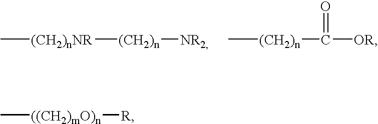
      quinuclidiniumyl substituted with R, piperazine-C₁₋₄ alkyl-benzyl substituted one or more with R, or morpholino-C₁₋₄ alkyl-benzyl, 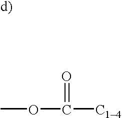
      alkyl substituted with one or more of amine or quaternary amine, —OP(ORₓ)₂ or 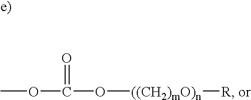
         or
      f) —C₁₋₄ alkyl-phenyl.

In one aspect, this method of treatment can be used where the disease is Alzheimer's disease.

In another aspect, this method of treatment can help prevent or delay the onset of Alzheimer's disease.

In another aspect, this method of treatment can help slow the progression of Alzheimer's disease.

In another aspect, this method of treatment can be used where the disease is mild cognitive impairment.

In another aspect, this method of treatment can be used where the disease is Down's syndrome.

In another aspect, this method of treatment can be used where the disease is Hereditary Cerebral Hemorrhage with Amyloidosis of the Dutch-Type.

In another aspect, this method of treatment can be used where the disease is cerebral amyloid angiopathy.

In another aspect, this method of treatment can be used where the disease is degenerative dementias.

In another aspect, this method of treatment can be used where the disease is diffuse Lewy body type of Alzheimer's disease.

In another aspect, this method of treatment can treat an existing disease, such as those listed above.

In another aspect, this method of treatment can prevent a disease, such as those listed above, from developing or progressing.

The methods of the invention employ therapeutically effective amounts: for oral administration from about 0.1 mg/day to about 1,000 mg/day; for parenteral, sublingual, intranasal, intrathecal administration from about 0.5 to about 100 mg/day; for depo administration and implants from about 0.5 mg/day to about 50 mg/day; for topical administration from about 0.5 mg/day to about 200 mg/day; for rectal administration from about 0.5 mg to about 500 mg.

In a preferred aspect, the therapeutically effective amounts for oral administration is from about 1 mg/day to about 100 mg/day; and for parenteral administration from about 5 to about 50 mg daily.

In a more preferred aspect, the therapeutically effective amounts for oral administration is from about 5 mg/day to about 50 mg/day.

The present invention also includes the use of a compound of formula (I), or a pharmaceutically acceptable salt thereof for the manufacture of a medicament for use in treating a subject who has, or in preventing a subject from developing, a disease or condition selected from the group consisting of Alzheimer's disease, for helping prevent or delay the onset of Alzheimer's disease, for treating subjects with mild cognitive impairment (MCI) and preventing or delaying the onset of Alzheimer's disease in those who would progress from MCI to AD, for treating Down's syndrome, for treating humans who have Hereditary Cerebral Hemorrhage with Amyloidosis of the Dutch-Type, for treating cerebral amyloid angiopathy and preventing its potential consequences, i.e. single and recurrent lobar hemorrhages, for treating other degenerative dementias, including dementias of mixed vascular and degenerative origin, dementia associated with Parkinson's disease, frontotemporal dementias with parkinsonism (FTDP), dementia associated with progressive supranuclear palsy, dementia associated with cortical basal degeneration, diffuse Lewy body type of Alzheimer's disease and who is in need of such treatment.

In one aspect, this use of a compound of formula (I) can be employed where the disease is Alzheimer's disease.

In another aspect, this use of a compound of formula (I) can help prevent or delay the onset of Alzheimer's disease.

In another aspect, this use of a compound of formula (I) can help slow the progression of Alzheimer's disease.

In another aspect, this use of a compound of formula (I) can be employed where the disease is mild cognitive impairment.

In another aspect, this use of a compound of formula (I) can be employed where the disease is Down's syndrome.

In another aspect, this use of a compound of formula (I) can be employed where the disease is Hereditary Cerebral Hemorrhage with Amyloidosis of the Dutch-Type.

In another aspect, this use of a compound of formula (I) can be employed where the disease is cerebral amyloid angiopathy.

In another aspect, this use of a compound of formula (I) can be employed where the disease is degenerative dementias.

In another aspect, this use of a compound of formula (I) can be employed where the disease is diffuse Lewy body type of Alzheimer's disease.

In a preferred aspect, this use of a compound of formula (I) is a pharmaceutically acceptable salt of an acid selected from the group consisting of acids hydrochloric, hydrobromic, hydroiodic, nitric, sulfuric, phosphoric, citric, methanesulfonic, CH₃—(CH₂)ₙ—COOH where n is 0 thru 4, HOOC—(CH₂)ₙ—COOH where n is as defined above, HOOC—CH═CH—COOH, and phenyl-COOH.

In another preferred aspect of the invention, the subject or patient is preferably a human subject or patient.

The present invention also includes methods for inhibiting beta-secretase activity, for inhibiting cleavage of amyloid precursor protein (APP), in a reaction mixture, at a site between Met596 and Asp597, numbered for the APP-695 amino acid isotype, or at a corresponding site of an isotype or mutant thereof; for inhibiting production of amyloid beta peptide (A beta) in a cell; for inhibiting the production of beta-amyloid plaque in an animal; and for treating or preventing a disease characterized by beta-amyloid deposits in the brain. These methods each include administration of a therapeutically effective amount of a compound of formula (I), or a pharmaceutically acceptable salt thereof.

The present invention also includes a method for inhibiting beta-secretase activity, including exposing said beta-secretase to an effective inhibitory amount of a compound of formula (I), or a pharmaceutically acceptable salt thereof.

In one aspect, this method includes exposing said beta-secretase to said compound in vitro.

In another aspect, this method includes exposing said beta-secretase to said compound in a cell.

In another aspect, this method includes exposing said beta-secretase to said compound in a cell in an animal.

In another aspect, this method includes exposing said beta-secretase to said compound in a human.

The present invention also includes a method for inhibiting cleavage of amyloid precursor protein (APP), in a reaction mixture, at a site between Met596 and Asp597, numbered for the APP-695 amino acid isotype; or at a corresponding site of an isotype or mutant thereof, including exposing said reaction mixture to an effective inhibitory amount of a compound of formula (I), or a pharmaceutically acceptable salt thereof.

In one aspect, this method employs a cleavage site: between Met652 and Asp653, numbered for the APP-751 isotype; between Met 671 and Asp 672, numbered for the APP-770 isotype; between Leu596 and Asp597 of the APP-695 Swedish Mutation; between Leu652 and Asp653 of the APP-751 Swedish Mutation; or between Leu671 and Asp672 of the APP-770 Swedish Mutation.

In another aspect, this method exposes said reaction mixture in vitro.

In another aspect, this method exposes said reaction mixture in a cell.

In another aspect, this method exposes said reaction mixture in an animal cell.

In another aspect, this method exposes said reaction mixture in a human cell.

The present invention also includes a method for inhibiting production of amyloid beta peptide (A beta) in a cell, including administering to said cell an effective inhibitory amount of a compound of formula (I), or a pharmaceutically acceptable salt thereof.

In an embodiment, this method includes administering to an animal.

In an embodiment, this method includes administering to a human.

The present invention also includes a method for inhibiting the production of beta-amyloid plaque in an animal, including administering to said animal an effective inhibitory amount of a compound of formula (I), or a pharmaceutically acceptable salt thereof.

In one embodiment of this aspect, this method includes administering to a human.

The present invention also includes a method for treating or preventing a disease characterized by beta-amyloid deposits in the brain including administering to a subject an effective therapeutic amount of a compound of formula (I), or a pharmaceutically acceptable salt thereof.

In one aspect, this method employs a compound at a therapeutic amount in the range of from about 0.1 to about 1000 mg/day.

In another aspect, this method employs a compound at a therapeutic amount in the range of from about 15 to about 1500 mg/day.

In another aspect, this method employs a compound at a therapeutic amount in the range of from about 1 to about 100 mg/day.

In another aspect, this method employs a compound at a therapeutic amount in the range of from about 5 to about 50 mg/day.

In another aspect, this method can be used where said disease is Alzheimer's disease.

In another aspect, this method can be used where said disease is Mild Cognitive Impairment, Down's Syndrome, or Hereditary Cerebral Hemorrhage with Amyloidosis of the Dutch Type.

The present invention also includes a composition including beta-secretase complexed with a compound of formula (I), or a pharmaceutically acceptable salt thereof.

The present invention also includes a method for producing a beta-secretase complex including exposing beta-secretase to a compound of formula (I), or a pharmaceutically acceptable salt thereof, in a reaction mixture under conditions suitable for the production of said complex.

In an embodiment, this method employs exposing in vitro.

In an embodiment, this method employs a reaction mixture that is a cell.

The present invention also includes a component kit including component parts capable of being assembled, in which at least one component part includes a compound of formula (I) enclosed in a container.

In an embodiment, this component kit includes lyophilized compound, and at least one further component part includes a diluent.

The present invention also includes a container kit including a plurality of containers, each container including one or more unit dose of a compound of formula (I), or a pharmaceutically acceptable salt thereof.

In an embodiment, this container kit includes each container adapted for oral delivery and includes a tablet, gel, or capsule.

In an embodiment, this container kit includes each container adapted for parenteral delivery and includes a depot product, syringe, ampoule, or vial.

In an embodiment, this container kit includes each container adapted for topical delivery and includes a patch, medipad, ointment, or cream.

The present invention also includes an agent kit including a compound of formula (I), or a pharmaceutically acceptable salt thereof; and one or more therapeutic agents selected from the group consisting of an antioxidant, an anti-inflammatory, a gamma secretase inhibitor, a neurotrophic agent, an acetyl cholinesterase inhibitor, a statin, an A beta peptide, and an anti-A beta antibody.

The present invention provides compounds, compositions, kits, and methods for inhibiting beta-secretase-mediated cleavage of amyloid precursor protein (APP). More particularly, the compounds, compositions, and methods of the invention are effective to inhibit the production of A beta peptide and to treat or prevent any human or veterinary disease or condition associated with a pathological form of A beta peptide.

The compounds, compositions, and methods of the invention are useful for treating humans who have Alzheimer's Disease (AD), for helping prevent or delay the onset of AD, for treating subjects with mild cognitive impairment (MCI), and preventing or delaying the onset of AD in those subjects who would otherwise be expected to progress from MCI to AD, for treating Down's syndrome, for treating Hereditary Cerebral Hemorrhage with Amyloidosis of the Dutch Type, for treating cerebral beta-amyloid angiopathy and preventing its potential consequences such as single and recurrent lobar hemorrhages, for treating other degenerative dementias, including dementias of mixed vascular and degenerative origin, for treating dementia associated with Parkinson's disease, frontotemporal dementias with parkinsonism (FTDP), dementia associated with progressive supranuclear palsy, dementia associated with cortical basal degeneration, and diffuse Lewy body type AD.

The compounds of the invention possess beta-secretase inhibitory activity. The inhibitory activities of the compounds of the invention are readily demonstrated, for example, using one or more of the assays described herein or known in the art.

The present invention provides kits, and methods for inhibiting beta-secretase enzyme activity and A beta peptide production. Inhibition of beta-secretase enzyme activity halts or reduces the production of A beta from APP and reduces or eliminates the formation of beta-amyloid deposits in the brain.

In one aspect of the methods of the invention, compounds of formula (I) comprise a structure wherein R¹ and R² are joined together to form with the nitrogen to which R¹ is attached a 3 to 10 membered monocyclic or bicyclic saturated ring system which consists of the nitrogen to which R¹ is attached and from 2 to 9 carbon atoms, and is unsubstituted or substituted with
1) hydroxy,
2) C₁₋₄ alkyl unsubstituted or substituted with one or more of
   a) hydroxy,
   b) C₁₋₃ alkoxy,
   c) aryl,
   d) a 5-7 membered cycloalkyl group unsubstituted or substituted with one or more of
      i) halo,
      ii) hydroxy,
      iii) C₁₋₃ alkoxy, or
      iv) aryl,
      e) heterocycle, or
      f) —NR₂,
3) C₁₋₆ alkoxy, 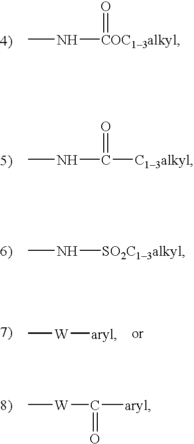
wherein W is -0-, —S—, or —NH—; or
R¹ and R² are joined together to form with the nitrogen to which R¹ is attached a 3 to 10 membered monocyclic or bicyclic saturated ring system which consists of the nitrogen to which R¹ is attached, from 1 to 8 carbon atoms and one or more unsubstituted or substituted heteroatom selected from 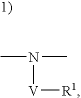
wherein V is absent or 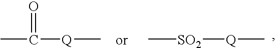
R¹ is defined as above for when R¹ is independent from and not joined to R², and wherein Q is absent or —O—, —NR—, or heterocycle optionally substituted with —C₁₋₄ alkyl, 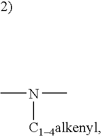
unsubstituted or substituted with aryl,
3) —S(O)p-, wherein p is zero, 1 or 2, or
4) —O—; or
R¹ and R² are joined together to form with the nitrogen to which R¹ is attached a 3 to 10 membered monocyclic or bicyclic saturated ring system, which consists of the nitrogen to which R¹ is attached and from 2 to 9 carbon atoms, in which the saturated ring system is fused to a phenyl ring and the phenyl ring is unsubstituted or substituted with one or more of
1) C₁₋₃ alkoxy,
2) hydroxy,
3) C₁₋₄ alkyl, or
4) —NHR¹, wherein R¹ is defined as above for when R¹ is independent from and not joined to R².

In another aspect, the methods comprise compounds for formula (I) wherein:
R¹ and R² are joined together to form with the nitrogen to which R¹ is attached a 3 to 10 membered monocyclic or bicyclic saturated ring system which consists of the nitrogen to which R¹ is attached and from 2 to 9 carbon atoms, and is unsubstituted or substituted with
1) hydroxy,
2) C₁₋₄ alkyl unsubstituted or substituted with one or more of
   a) hydroxy,
   b) C₁₋₃ alkoxy,
   c) aryl,
   d) a 5-7 membered cycloalkyl group unsubstituted or substituted with one or more of
      i) halo,
      ii) hydroxy,
      iii) C₁₋₃ alkoxy, or
      iv) aryl,
      e) heterocycle, or
      f) —NR₂,
3) C₁₋₃ alkoxy, 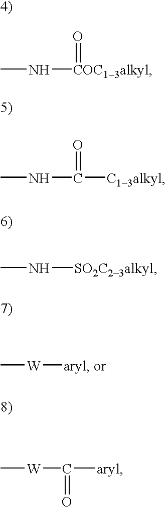
   wherein W is —O—, —S—, or —NH—; or
R¹ and R² are joined together to form with the nitrogen to which R¹ is attached a 3 to 10 membered monocyclic or bicyclic saturated ring system which consists of the nitrogen to which R¹ is attached, from 1 to 8 carbon atoms and one or more unsubstituted or substituted heteroatom selected from 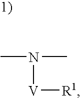
wherein V is absent or 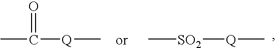
R¹ is defined as above for when R¹ is independent from and not joined to R², and wherein Q is absent or —O—, —NR—, or heterocycle optionally substituted with —C₁₋₄ alkyl,
2) —S(O)p-, wherein p is zero, 1 or 2, or
3) —O—;
R³ is benzyl, unsubstituted or substituted with one or more of
a) hydroxy,
b) —NO₂, or —NR₂,
c) C₁₋₄ alkyl,
d) C₁₋₃ alkoxy, unsubstituted or substituted with one or more of —OH or C₁₋₃ alkoxy, 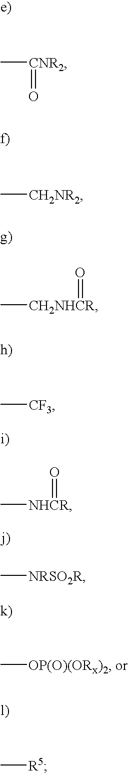
   and B is absent.

In another aspect, the methods comprise compounds for formula (I) wherein:
X is —OH;
Z is —O;
R¹ and R² are joined together to form with the nitrogen to which R¹ is attached a 3 to 10 membered monocyclic or bicyclic saturated ring system which consists of the nitrogen to which R¹ is attached and from 2 to 9 carbon atoms, and is unsubstituted or substituted with 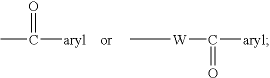
or;
R¹ and R² are joined together to form with the nitrogen to which R¹ is attached a 3 to 10 membered monocyclic or bicyclic saturated ring system which consists of the nitrogen to which R¹ is attached, from 1 to 8 carbon atoms and one of 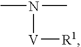
   wherein V is absent or 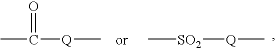
R¹ is defined as above for when R¹ is independent from and not joined to R², and wherein Q is absent or —O—, —NR— or heterocycle optionally substituted with —C₁₋₄ alkyl;
R³ is benzyl, unsubstituted or substituted with one or more of (1) hydroxy, (2) C₁₋₃ alkoxy substituted with one or more of —OH or (3) 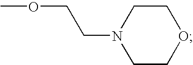
J¹ is —NH—C₁₋₄ alkyl; and
J² is

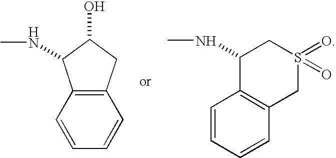

In another aspect, the methods comprise compounds of formula (I) the compounds are selected from the group consisting of compounds A through H and J, shown below.

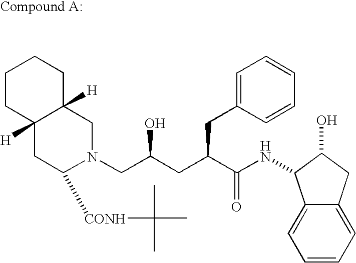

N-(2(R)-hydroxy-1(S)-indanyl)-2(R)-phenylmethyl-4(S)-hydroxy-5-(2-(3-(S)-N′-(t-butylcarboxamido)-(4aS,8aS)-decahydroisoquinoline)yl)-pentaneamide,

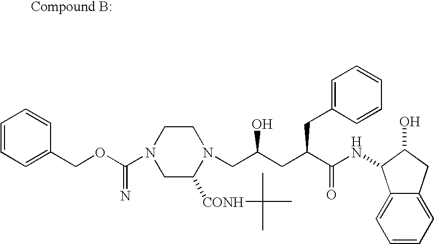

N-(2(R)-hydroxy-1(S)-indanyl)-2(R)-phenylmethyl(4(S)-hydroxy-5-(1-(4-carbobenzyloxy-2(S)-N′-(t-butylcarboxamido)-piperazinyl))-pentaneamide,

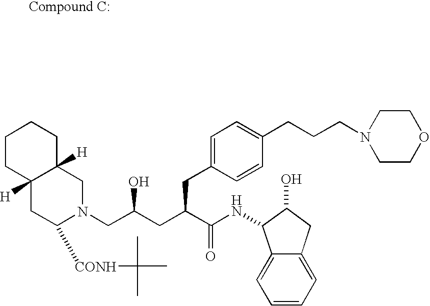

N-(2(R)-hydroxy-1(S)-indanyl)-2(R)-((4-(2-(4-morpholinyl)ethoxy)phenyl)methyl)-4(S)-hydroxy-5-(2-(3(S)-N′-(t-butylcarboxamido)-(4aS,8aS)-decahydroisoquinoline)yl)-pentaneamide,

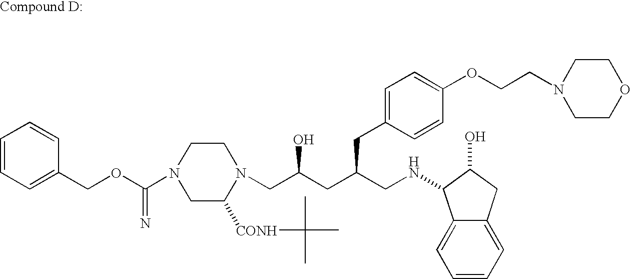

N-(2(R)-hydroxy-1(S)-indanyl)-2(R)-((4-(2-(4-morpholinyl)ethoxy)phenyl)methyl-4(S)-hydroxy-5-(1-(4-carbobenzyloxy-2(S)-N′-(t-butylcarboxamido)-piperazinyl))-pentaneamide,

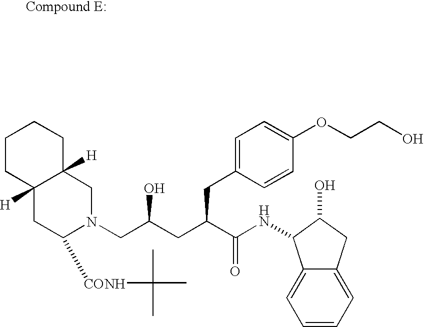

N-(2(R)-hydroxy-1(S)-indanyl)-2(R)-((4-((2-hydroxy)-ethoxy)phenyl)methyl)-4(S)-hydroxy-5-(2-(3(S)-N′-(t-butylcarboxamido)-(4aS,8aS)-decahydroisoquinoline)-yl)-pentaneamide,

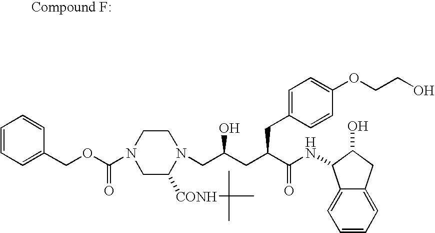

N-(2(R)-hydroxy-1(S)-indanyl)-2(R)-((4-((2-hydroxy)-ethoxy)phenyl)methyl)-4(S)-hydroxy-5-(1-(4-carbobenzyloxy-2(S)-N′-(t-butylcarboxamido)-piperazinyl))-pentaneamide,

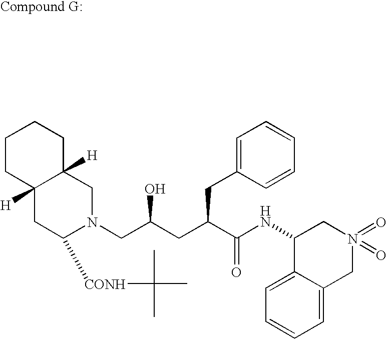

N-(4(S)-3,4-dihydro-1H-2,2-dioxobenzothiopyranyl)-2(R)-phenylmethyl-4(S)-hydroxy-5-(2-(3(S)-N′-(t-butylcarboxamido)-(4aS,8aS)-decahydroisoquinoline)yl)-pentaneamide,

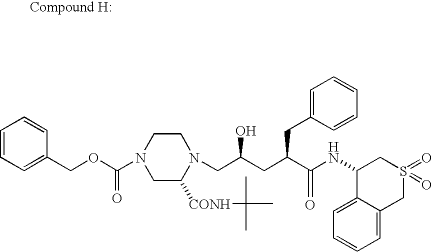

N-(4(S)-3,4-dihydro-1H-2,2-dioxobenzothiopyranyl)-(2(R)-phenylmethyl-4(S)-hydroxy-5-(1-(4-carbobenzyloxy-2(S)-N′-(t-butylcarboxamido)-piperazinyl))-pentaneamide,

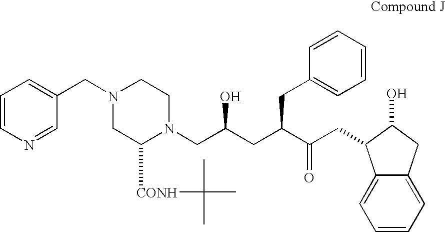

N-(2(R)-hydroxy-1(S)-indanyl)-2(R)-phenylmethyl-4(S)-hydroxy-5-(1-(4-(3-pyridylmethyl)-2(S)-N′(t-butylcarboxamido)-piperazinyl))-pentaneamide, alternatively named

[1S-[1α[αS*,γR*,δ(R*)],2α]]-N-(2,3-dihydro-2-hydroxy-1H-inden-1-yl)-2-[[(1,1-dimethylethyl)amino]carbonyl]-γ-hydroxy-α-(phenylmethyl)-4-(3-pyridinylmethyl)-1-piperazinepentaneamide, or

N-(1(S)-2,3-dihydro-2(R)-hydroxy-1H-indenyl)-4)S)-hydroxy-2-(R)-phenyl-methyl-5-[4-(3-pyridylmethyl)-2(S)-t-butylcarbamoyl)piperazinyl]pentaneamide.

Compounds employed in the novel methods of the present invention also include but are not limited to the following compounds:
N-(2(R)-hydroxy-1(S)-indanyl)-2(R)-phenylmethyl-4-(S)-hydroxy-5-(1-(N′-(t-butyl)-4(S)-phenoxyprolineamid)yl)-pentaneamide,
N-(2(R)-hydroxy-1(S)-indanyl)-2(R)-phenylmethyl-4-(S)-hydroxy-5-(1-(N′-t-butyl-4(S)-2-naphthyloxy-prolineamid)yl)-pentaneamide,
N-(2(R)-hydroxy-1(S)-indanyl)-2(R)-phenylmethyl-4-(S)-hydroxy-5-(1-(N′-t-butyl-4(S)-1-naphthyloxy-prolineamid)yl)-pentaneamide,
N-(2(R)-hydroxy-1(S)-indanyl)-2(R)-phenylmethyl-4-(S)-amino-5-(2-(3(S)-N′-(t-butylcarboxamido)-(4aS,8aS)-decahydroisoquinoline)yl)-pentaneamide,
N-(2(R)-hydroxy-1(S)-indanyl)-2(R)-phenylmethyl-4-(S)-hydroxy-5-(1-(4-(3-phenylpropionyl)-2(S)-N′-(t-butylcarboxamido)-piperazinyl))-pentaneamide,
N-(2(R)-hydroxy-1(S)-indanyl)-2(R)-phenylmethyl-4-(S)-hydroxy-5-(1-(4-benzoyl-2(S)-N′-(t-butylcarboxamido)-piperazinyl))-pentaneamide,
N-(2(R)-hydroxy-1(S)-indanyl)-2(R)-phenylmethyl-4-(S)-hydroxy-5-(1-(4-(3-phenylpropyl)-2(S)-N′-(t-butylcarboxamido)-piperazinyl))-pentaneamide,
N-(2(R)-hydroxy-1(S)-indanyl)-2(R)-phenylmethyl-4-(S)-amino-5-(1-(4-carbobenzyloxy-2(S)-N′-(t-butylcarboxamido)-piperazinyl))-pentaneamide,
N-(2(R)-hydroxy-1(S)-indanyl)-2(R)-((4-(2-(4-morpholinyl)ethoxy)phenyl)methyl)-4(S)-hydroxy-5-(1-(N′-(t-butyl)-4(S)-phenoxyprolineamid)yl)-pentaneamide,
N-(2(R)-hydroxy-1(S)-indanyl)-2(R)-((4-(2-(4-morpholinyl)ethoxy)phenyl)methyl)-4(S)-hydroxy-5-(1-(N′-t-butyl-4(S)-2-naphthyloxy-prolineamid)yl)-pentaneamide,
N-(2(R)-hydroxy-1(S)-indanyl)-2(R)-((4-(2-(4-morpholinyl)ethoxy)phenyl)methyl)-4(S)-hydroxy-5-(1-(N′-t-butyl-4(S)-1-naphthyloxy-prolineamid)yl)-pentaneamide,
N-(2(R)-hydroxy-1(S)-indanyl)-2(R)-((4-(2-(4-morpholinyl)ethoxy)phenyl)methyl)-4(S)-amino-5-(2-(3(S)-N′-(t-butylcarboxamido)-(4aS,8aS)-decahydroisoquinoline)yl)-pentaneamide,
N-(2(R)-hydroxy-1(S)-indanyl)-2(R)-((4-(2-(4-morpholinyl)ethoxy)phenyl)methyl)-4(S)-hydroxy-5-(1-(4-(3-phenylpropionyl)-2(S)-N′-(t-butylcarboxamido)-piperazinyl))-pentaneamide,
N-(2(R)-hydroxy-1(S)-indanyl)-2(R)-((4-(2-(4-morpholinyl)ethoxy)phenyl)methyl)-4(S)-hydroxy-5-(1-(4-benzoyl-2(S)-N′-(t-butylcarboxamido)-piperazinyl))-pentaneamide,
N-(2(R)-hydroxy-1(S)-indanyl)-2(R)-((4-(2-(4-morpholinyl)ethoxy)phenyl)methyl)-4(S)-hydroxy-5-(1-(4-(3-phenylpropyl)-2(S)-N′-(t-burylcarboxamido))-piperazinyl)-pentaneamide,
N-(2(R)-hydroxy-1(S)-indanyl)-2(R)-((4-(2-(4-morpholinyl)ethoxy)phenyl)methyl)-4(S)-amino-5-(1-(4carbobenzyloxy-2(S)-N′-(t-butylcarboxamido)piperazinyl)-pentaneamide,
N-(2(R)-hydroxy-1(S)-indanyl)-2(R)-((4-((2-hydroxy)-ethoxy)phenyl)methyl)-4(S)-hydroxy-5-(1-(N′-(t-butyl)-4(S)-phenoxyprolineamid)yl)-pentaneamide,
N-(2(R)-hydroxy-1(S)-indanyl)-2(R)-((4-((2-hydroxy)ethoxy)phenyl)methyl)-4(S)-hydroxy-5-(1-(N′-t-butyl-4(S)-2-naphthyloxy-prolineamid)yl)-pentaneamide,
N-(2(R)-hydroxy-1(S)-indanyl)-2(R)-((4-((2-hydroxy)-ethoxy)phenyl)methyl)-4(S)-hydroxy-5-(1-(N′-t-butyl4(S)-1-naphthyloxy-prolineamid)yl)-pentaneamide,
N-(2(R)-hydroxy-1(S)-indanyl)-2(R)-((4-((2-hydroxy)ethoxy)phenyl)methyl)-4(S)-amino-5-(2-(3(S)-N′-(t-butylcarboxamido)-(4aS,8aS)-decahydroisoquinoline)-yl)pentaneamide,
N-(2(R)-hydroxy-1(S)-indanyl)-2(R)-((4-((2-hydroxy)-ethoxy)phenyl)methyl)-4(S)-hydroxy-5-(1-(4-(3-phenylpropionyl)-2(S)-N′-(t-butylcarboxamido)-piperazinyl))pentaneamide,
N-(2(R)-hydroxy-1(S)-indanyl)-2(R)-((4-((2-hydroxy)-ethoxy)phenyl)methyl)-4(S)-hydroxy-5-(1-(4-benzoyl2(S)-N′-(t-butylcarboxamido)-piperazinyl))-pentaneamide,
N-(2(R)-hydroxy-1(S)-indanyl)-2(R)-((4-((2-hydroxy)-ethoxy)phenyl)methyl)-4(S)-hydroxy-5-(1-(4-(3-phenylpropyl)-2(S)-N′-(t-butylcarboxamido))-piperazinyl)-pentaneamide,
N-(2(R)-hydroxy-1(S)-indanyl)-2(R)-((4-((2-hydroxy)-ethoxy)phenyl)methyl)-4(S)-amino-5-(1-(4-carbobenzyloxy-2(S)-N′-(t-butylcarboxamido)-piperazinyl))-pentaneamide,
N-(4(S)-3,4-dihydro-1H-2,2-dioxobenzothiopyranyl)-2(R)-phenylmethyl-4(S)-hydroxy-5-(1-(N′-(t-butyl)-4(S)-phenoxyprolineamid)yl)-pentaneamide,
N-(4(S)-3,4-dihydro-1H-2,2-dioxobenzothiopyranyl)-2-(R)-phenylmethyl-4(S)-hydroxy-5-(1-(N′-t-butyl-4(S)-2-naphthyloxy-prolineamid)yl)-pentaneamide,
N-(4(S)-3,4-dihydro-1H-2,2-dioxobenzothiopyranyl)-2-(R)-phenylmethyl-4(S)-hydroxy-5-(1-(N′-t-butyl-4(S)-1-naphthyloxy-prolineamid)yl)-pentaneamide,
N-(4(S)-3,4-dihydro-1H-2,2-dioxobenzothiopyranyl)-2-(R)-phenylmethyl-4(S)-amino-5-(2-(3(S)-N′-(t-butylcarboxamido)-(4aS,8aS)-decahydroisoquinoline)yl)pentaneamide,
N-(4(S)-3,4-dihydro-1H-2,2-dioxobenzothiopyranyl)-2-(R)-phenylmethyl-4(S)-hydroxy-5-(1-(4-(3-phenylpropionyl)-2(S)-N′-(t-butylcarboxamido)-piperazinyl))pentaneamide,
N-(4(S)-3,4-dihydro-1H-2,2-dioxobenzothiopyranyl)-2-(R)-phenylmethyl-4(S)-hydroxy-5-(1-(4-benzoyl-2(S)-N′-(t-butylcarboxamido)-piperazinyl))-pentaneamide,
N-(4(S)-3,4-dihydro-1H-2,2-dioxobenzothiopyranyl)-2-(R)-phenylmethyl-4(S)-hydroxy-5-(1-(4-(3-phenylpropyl)-2(S)-N′-(t-butylcarboxamido))-piperazinyl)pentaneamide, or
(4(S)-3,4-dihydro-1H-2,2-dioxobenzothiopyranyl)-2-(R)-phenylmethyl-4(S)-amino-5-(l-(4-carbobenzyloxy-2-(S)N′-(t-butylcarboxamido)-piperazinyl))-pentaneamide.

The compounds employed in the methods of the present invention, may have asymmetric centers and occur as racemates, racemic mixtures and as individual diastereomers, or enantiomers with all isomeric forms being included in the present invention.

When any variable (e.g., aryl, heterocycle, R, R¹, R², A⁻, n, Z, etc.) occurs more than one time in any constituent or in formula I, its definition on each occurrence is independent of its definition at every other occurrence. Also, combinations of substituents and/or variables are permissible only if such combinations result in stable compounds.

Definitions

The compounds employed in the methods of this invention are identified in two ways: by descriptive names and by reference to structures having various chemical moieties. The following terms may also be used and are defined below.

The term “modulating” refers to the ability of a compound to at least partially block the active site of the beta amyloid converting enzyme, thereby decreasing, or inhibiting the turnover rate of the enzyme.

As used herein except where noted, “alkyl” is intended to include both branched- and straight-chain saturated aliphatic hydrocarbon groups having the specified number of carbon atoms (Me is methyl, Et is ethyl, Pr is propyl, Bu is butyl); “alkoxy” represents an alkyl group of indicated number of carbon atoms attached through an oxygen bridge; and “cycloalkyl” is intended to include saturated ring groups, such as cyclopropyl, cyclobutyl, cyclopentyl, cyclohexyl (Cyh) and cycloheptyl. “Alkenyl” is intended to include hydrocarbon groups of either a straight or branched configuration with one or more carbon-carbon double bonds which may occur in any stable point along the chain, such as ethenyl, propenyl, butenyl, pentenyl, and the like. “Alkyny” is intended to include hydrocarbon groups of either a straight or branched configuration with one or more carbon-carbon triple bonds which may occur in any stable point along the chain, such as ethynyl, propynyl, butynyl, pentynyl, and the like. “Halo”, as used herein, means fluoro, chloro, bromo and iodo; and “counterion” is used to represent a small, single negatively-charged species, such as chloride, bromide, hydroxide, acetate, trifluroacetate, perchlorate, nitrate, benzoate, maleate, tartrate, hemitartrate, benzene sulfonate, and the like.

As used herein, with exceptions as noted, “aryl” is intended to mean phenyl (Ph) or naphthyl. “Carbocyclic” is intended to mean any stable 5- to 7-membered carbon ring or 7- to 10-membered bicyclic carbon ring any ring of which may be saturated or unsaturated.

The term heterocycle or heterocyclic, as used herein except where noted, represents a stable 5- to 7-membered mono- or bicyclic or stable 7- to 10-membered bicyclic heterocyclic ring system any ring of which may be saturated or unsaturated, and which consists of carbon atoms and from one to three heteroatoms selected from the group consisting of N, O and S, and wherein the nitrogen and sulfur heteroatoms may optionally be oxidized, and the nitrogen heteroatom may optionally be quaternized, and including any bicyclic group in which any of the above-defined heterocyclic rings is fused to a benzene ring. The heterocyclic ring may be attached at any heteroatom or carbon atom which results in the creation of a stable structure. Examples of such heterocyclic elements include piperidinyl, piperazinyl, 2-oxopiperazinyl, 2-oxopiperidinyl, 2-oxopyrrolodinyl, 2-oxoazepinyl, azepinyl, pyrrolyl, 4-piperidonyl, pyrrolidinyl, pyrazolyl, pyrazolidinyl, imidazolyl, imidazolinyl, imidazolidinyl, pyridyl, pyrazinyl, pyrimidinyl, pyridazinyl, oxazolyl, oxazolidinyl, isoxazolyl, isoxazolidinyl, morpholinyl, thiazolyl, thiazolidinyl, isothiazolyl, quinuclidinyl, isothiazolidinyl, indolyl, quinolinyl, isoquinolinyl, benzimidazolyl, thiadiazoyl, benzopyranyl, benzothiazolyl, benzoxazolyl, furyl, tetrahydrofuryl, tetrahydropyranyl, thienyl, benzothienyl, thiamorpholinyl, thiamorpholinyl sulfoxide, thiamorpholinyl sulfone, and oxadiazolyl. Morpholino is the same as morpholinyl.

The pharmaceutically-acceptable salts of the compounds of Formula I (in the form of water- or oil-soluble or dispersible products) include the conventional non-toxic salts or the quaternary ammonium salts which are formed, e.g., from inorganic or organic acids or bases. Examples of such acid addition salts include acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, glucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, pamoate, pectinate, persulfate, 3-phenylpropionate, picrate, pivalate, propionate, succinate, tartrate, thiocyanate, tosylate, and undecanoate. Base salts include ammonium salts, alkali metal salts such as sodium and potassium salts, alkaline earth metal salts such as calcium and magnesium salts, salts with organic bases such as dicyclohexylamine salts, N-methyl-D-glucamine, and salts with amino acids such as arginine, lysine, and so forth. Also, the basic nitrogen-containing groups may be quaternized with such agents as lower alkyl halides, such as methyl, ethyl, propyl, and butyl chloride, bromides and iodides; dialkyl sulfates like dimethyl, diethyl, dibutyl; and diamyl sulfates, long chain halides such as decyl, lauryl, myristyl and stearyl chlorides, bromides and iodides, aralkyl halides like benzyl and phenethyl bromides and others. Other pharmaceutically acceptable salts include the sulfate salt ethanolate and sulfate salts.

Some abbreviations that may appear in this application are as follows:

ABBREVIATIONS

| Designation | |
|---|---|
| | Protecting Group |
| BOC (Boc) | t-butyloxycarbonyl |
| CBZ (Cbz) | benzyloxycarbonyl (carbo-benzoxy) |
| TBS (TBDMS) | t-butyl-dimethylsilyl |
| | Activating Group |
| HBT(HOBT or HOBt) | 1-hydroxybenzotriazole |
| | hydrate |
| | Coupling Reagent |
| BOP reagent | benzotriazol-1-yloxytris- |
| | (dimethylamino)phosphonium |
| | hexafluorophosphate |
| BOP-C1 | bis(2-oxo-3-oxazolidinyl) |
| | phosphinic chloride |
| EDC | 1-ethyl-3-(3-dimethyl-aminopropyl) |
| | Carbodiimide hydrochloride |
| | Other |
| (BOC)₂O (BOC₂O) | di-t-butyl dicarbonate |
| n-Bu₄N⁺F⁻ | tetrabutyl ammonium fluoride |
| nBuLi (n-Buli) | n-butyllithium |
| DMF | dimethylformamide |
| Et₃N | triethylamine |
| EtOAc | ethyl acetate |
| TFA | trifluoroacetic acid |
| DMAP | dimethylaminopyridine |
| DME | dimethoxyethane |
| LDA | lithium diisopropylamide |
| THF | tetrahydrofuran |
| | Amino Acid |
| Ile | L-isoleucine |
| Val | L-valine |

Administration and Compositions

In clinical practice the compounds employed in the methods of the invention will normally be administered orally, rectally, or by injection, in the form of pharmaceutical preparations comprising the active ingredient either as a free acid or as a pharmaceutically acceptable non-toxic, base addition salt, such as of the types listed above in association with a pharmaceutically acceptable carrier. The use and administration to a patient to be treated in the clinic would be readily apparent to a physician or pharmacist or ordinary skill in the art.

The present invention also provides pharmaceutical compositions which comprise one or more of the compounds of formula I above formulated together with one or more non-toxic pharmaceutically acceptable carriers. The pharmaceutical compositions may be specially formulated for oral administration in solid or liquid form, for parenteral injection, or for rectal administration.

The pharmaceutical compositions employed in the methods of this invention can be administered to humans and other animals orally, rectally, parenterally (i.e., intravenously, intramuscularly, or subcutaneously), intracisternally, intravaginally, intraperitoneally, topically (as by powders, ointments, or drops), bucally, or as an oral or nasal spray.

Pharmaceutical compositions for use in the methods of this invention for parenteral injection comprise pharmaceutically acceptable sterile aqueous or nonaqueous solutions, dispersions, suspensions or emulsions as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol, and the like), and suitable mixtures thereof, vegetable oils (such as olive oil), and injectable organic esters such as ethyl oleate. Proper fluidity can be maintained, for example, by the use of coating materials such as lecithin, by the maintenance of the required particle size in the case of dispersions, and by the use of surfactants.

These compositions may also contain adjuvants such as preservative, wetting agents, emulsifying agents, and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents such as sugars, sodium chloride, and the like. Prolonged absorption of the injectable pharmaceutical form may be brought about by the inclusion of agents which delay absorption such as aluminum monostearate and gelatin.

If desired and for more effective distribution, the compounds can be incorporated into slow release or targeted delivery systems such as polymer matrices, liposomes, and microspheres.

The injectable formulations can be sterilized, for example, by filtration through a bacterial-retaining filter, or by incorporating sterilizing agents in the form of sterile solid compositions which can be dissolved or dispersed in sterile water or other sterile injectable medium just prior to use.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alignates, gelatin, polyvinylpyrrolidone, sucrose, and acacia, c) humectants such as glycerol, d) disintegrating agents such as agar-agar, calcium carbonate, potato ortapioca starch, alginic acid, certain silicates, and sodium carbonate, e) solution retarding agents such as paraffin, f) absorption accelerators such as quaternaryammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in sort and hard-filled gelatin capsules using such excipients as lactose or milk sugar as well as high molecular weight polyethylene glycols and the like.

The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions which can be used include polymeric substances and waxes.

The active compounds can also be in microencapsulated form, if appropriate, with one or more of the above-mentioned excipients.

Liquid dosage forms for oral administration include pharmaceutically acceptable emulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethyl formamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, and perfuming agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite agar-agar, and tragacanth, and mixtures thereof.

Compositions for rectal or vaginal administration are preferably suppositories which can be prepared by mixing the compounds of this invention with suitable nonirritating excipients or carriers such as cocoa butter, polyethylene glycol or a suppository wax which are solid at room temperature but liquid at body temperature and therefore melt in the rectum or vaginal cavity and release the active compound.

Dosage forms for topical administration of a compound of this invention include powders, sprays, ointments and inhalants. The active compound is mixed under sterile conditions with a pharmaceutically acceptable carrier and any needed preservatives, buffers, or propellants which may be required. Ophthalmic formulations, eye ointments, powders and solutions are also contemplated as being within the scope of this invention.

Actual dosage levels of active ingredients in the pharmaceutical compositions of this invention may be varied so as to obtain an amount of the active compound(s) that is effective to achieve the desired therapeutic response for a particular patient, compositions, and mode of administration. The selected dosage level will depend upon the activity of the particular compound, the route of administration, the severity of the condition being treated, and the condition and prior medical history of the patient being treated. However, it is within the skill of the art to start doses of the compound at levels lower than required to achieve the desired therapeutic effect and to gradually increase the dosage until the desired effect is achieved. Generally dosage levels of about 0.1 to about 200, more preferably of about 0.5 to about 150, and most preferably about 1 to about 125 mg of active compound per kilogram of body weight per day are administered orally to a mammalian patient. If desired, the effective daily dose may be divided into multiple doses for purposes of administration, e.g., two to four separate doses per day.

Methods of the Invention

The compounds of the invention, and pharmaceutically acceptable salts thereof, are useful for treating humans or animals suffering from a condition characterized by a pathological form of beta-amyloid peptide, such as beta-amyloid plaques, and for helping to prevent or delay the onset of such a condition. For example, the compounds are useful for treating Alzheimer's disease, for helping prevent or delay the onset of Alzheimer's disease, for treating subjects with MCI (mild cognitive impairment) and preventing or delaying the onset of Alzheimer's disease in those who would progress from MCI to AD, for treating Down's syndrome, for treating humans who have Hereditary Cerebral Hemorrhage with Amyloidosis of the Dutch-Type, for treating cerebral amyloid angiopathy and preventing its potential consequences, i.e. single and recurrent lobal hemorrhages, for treating other degenerative dementias, including dementias of mixed vascular and degenerative origin, dementia associated with Parkinson's disease, frontotemporal dementias with parkinsonism (FTDP), dementia associated with progressive supranuclear palsy, dementia associated with cortical basal degeneration, and diffuse Lewy body type Alzheimer's disease. The compounds and compositions of the invention are particularly useful for treating, preventing, or slowing the progression of Alzheimer's disease. When treating or preventing these diseases, the compounds of the invention can either be used individually or in combination, as is best for the subject or subject.

With regard to these diseases, the term “treating” means that compounds of the invention can be used in humans with existing disease. The compounds of the invention will not necessarily cure the subject who has the disease but will delay or slow the progression or prevent further progression of the disease thereby giving the individual a more useful life span.

The term “preventing” means that that if the compounds of the invention are administered to those who do not now have the disease but who would normally develop the disease or be at increased risk for the disease, they will not develop the disease. In addition, “preventing” also includes delaying the development of the disease in an individual who will ultimately develop the disease or would be at risk for the disease due to age, familial history, genetic or chromosomal abnormalities, and/or due to the presence of one or more biological markers for the disease, such as a known genetic mutation of APP or APP cleavage products in brain tissues or fluids. By delaying the onset of the disease, compounds of the invention have prevented the individual from getting the disease during the period in which the individual would normally have gotten the disease or reduce the rate of development of the disease or some of its effects but for the administration of compounds of the invention up to the time the individual ultimately gets the disease. Preventing also includes administration of the compounds of the invention to those individuals thought to be predisposed to the disease.

In a preferred aspect, the compounds of the invention are useful for slowing the progression of disease symptoms.

In another preferred aspect, the compounds of the invention are useful for preventing the further progression of disease symptoms.

In treating or preventing the above diseases, the compounds of the invention are administered in a therapeutically effective amount. The therapeutically effective amount will vary depending on the particular compound used and the route of administration, as is known to those skilled in the art.

In treating a subject displaying any of the diagnosed above conditions a physician may administer a compound of the invention immediately and continue administration indefinitely, as needed. In treating subjects who are not diagnosed as having Alzheimer's disease, but who are believed to be at substantial risk for Alzheimer's disease, the physician should preferably start treatment when the subject first experiences early pre-Alzheimer's symptoms such as, memory or cognitive problems associated with aging. In addition, there are some subjects who may be determined to be at risk for developing Alzheimer's through the detection of a genetic marker such as APOE4 or other biological indicators that are predictive for Alzheimer's disease. In these situations, even though the subject does not have symptoms of the disease, administration of the compounds of the invention may be started before symptoms appear, and treatment may be continued indefinitely to prevent or delay the onset of the disease.

Definitions

APP, amyloid precursor protein, is defined as any APP polypeptide, including APP variants, mutations, and isoforms, for example, as disclosed in U.S. Pat. No. 5,766,846.

A beta, amyloid beta peptide, is defined as any peptide resulting from beta-secretase mediated cleavage of APP, including peptides of 39, 40, 41, 42, and 43 amino acids, and extending from the beta-secretase cleavage site to amino acids 39, 40, 41, 42, or 43.

Beta-secretase (BACE1, Asp2, Memapsin 2) is an aspartyl protease that mediates cleavage of APP at the amino-terminal edge of A beta. Human beta-secretase is described, for example, in WO00/17369.

Pharmaceutically acceptable refers to those properties and/or substances that are acceptable to the patient from a pharmacological/toxicological point of view and to the manufacturing pharmaceutical chemist from a physical/chemical point of view regarding composition, formulation, stability, patient acceptance and bioavailability.

A therapeutically effective amount is defined as an amount effective to reduce or lessen at least one symptom of the disease being treated or to reduce or delay onset of one or more clinical markers or symptoms of the disease.

Dosage Forms and Amounts

The compounds of the invention can be administered orally, parenterally, (IV, IM, depo-IM, SQ, and depo SQ), sublingually, intranasally (inhalation), intrathecally, topically, or rectally. Dosage forms known to those of skill in the art are suitable for delivery of the compounds of the invention.

Compositions are provided that contain therapeutically effective amounts of the compounds of the invention. The compounds are preferably formulated into suitable pharmaceutical preparations such as tablets, capsules, or elixirs for oral administration or in sterile solutions or suspensions for parenteral administration. Typically the compounds described above are formulated into pharmaceutical compositions using techniques and procedures well known in the art.

About 1 to 500 mg of a compound or mixture of compounds of the invention or a physiologically acceptable salt or ester is compounded with a physiologically acceptable vehicle, carrier, excipient, binder, preservative, stabilizer, flavor, etc., in a unit dosage form as called for by accepted pharmaceutical practice. The amount of active substance in those compositions or preparations is such that a suitable dosage in the range indicated is obtained. The compositions are preferably formulated in a unit dosage form, each dosage containing from about 2 to about 100 mg, more preferably about 10 to about 30 mg of the active ingredient. The term “unit dosage from” refers to physically discrete units suitable as unitary dosages for human subjects and other mammals, each unit containing a predetermined quantity of active material calculated to produce the desired therapeutic effect, in association with a suitable pharmaceutical excipient.

To prepare compositions, one or more compounds of the invention are mixed with a suitable pharmaceutically acceptable carrier. Upon mixing or addition of the compound(s), the resulting mixture may be a solution, suspension, emulsion, or the like. Liposomal suspensions may also be suitable as pharmaceutically acceptable carriers. These may be prepared according to methods known to those skilled in the art. The form of the resulting mixture depends upon a number of factors, including the intended mode of administration and the solubility of the compound in the selected carrier or vehicle. The effective concentration is sufficient for lessening or ameliorating at least one symptom of the disease, disorder, or condition treated and may be empirically determined.

Pharmaceutical carriers or vehicles suitable for administration of the compounds provided herein include any such carriers known to those skilled in the art to be suitable for the particular mode of administration. In addition, the active materials can also be mixed with other active materials that do not impair the desired action, or with materials that supplement the desired action, or have another action. The compounds may be formulated as the sole pharmaceutically active ingredient in the composition or may be combined with other active ingredients.

Where the compounds exhibit insufficient solubility, methods for solubilizing may be used. Such methods are known and include, but are not limited to, using cosolvents such as dimethylsulfoxide (DMSO), using surfactants such as Tween®, and dissolution in aqueous sodium bicarbonate. Derivatives of the compounds, such as salts or prodrugs may also be used in formulating effective pharmaceutical compositions.

The concentration of the compound is effective for delivery of an amount upon administration that lessens or ameliorates at least one symptom of the disorder for which the compound is administered. Typically, the compositions are formulated for single dosage administration.

The compounds of the invention may be prepared with carriers that protect them against rapid elimination from the body, such as time-release formulations or coatings. Such carriers include controlled release formulations, such as, but not limited to, microencapsulated delivery systems. The active compound is included in the pharmaceutically acceptable carrier in an amount sufficient to exert a therapeutically useful effect in the absence of undesirable side effects on the subject treated. The therapeutically effective concentration may be determined empirically by testing the compounds in known in vitro and in vivo model systems for the treated disorder.

The compounds and compositions of the invention can be enclosed in multiple or single dose containers. The enclosed compounds and compositions can be provided in kits, for example, including component parts that can be assembled for use. For example, a compound inhibitor in lyophilized form and a suitable diluent may be provided as separated components for combination prior to use. A kit may include a compound inhibitor and a second therapeutic agent for co-administration. The inhibitor and second therapeutic agent may be provided as separate component parts. A kit may include a plurality of containers, each container holding one or more unit dose of the compound of the invention. The containers are preferably adapted for the desired mode of administration, including, but not limited to tablets, gel capsules, sustained-release capsules, and the like for oral administration; depot products, pre-filled syringes, ampoules, vials, and the like for parenteral administration; and patches, medipads, creams, and the like for topical administration.

The concentration of active compound in the drug composition will depend on absorption, inactivation, and excretion rates of the active compound, the dosage schedule, and amount administered as well as other factors known to those of skill in the art.

The active ingredient may be administered at once, or may be divided into a number of smaller doses to be administered at intervals of time. It is understood that the precise dosage and duration of treatment is a function of the disease being treated and may be determined empirically using known testing protocols or by extrapolation from in vivo or in vitro test data. It is to be noted that concentrations and dosage values may also vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed compositions.

If oral administration is desired, the compound should be provided in a composition that protects it from the acidic environment of the stomach. For example, the composition can be formulated in an enteric coating that maintains its integrity in the stomach and releases the active compound in the intestine. The composition may also be formulated in combination with an antacid or other such ingredient.

Oral compositions will generally include an inert diluent or an edible carrier and may be compressed into tablets or enclosed in gelatin capsules. For the purpose of oral therapeutic administration, the active compound or compounds can be incorporated with excipients and used in the form of tablets, capsules, or troches. Pharmaceutically compatible binding agents and adjuvant materials can be included as part of the composition.

The tablets, pills, capsules, troches, and the like can contain any of the following ingredients or compounds of a similar nature: a binder such as, but not limited to, gum tragacanth, acacia, corn starch, or gelatin; an excipient such as microcrystalline cellulose, starch, or lactose; a disintegrating agent such as, but not limited to, alginic acid and corn starch; a lubricant such as, but not limited to, magnesium stearate; a gildant, such as, but not limited to, colloidal silicon dioxide; a sweetening agent such as sucrose or saccharin; and a flavoring agent such as peppermint, methyl salicylate, or fruit flavoring.

When the dosage unit form is a capsule, it can contain, in addition to material of the above type, a liquid carrier such as a fatty oil. In addition, dosage unit forms can contain various other materials, which modify the physical form of the dosage unit, for example, coatings of sugar and other enteric agents. The compounds can also be administered as a component of an elixir, suspension, syrup, wafer, chewing gum or the like. A syrup may contain, in addition to the active compounds, sucrose as a sweetening agent and certain preservatives, dyes and colorings, and flavors.

The active materials can also be mixed with other active materials that do not impair the desired action, or with materials that supplement the desired action.

Solutions or suspensions used for parenteral, intradermal, subcutaneous, or topical application can include any of the following components: a sterile diluent such as water for injection, saline solution, fixed oil, a naturally occurring vegetable oil such as sesame oil, coconut oil, peanut oil, cottonseed oil, and the like, or a synthetic fatty vehicle such as ethyl oleate, and the like, polyethylene glycol, glycerine, propylene glycol, or other synthetic solvent; antimicrobial agents such as benzyl alcohol and methyl parabens; antioxidants such as ascorbic acid and sodium bisulfite; chelating agents such as ethylenediaminetetraacetic acid (EDTA); buffers such as acetates, citrates, and phosphates; and agents for the adjustment of tonicity such as sodium chloride and dextrose. Parenteral preparations can be enclosed in ampoules, disposable syringes, or multiple dose vials made of glass, plastic, or other suitable material. Buffers, preservatives, antioxidants, and the like can be incorporated as required.

Where administered intravenously, suitable carriers include physiological saline, phosphate buffered saline (PBS), and solutions containing thickening and solubilizing agents such as glucose, polyethylene glycol, polypropyleneglycol, and mixtures thereof. Liposomal suspensions including tissue-targeted liposomes may also be suitable as pharmaceutically acceptable carriers. These may be prepared according to methods known for example, as described in U.S. Pat. No. 4,522,811.

The active compounds may be prepared with carriers that protect the compound against rapid elimination from the body, such as time-release formulations or coatings. Such carriers include controlled release formulations, such as, but not limited to, implants and microencapsulated delivery systems, and biodegradable, biocompatible polymers such as collagen, ethylene vinyl acetate, polyanhydrides, polyglycolic acid, polyorthoesters, polylactic acid, and the like. Methods for preparation of such formulations are known to those skilled in the art.

The compounds of the invention can be administered orally, parenterally (IV, IM, depo-IM, SQ, and depo-SQ), sublingually, intranasally (inhalation), intrathecally, topically, or rectally. Dosage forms known to those skilled in the art are suitable for delivery of the compounds of the invention.

Compounds of the invention may be administered enterally or parenterally. When administered orally, compounds of the invention can be administered in usual dosage forms for oral administration as is well known to those skilled in the art. These dosage forms include the usual solid unit dosage forms of tablets and capsules as well as liquid dosage forms such as solutions, suspensions, and elixirs. When the solid dosage forms are used, it is preferred that they be of the sustained release type so that the compounds of the invention need to be administered only once or twice daily.

The oral dosage forms are administered to the subject 1, 2, 3, or 4 times daily. It is preferred that the compounds of the invention be administered either three or fewer times, more preferably once or twice daily. Hence, it is preferred that the compounds of the invention be administered in oral dosage form. It is preferred that whatever oral dosage form is used, that it be designed so as to protect the compounds of the invention from the acidic environment of the stomach. Enteric coated tablets are well known to those skilled in the art. In addition, capsules filled with small spheres each coated to protect from the acidic stomach, are also well known to those skilled in the art.

When administered orally, an administered amount therapeutically effective to inhibit beta-secretase activity, to inhibit A beta production, to inhibit A beta deposition, or to treat or prevent AD is from about 0.1 mg/day to about 1,000 mg/day. It is preferred that the oral dosage is from about 1 mg/day to about 100 mg/day. It is more preferred that the oral dosage is from about 5 mg/day to about 50 mg/day. It is understood that while a subject may be started at one dose, that dose may be varied over time as the subject's condition changes.

Compounds of the invention may also be advantageously delivered in a nano crystal dispersion formulation. Preparation of such formulations is described, for example, in U.S. Pat. No. 5,145,684. Nano crystalline dispersions of HIV protease inhibitors and their method of use are described in U.S. Pat. No. 6,045,829. The nano crystalline formulations typically afford greater bioavailability of drug compounds.

The compounds of the invention can be administered parenterally, for example, by IV, IM, depo-IM, SC, or depo-SC. When administered parenterally, a therapeutically effective amount of about 0.5 to about 100 mg/day, preferably from about 5 to about 50 mg daily should be delivered. When a depot formulation is used for injection once a month or once every two weeks, the dose should be about 0.5 mg/day to about 50 mg/day, or a monthly dose of from about 15 mg to about 1,500 mg. In part because of the forgetfulness of the subjects with Alzheimer's disease, it is preferred that the parenteral dosage form be a depo formulation.

The compounds of the invention can be administered sublingually. When given sublingually, the compounds of the invention should be given one to four times daily in the amounts described above for IM administration.

The compounds of the invention can be administered intranasally. When given by this route, the appropriate dosage forms are a nasal spray or dry powder, as is known to those skilled in the art. The dosage of the compounds of the invention for intranasal administration is the amount described above for IM administration.

The compounds of the invention can be administered intrathecally. When given by this route the appropriate dosage form can be a parenteral dosage form as is known to those skilled in the art. The dosage of the compounds of the invention for intrathecal administration is the amount described above for IM administration.

The compounds of the invention can be administered topically. When given by this route, the appropriate dosage form is a cream, ointment, or patch. Because of the amount of the compounds of the invention to be administered, the patch is preferred. When administered topically, the dosage is from about 0.5 mg/day to about 200 mg/day. Because the amount that can be delivered by a patch is limited, two or more patches may be used. The number and size of the patch is not important, what is important is that a therapeutically effective amount of the compounds of the invention be delivered as is known to those skilled in the art. The compounds of the invention can be administered rectally by suppository as is known to those skilled in the art. When administered by suppository, the therapeutically effective amount is from about 0.5 mg to about 500 mg.

The compounds of the invention can be administered by implants as is known to those skilled in the art. When administering a compound of the invention by implant, the therapeutically effective amount is the amount described above for depot administration.

The invention here is the new compounds of the invention and new methods of using the compounds of the invention. Given a particular compound of the invention and a desired dosage form, one skilled in the art would know how to prepare and administer the appropriate dosage form. The compounds of the invention are used in the same manner, by the same routes of administration, using the same pharmaceutical dosage forms, and at the same dosing schedule as described above, for preventing disease or treating subjects with MCI (mild cognitive impairment) and preventing or delaying the onset of Alzheimer's disease in those who would progress from MCI to AD, for treating or preventing Down's syndrome, for treating humans who have Hereditary Cerebral Hemorrhage with Amyloidosis of the Dutch-Type, for treating cerebral amyloid angiopathy and preventing its potential consequences, i.e. single and recurrent lobar hemorrhages, for treating other degenerative dementias, including dementias of mixed vascular and degenerative origin, dementia associated with Parkinson's disease, frontotemporal dementias with parkinsonism (FTDP), dementia associated with progressive supranuclear palsy, dementia associated with cortical basal degeneration, and diffuse Lewy body type of Alzheimer's disease.

The compounds of the invention can be used with each other or with other agents used to treat or prevent the conditions listed above. Such agents include gamma-secretase inhibitors, anti-amyloid vaccines and pharmaceutical agents such as donepezil hydrochloride (ARICEPT Tablets), tacrine hydrochloride (COGNEX Capsules) or other acetylcholine esterase inhibitors and with direct or indirectneurotropic agents of the future.

In addition, the compounds of the invention can also be used with inhibitors of P-glycoproten (P-gp). The use of P-gp inhibitors is known to those skilled in the art. See for example, Cancer Research, 53, 4595-4602 (1993), Clin. Cancer Res., 2, 7-12 (1996), Cancer Research, 56, 4171-4179 (1996), International Publications WO99/64001 and WO01/10387. The important thing is that the blood level of the P-gp inhibitor be such that it exerts its effect in inhibiting P-gp from decreasing brain blood levels of the compounds of the invention. To that end the P-gp inhibitor and the compounds of the invention can be administered at the same time, by the same or different route of administration, or at different times. The important thing is not the time of administration but having an effective blood level of the P-gp inhibitor.

Suitable P-gp inhibitors include cyclosporin A, verapamil, tamoxifen, quinidine, Vitamin E-TGPS, ritonavir, megestrol acetate, progesterone, rapamycin, 10,11-methanodibenzosuberane, phenothiazines, acridine derivatives such as GF120918, FK506, VX-710, LY335979, PSC-833, GF-102,918 and other steroids. It is to be understood that additional agents will be found that do the same function and are also considered to be useful.

The P-gp inhibitors can be administered orally, parenterally, (IV, IM, IM-depo, SQ, SQ-depo), topically, sublingually, rectally, intranasally, intrathecally and by implant.

The therapeutically effective amount of the P-gp inhibitors is from about 0.1 to about 300 mg/kg/day, preferably about 0.1 to about 150 mg/kg daily. It is understood that while a subject may be started on one dose, that dose may have to be varied over time as the subject's condition changes.

When administered orally, the P-gp inhibitors can be administered in usual dosage forms for oral administration as is known to those skilled in the art. These dosage forms include the usual solid unit dosage forms of tablets and capsules as well as liquid dosage forms such as solutions, suspensions and elixirs. When the solid dosage forms are used, it is preferred that they be of the sustained release type so that the P-gp inhibitors need to be administered only once or twice daily. The oral dosage forms are administered to the subject one through four times daily. It is preferred that the P-gp inhibitors be administered either three or fewer times a day, more preferably once or twice daily. Hence, it is preferred that the P-gp inhibitors be administered in solid dosage form and further it is preferred that the solid dosage form be a sustained release form which permits once or twice daily dosing. It is preferred that what ever dosage form is used, that it be designed so as to protect the P-gp inhibitors from the acidic environment of the stomach. Enteric coated tablets are well known to those skilled in the art. In addition, capsules filled with small spheres each coated to protect from the acidic stomach, are also well known to those skilled in the art.

In addition, the P-gp inhibitors can be administered parenterally. When administered parenterally they can be administered IV, IM, depo-IM, SQ or depo-SQ. The P-gp inhibitors can be given sublingually. When given sublingually, the P-gp inhibitors should be given one thru four times daily in the same amount as for IM administration.

The P-gp inhibitors can be given intranasally. When given by this route of administration, the appropriate dosage forms are a nasal spray or dry powder as is known to those skilled in the art. The dosage of the P-gp inhibitors for intranasal administration is the same as for IM administration.

The P-gp inhibitors can be given intrathecally. When given by this route of administration the appropriate dosage form can be a parenteral dosage form as is known to those skilled in the art.

The P-gp inhibitors can be given topically. When given by this route of administration, the appropriate dosage form is a cream, ointment or patch. Because of the amount of the P-gp inhibitors needed to be administered the path is preferred. However, the amount that can be delivered by a patch is limited. Therefore, two or more patches may be required. The number and size of the patch is not important, what is important is that a therapeutically effective amount of the P-gp inhibitors be delivered as is known to those skilled in the art. The P-gp inhibitors can be administered rectally by suppository as is known to those skilled in the art.

The P-gp inhibitors can be administered by implants as is known to those skilled in the art.

There is nothing novel about the route of administration nor the dosage forms for administering the P-gp inhibitors. Given a particular P-gp inhibitor, and a desired dosage form, one skilled in the art would know how to prepare the appropriate dosage form for the P-gp inhibitor.

The compounds employed in the methods of the invention can be used in combination, with each other or with other therapeutic agents or approaches used to treat or prevent the conditions listed above. Such agents or approaches include: acetylcholine esterase inhibitors such as tacrine (tetrahydroaminoacridine, marketed as COGNEX®), donepezil hydrochloride, (marketed as Aricept® and rivastigmine (marketed as Exelon®); gamma-secretase inhibitors; anti-inflammatory agents such as cyclooxygenase II inhibitors; anti-oxidants such as Vitamin E and ginkolides; immunological approaches, such as, for example, immunization with A beta peptide or administration of anti-A beta peptide antibodies; statins; and direct or indirect neurotropic agents such as Cerebrolysin®, AIT-082 (Emilieu, 2000, Arch. Neurol. 57:454), and other neurotropic agents of the future.

It should be apparent to one skilled in the art that the exact dosage and frequency of administration will depend on the particular compounds employed in the methods of the invention administered, the particular condition being treated, the severity of the condition being treated, the age, weight, general physical condition of the particular subject, and other medication the individual may be taking as is well known to administering physicians who are skilled in this art.

Inhibition of APP Cleavage

The compounds of the invention inhibit cleavage of APP between Met595 and Asp596 numbered for the APP695 isoform, or a mutant thereof, or at a corresponding site of a different isoform, such as APP751 or APP770, or a mutant thereof (sometimes referred to as the “beta secretase site”). While not wishing to be bound by a particular theory, inhibition of beta-secretase activity is thought to inhibit production of beta amyloid peptide (A beta). Inhibitory activity is demonstrated in one of a variety of inhibition assays, whereby cleavage of an APP substrate in the presence of a beta-secretase enzyme is analyzed in the presence of the inhibitory compound, under conditions normally sufficient to result in cleavage at the beta-secretase cleavage site. Reduction of APP cleavage at the beta-secretase cleavage site compared with an untreated or inactive control is correlated with inhibitory activity. Assay systems that can be used to demonstrate efficacy of the compound inhibitors of the invention are known. Representative assay systems are described, for example, in U.S. Pat. Nos. 5,942,400, 5,744,346, as well as in the Examples below.

The enzymatic activity of beta-secretase and the production of A beta can be analyzed in vitro or in vivo, using natural, mutated, and/or synthetic APP substrates, natural, mutated, and/or synthetic enzyme, and the test compound. The analysis may involve primary or secondary cells expressing native, mutant, and/or synthetic APP and enzyme, animal models expressing native APP and enzyme, or may utilize transgenic animal models expressing the substrate and enzyme. Detection of enzymatic activity can be by analysis of one or more of the cleavage products, for example, by immunoassay, fluorometric or chromogenic assay, HPLC, or other means of detection. Inhibitory compounds are determined as those having the ability to decrease the amount of beta-secretase cleavage product produced in comparison to a control, where beta-secretase mediated cleavage in the reaction system is observed and measured in the absence of inhibitory compounds.

Beta-Secretase

Various forms of beta-secretase enzyme are known, and are available and useful for assay of enzyme activity and inhibition of enzyme activity. These include native, recombinant, and synthetic forms of the enzyme. Human beta-secretase is known as Beta Site APP Cleaving Enzyme (BACE), Asp2, and memapsin 2, and has been characterized, for example, in U.S. Pat. No. 5,744,346 and published PCT patent applications WO98/22597, WO00/03819, WO01/23533, and WO00/17369, as well as in literature publications (Hussain et al., 1999, Mol. Cell. Neurosci. 14:419-427; Vassar et al., 1999, Science 286:735-741; Yan et al., 1999, Nature 402:533-537; Sinha et al., 1999, Nature 40:537-540; and Lin et al., 2000, PNAS USA 97:1456-1460). Synthetic forms of the enzyme have also been described (WO98/22597 and WO00/17369). Beta-secretase can be extracted and purified from human brain tissue and can be produced in cells, for example mammalian cells expressing recombinant enzyme.

Preferred methods employ compounds that are effective to inhibit 50% of beta-secretase enzymatic activity at a concentration of less than about 50 micromolar, preferably at a concentration of less than about 10 micromolar, more preferably less than about 1 micromolar, and most preferably less than about 10 nanomolar.

APP Substrate

Assays that demonstrate inhibition of beta-secretase-mediated cleavage of APP can utilize any of the known forms of APP, including the 695 amino acid “normal” isotype described by Kang et al., 1987, Nature 325:733-6, the 770 amino acid isotype described by Kitaguchi et. al., 1981, Nature 331:530-532, and variants such as the Swedish Mutation (KM670-1NL) (APP-SW), the London Mutation (V7176F), and others. See, for example, U.S. Pat. No. 5,766,846 and also Hardy, 1992, Nature Genet. 1:233-234, for a review of known variant mutations. Additional useful substrates include the dibasic amino acid modification, APP-KK disclosed, for example, in WO 00/17369, fragments of APP, and synthetic peptides containing the beta-secretase cleavage site, wild type (WT) or mutated form, e.g., SW, as described, for example, in U.S. Pat. No. 5,942,400 and WO00/03819.

The APP substrate contains the beta-secretase cleavage site of APP (KM-DA or NL-DA) for example, a complete APP peptide or variant, an APP fragment, a recombinant or synthetic APP, or a fusion peptide. Preferably, the fusion peptide includes the beta-secretase cleavage site fused to a peptide having a moiety useful for enzymatic assay, for example, having isolation and/or detection properties. A useful moiety may be an antigenic epitope for antibody binding, a label or other detection moiety, a binding substrate, and the like.

Antibodies

Products characteristic of APP cleavage can be measured by immunoassay using various antibodies, as described, for example, in Pirttila et al., 1999, Neuro. Lett. 249:21-4, and in U.S. Pat. No. 5,612,486. Useful antibodies to detect A beta include, for example, the monoclonal antibody 6E10 (Senetek, St. Louis, Mo.) that specifically recognizes an epitope on amino acids 1-16 of the A beta peptide; antibodies 162 and 164 (New York State Institute for Basic Research, Staten Island, N.Y.) that are specific for human A beta 1-40 and 1-42, respectively; and antibodies that recognize the junction region of beta-amyloid peptide, the site between residues 16 and 17, as described in U.S. Pat. No. 5,593,846. Antibodies raised against a synthetic peptide of residues 591 to 596 of APP and SW192 antibody raised against 590-596 of the Swedish mutation are also useful in immunoassay of APP and its cleavage products, as described in U.S. Pat. Nos. 5,604,102 and 5,721,130.

Assay Systems

Assays for determining APP cleavage at the beta-secretase cleavage site are well known in the art. Exemplary assays, are described, for example, in U.S. Pat. Nos. 5,744,346 and 5,942,400, and described in the Examples below.

Cell Free Assays

Exemplary assays that can be used to demonstrate the inhibitory activity of the compounds of the invention are described, for example, in WO00/17369, WO 00/03819, and U.S. Pat. Nos. 5,942,400 and 5,744,346. Such assays can be performed in cell-free incubations or in cellular incubations using cells expressing a beta-secretase and an APP substrate having a beta-secretase cleavage site.

An APP substrate containing the beta-secretase cleavage-site of APP, for example, a complete APP or variant, an APP fragment, or a recombinant or synthetic APP substrate containing the amino acid sequence: KM-DA or NL-DA, is incubated in the presence of beta-secretase enzyme, a fragment thereof, or a synthetic or recombinant polypeptide variant having beta-secretase activity and effective to cleave the beta-secretase cleavage site of APP, under incubation conditions suitable for the cleavage activity of the enzyme. Suitable substrates optionally include derivatives that may be fusion proteins or peptides that contain the substrate peptide and a modification useful to facilitate the purification or detection of the peptide or its beta-secretase cleavage products. Useful modifications include the insertion of a known antigenic epitope for antibody binding; the linking of a label or detectable moiety, the linking of a binding substrate, and the like.

Suitable incubation conditions for a cell-free in vitro assay include, for example: approximately 200 nanomolar to 10 micromolar substrate, approximately 10 to 200 picomolar enzyme, and approximately 0.1 nanomolar to 10 micromolar inhibitor compound, in aqueous solution, at an approximate pH of 4 −7, at approximately 37 degrees C., for a time period of approximately 10 minutes to 3 hours. These incubation conditions are exemplary only, and can be varied as required for the particular assay components and/or desired measurement system. Optimization of the incubation conditions for the particular assay components should account for the specific beta-secretase enzyme used and its pH optimum, any additional enzymes and/or markers that might be used in the assay, and the like. Such optimization is routine and will not require undue experimentation.

One useful assay utilizes a fusion peptide having maltose binding protein (MBP) fused to the C-terminal 125 amino acids of APP-SW. The MBP portion is captured on an assay substrate by anti-MBP capture antibody. Incubation of the captured fusion protein in the presence of beta-secretase results in cleavage of the substrate at the beta-secretase cleavage site. Analysis of the cleavage activity can be, for example, by immunoassay of cleavage products. One such immunoassay detects a unique epitope exposed at the carboxy terminus of the cleaved fusion protein, for example, using the antibody SW192. This assay is described, for example, in U.S. Pat. No. 5,942,400.

Cellular Assay

Numerous cell-based assays can be used to analyze beta-secretase activity and/or processing of APP to release A beta. Contact of an APP substrate with a beta-secretase enzyme within the cell and in the presence or absence of a compound inhibitor of the invention can be used to demonstrate beta-secretase inhibitory activity of the compound. Preferably, assay in the presence of a useful inhibitory compound provides at least about 30%, most preferably at least about 50% inhibition of the enzymatic activity, as compared with a non-inhibited control.

In one embodiment, cells that naturally express beta-secretase are used. Alternatively, cells are modified to express a recombinant beta-secretase or synthetic variant enzyme as discussed above. The APP substrate may be added to the culture medium and is preferably expressed in the cells. Cells that naturally express APP, variant or mutant forms of APP, or cells transformed to express an isoform of APP, mutant or variant APP, recombinant or synthetic APP, APP fragment, or synthetic APP peptide or fusion protein containing the beta-secretase APP cleavage site can be used, provided that the expressed APP is permitted to contact the enzyme and enzymatic cleavage activity can be analyzed.

Human cell lines that normally process A beta from APP provide a useful means to assay inhibitory activities of the compounds of the invention. Production and release of A beta and/or other cleavage products into the culture medium can be measured, for example by immunoassay, such as Western blot or enzyme-linked immunoassay (EIA) such as by ELISA.

Cells expressing an APP substrate and an active beta-secretase can be incubated in the presence of a compound inhibitor to demonstrate inhibition of enzymatic activity as compared with a control. Activity of beta-secretase can be measured by analysis of one or more cleavage products of the APP substrate. For example, inhibition of beta-secretase activity against the substrate APP would be expected to decrease release of specific beta-secretase induced APP cleavage products such as A beta.

Although both neural and non-neural cells process and release A beta, levels of endogenous beta-secretase activity are low and often difficult to detect by EIA. The use of cell types known to have enhanced beta-secretase activity, enhanced processing of APP to A beta, and/or enhanced production of A beta are therefore preferred. For example, transfection of cells with the Swedish Mutant form of APP (APP-SW); with APP-KK; or with APP-SW-KK provides cells having enhanced beta-secretase activity and producing amounts of A beta that can be readily measured.

In such assays, for example, the cells expressing APP and beta-secretase are incubated in a culture medium under conditions suitable for beta-secretase enzymatic activity at its cleavage site on the APP substrate. On exposure of the cells to the compound inhibitor, the amount of A beta released into the medium and/or the amount of CTF99 fragments of APP in the cell lysates is reduced as compared with the control. The cleavage products of APP can be analyzed, for example, by immune reactions with specific antibodies, as discussed above.

Preferred cells for analysis of beta-secretase activity include primary human neuronal cells, primary transgenic animal neuronal cells where the transgene is APP, and other cells such as those of a stable 293 cell line expressing APP, for example, APP-SW.

In Vivo Assays: Animal Models

Various animal models can be used to analyze beta-secretase activity and/or processing of APP to release A beta, as described above. For example, transgenic animals expressing APP substrate and beta-secretase enzyme can be used to demonstrate inhibitory activity of the compounds of the invention. Certain transgenic animal models have been described, for example, in U.S. Pat. Nos. 5,877,399; 5,612,486; 5,387,742; 5,720,936; 5,850,003; 5,877,015,, and 5,811,633, and in Ganes et al., 1995, Nature 373:523. Preferred are animals that exhibit characteristics associated with the pathophysiology of AD. Administration of the compound inhibitors of the invention to the transgenic mice described herein provides an alternative method for demonstrating the inhibitory activity of the compounds. Administration of the compounds in a pharmaceutically effective carrier and via an administrative route that reaches the target tissue in an appropriate therapeutic amount is also preferred.

Inhibition of beta-secretase mediated cleavage of APP at the beta-secretase cleavage site and of A beta release can be analyzed in these animals by measure of cleavage fragments in the animal's body fluids such as cerebral fluid or tissues. Analysis of brain tissues for A beta deposits or plaques is preferred.

On contacting an APP substrate with a beta-secretase enzyme in the presence of an inhibitory compound of the invention and under conditions sufficient to permit enzymatic mediated cleavage of APP and/or release of A beta from the substrate, the compounds of the invention are effective to reduce beta-secretase-mediated cleavage of APP at the beta-secretase cleavage site and/or effective to reduce released amounts of A beta. Where such contacting is the administration of the inhibitory compounds of the invention to an animal model, for example, as described above, the compounds are effective to reduce A beta deposition in brain tissues of the animal, and to reduce the number and/or size of beta amyloid plaques. Where such administration is to a human subject, the compounds are effective to inhibit or slow the progression of disease characterized by enhanced amounts of A beta, to slow the progression of AD in the, and/or to prevent onset or development of AD in a subject at risk for the disease.

Unless defined otherwise, all scientific and technical terms used herein have the same meaning as commonly understood by one of skill in the art to which this invention belongs. All patents and publications referred to herein are hereby incorporated by reference for all purposes.

APP, amyloid precursor protein, is defined as any APP polypeptide, including APP variants, mutations, and isoforms, for example, as disclosed in U.S. Pat. No. 5,766,846.

A beta, amyloid beta peptide, is defined as any peptide resulting from beta-secretase mediated cleavage of APP, including peptides of 39, 40, 41, 42, and 43 amino acids, and extending from the beta-secretase cleavage site to amino acids 39, 40, 41, 42, or 43.

Beta-secretase (BACE1, Asp2, Memapsin 2) is an aspartyl protease that mediates cleavage of APP at the amino-terminal edge of A beta. Human beta-secretase is described, for example, in WO00/17369.

Pharmaceutically acceptable refers to those properties and/or substances that are acceptable to the subject from a pharmacological/toxicological point of view and to the manufacturing pharmaceutical chemist from a physical/chemical point of view regarding composition, formulation, stability, subject's acceptance and bioavailability.

A therapeutically effective amount is defined as an amount effective to reduce or lessen at least one symptom of the disease being treated or to reduce or delay onset of one or more clinical markers or symptoms of the disease.

It should be noted that, as used in this specification and the appended claims, the singular forms “a,” “an,” and “the” include plural referents unless the content clearly dictates otherwise. Thus, for example, reference to a composition containing “a compound” includes a mixture of two or more compounds. It should also be noted that the term “or” is generally employed in its sense including “and/or” unless the content clearly dictates otherwise.

As noted above, depending on whether asymmetric carbon atoms are present, the compounds of the invention can be present as mixtures of isomers, especially as racemates, or in the form of pure isomers, especially optical antipodes.

Salts of compounds having salt-forming groups are especially acid addition salts, salts with bases or, where several salt-forming groups are present, can also be mixed salts or internal salts.

Salts are especially the pharmaceutically acceptable or non-toxic salts of compounds of formula I.

Such salts are formed, for example, by compounds of formula I having an acid group, for example a carboxy group or a sulfo group, and are, for example, salts thereof with suitable bases, such as non-toxic metal salts derived from metals of groups Ia, Ib, IIa and IIb of the Periodic Table of the Elements, for example alkali metal salts, especially lithium, sodium or potassium salts, or alkaline earth metal salts, for example magnesium or calcium salts, also zinc salts or ammonium salts, as well as salts formed with organic amines, such as unsubstituted or hydroxy-substituted mono-, di- or tri-alkylamines, especially mono-, di- or tri-lower alkylamines, or with quaternary ammonium bases, for example with methyl-, ethyl-, diethyl- or triethyl-amine, mono-, bis- or tris-(2-hydroxy-lower alkyl)-amines, such as ethanol-, diethanol- or triethanolamine, tris(hydroxymethyl)methylamine or 2-hydroxy-tertbutylamine, N,N-di-lower alkyl-N-(hydroxy-lower alkyl)-amines, such as N,N-dimethyl-N-(2-hydroxyethyl)-amine, or N-methyl-D-glucamine, or quaternary ammonium hydroxides, such as tetrabutylammonium hydroxide. The compounds of formula I having a basic group, for example an amino group, can form acid addition salts, for example with suitable inorganic acids, for example hydrohalic acids, such as hydrochloric acid or hydrobromic acid, or sulfuric acid with replacement of one or both protons, phosphoric acid with replacement of one or more protons, e.g. orthophosphoric acid or metaphosphoric acid, or pyrophosphoric acid with replacement of one or more protons, or with organic carboxylic, sulfonic, sulfo or phosphonic acids or N-substituted sulfamic acids, for example acetic acid, propionic acid, glycolic acid, succinic acid, maleic acid, hydroxymaleic acid, methylmaleic acid, fumaric acid, malic acid, tartaric acid, gluconic acid, glucaric acid, glucuronic acid, citric acid, benzoic acid, cinnamic acid, mandelic acid, salicylic acid, 4-aminosalicylic acid, 2-phenoxybenzoic acid, 2-acetoxybenzoic acid, embonic acid, nicotinic acid or isonicotinic acid, as well as with amino acids, such as the .alpha.-amino acids mentioned hereinbefore, and with methanesulfonic acid, ethanesulfonic acid, 2-hydroxyethanesulfonic acid, ethane-1,2-disulfonic acid, benzenesulfonic acid, 4-methylbenzenenesulfonic acid, naphthalene-2-sulfonic acid, 2- or 3-phosphoglycerate, glucose-6-phosphate, or N-cyclohexylsulfamic acid (forming cyclamates) or with other acidic organic compounds, such as ascorbic acid. Compounds of formula I having acid and basic groups can also form internal salts.

For isolation and purification purposes it is also possible to use pharmaceutically unacceptable salts.

The present invention may be better understood with reference to the following examples. These examples are intended to be representative of specific embodiments of the invention, and are not intended as limiting the scope of the invention.

### BIOLOGY EXAMPLES

### Example A

Enzyme Inhibition Assay

The compounds of the invention are analyzed for inhibitory activity by use of the MBP-C125 assay. This assay determines the relative inhibition of beta-secretase cleavage of a model APP substrate, MBP-C125SW, by the compounds assayed as compared with an untreated control. A detailed description of the assay parameters can be found, for example, in U.S. Pat. No. 5,942,400. Briefly, the substrate is a fusion peptide formed of maltose binding protein (MBP) and the carboxy terminal 125 amino acids of APP-SW, the Swedish mutation. The beta-secretase enzyme is derived from human brain tissue as described in Sinha et al, 1999, Nature 40:537-540) or recombinantly produced as the full-length enzyme (amino acids 1-501), and can be prepared, for example, from 293 cells expressing the recombinant cDNA, as described in WO00/47618.

Inhibition of the enzyme is analyzed, for example, by immunoassay of the enzyme's cleavage products. One exemplary ELISA uses an anti-MBP capture antibody that is deposited on precoated and blocked 96-well high binding plates, followed by incubation with diluted enzyme reaction supernatant, incubation with a specific reporter antibody, for example, biotinylated anti-SW192 reporter antibody, and further incubation with streptavidin/alkaline phosphatase. In the assay, cleavage of the intact MBP-C125SW fusion protein results in the generation of a truncated amino-terminal fragment, exposing a new SW-192 antibody-positive epitope at the carboxy terminus. Detection is effected by a fluorescent substrate signal on cleavage by the phosphatase. ELISA only detects cleavage following Leu 596 at the substrate's APP-SW 751 mutation site.

Specific Assay Procedure:

Compounds are diluted in a 1:1 dilution series to a six-point concentration curve (two wells per concentration) in one 96-plate row per compound tested. Each of the test compounds is prepared in DMSO to make up a 10 millimolar stock solution. The stock solution is serially diluted in DMSO to obtain a final compound concentration of 200 micromolar at the high point of a 6-point dilution curve. Ten (10) microliters of each dilution is added to each of two wells on row C of a corresponding V-bottom plate to which 190 microliters of 52 millimolar NaOAc, 7.9% DMSO, pH 4.5 are pre-added. The NaOAc diluted compound plate is spun down to pellet precipitant and 20 microliters/well is transferred to a corresponding flat-bottom plate to which 30 microliters of ice-cold enzyme-substrate mixture (2.5 microliters MBP-C125SW substrate, 0.03 microliters enzyme and 24.5 microliters ice cold 0.09% TX100 per 30 microliters) is added. The final reaction mixture of 200 micromolar compound at the highest curve point is in 5% DMSO, 20 millimolar NaOAc, 0.06% TX100, at pH 4.5.

Warming the plates to 37 degrees C. starts the enzyme reaction. After 90 minutes at 37 degrees C., 200 microliters/well cold specimen diluent is added to stop the reaction and 20 microliters/well was transferred to a corresponding anti-MBP antibody coated ELISA plate for capture, containing 80 microliters/well specimen diluent. This reaction is incubated overnight at 4 degrees C. and the ELISA is developed the next day after a 2 hour incubation with anti-192SW antibody, followed by Streptavidin-AP conjugate and fluorescent substrate. The signal is read on a fluorescent plate reader.

Relative compound inhibition potency is determined by calculating the concentration of compound that showed a fifty percent reduction in detected signal (IC₅₀) compared to the enzyme reaction signal in the control wells with no added compound.

### Example B

Cell Free Inhibition Assay Utilizing a Synthetic APP Substrate

A synthetic APP substrate that can be cleaved by beta-secretase and having N-terminal biotin and made fluorescent by the covalent attachment of Oregon green at the Cys residue is used to assay beta-secretase activity in the presence or absence of the inhibitory compounds of the invention. Useful substrates include the following:

| | |
|---|---|
| Biotin-SEVNLDAEFRC[Oregon green]KK | [SEQ ID NO: 1] |
| Biotin-SEVKMDAEFRC[Oregon green]KK | [SEQ ID NO: 2] |
| Biotin-GLNIKTEEISEISYEVEFRC[Oregon green]KK | [SEQ ID NO: 3] |
| Biotin-ADRGLTTRPGSGLTNIKTEEISEVNLDAEFC[Oregon green]KK | [SEQ ID NO: 4] |
| Biotin-FVNQHLCₒₓGSHLVEALY-LVCₒₓGERGFFYTPKAC[Oregon green]KK | [SEQ ID NO: 5] |

The enzyme (0.1 nanomolar) and test compounds (0.001-100 micromolar) are incubated in pre-blocked, low affinity, black plates (384 well) at 37 degrees for 30 minutes. The reaction is initiated by addition of 150 millimolar substrate to a final volume of 30 microliter per well. The final assay conditions are: 0.001-100 micromolar compound inhibitor; 0.1 molar sodium acetate (pH 4.5); 150 nanomolar substrate; 0.1 nanomolar soluble beta-secretase; 0.001% Tween 20, and 2% DMSO. The assay mixture is incubated for 3 hours at 37 degrees C., and the reaction is terminated by the addition of a saturating concentration of immunopure streptavidin. After incubation with streptavidin at room temperature for 15 minutes, fluorescence polarization is measured, for example, using a LJL Acqurest (Ex485 nm/ Em530 nm). The activity of the beta-secretase enzyme is detected by changes in the fluorescence polarization that occur when the substrate is cleaved by the enzyme. Incubation in the presence or absence of compound inhibitor demonstrates specific inhibition of beta-secretase enzymatic cleavage of its synthetic APP substrate.

### Example C

Beta-Secretase Inhibition: P26-P4′SW Assay

Synthetic substrates containing the beta-secretase cleavage site of APP are used to assay beta-secretase activity, using the methods described, for example, in published PCT application WO00/47618. The P26-P4′SW substrate is a peptide of the sequence:

[SEQ ID NO: 6]

(biotin)CGGADRGLTTRPGSGLTNIKTEEISEVNLDAEF

The P26-P1 standard has the sequence:

[SEQ ID NO: 7]

(biotin)CGGADRGLTTRPGSGLTNIKTEEISEVNL.

Briefly, the biotin-coupled synthetic substrates are incubated at a concentration of from about 0 to about 200 micromolar in this assay. When testing inhibitory compounds, a substrate concentration of about 1.0 micromolar is preferred. Test compounds diluted in DMSO are added to the reaction mixture, with a final DMSO concentration of 5%. Controls also contain a final DMSO concentration of 5%. The concentration of beta-secretase enzyme in the reaction is varied, to give product concentrations with the linear range of the ELISA assay, about 125 to 2000 picomolar, after dilution.

The reaction mixture also includes 20 millimolar sodium acetate, pH 4.5, 0.06% Triton X100, and is incubated at 37 degrees C. for about 1 to 3 hours. Samples are then diluted in assay buffer (for example, 145.4 nanomolar sodium chloride, 9.51 millimolar sodium phosphate, 7.7 millimolar sodium azide, 0.05% Triton X405, 6 g/liter bovine serum albumin, pH 7.4) to quench the reaction, then diluted further for immunoassay of the cleavage products.

Cleavage products can be assayed by ELISA. Diluted samples and standards are incubated in assay plates coated with capture antibody, for example, SW192, for about 24 hours at 4 degrees C. After washing in TTBS buffer (150 millimolar sodium chloride, 25 millimolar Tris, 0.05% Tween 20, pH 7.5), the samples are incubated with streptavidin-AP according to the manufacturer's instructions. After a one hour incubation at room temperature, the samples are washed in TTBS and incubated with fluorescent substrate solution A (31.2 g/liter 2-amino-2-methyl-1-propanol, 30 mg/liter, pH 9.5). Reaction with streptavidin-alkaline phosphate permits detection by fluorescence. Compounds that are effective inhibitors of beta-secretase activity demonstrate reduced cleavage of the substrate as compared to a control.

### Example D

Assays using Synthetic Oligopeptide-Substrates

Synthetic oligopeptides are prepared that incorporate the known cleavage site of beta-secretase, and optionally detectable tags, such as fluorescent or chromogenic moieties. Examples of such peptides, as well as their production and detection methods are described in U.S. Pat. No. 5,942,400, herein incorporated by reference. Cleavage products can be detected using high performance liquid chromatography, or fluorescent or chromogenic detection methods appropriate to the peptide to be detected, according to methods well known in the art.

By way of example, one such peptide has the sequence (biotin)-SEVNLDAEF [SEQ ID NO: 8], and the cleavage site is between residues 5 and 6. Another preferred substrate has the sequence ADRGLTTRPGSGLTNIKTEEISEVNLDAEF [SEQ ID NO: 9], and the cleavage site is between residues 26 and 27.

These synthetic APP substrates are incubated in the presence of beta-secretase under conditions sufficient to result in beta-secretase mediated cleavage of the substrate. Comparison of the cleavage results in the presence of the compound inhibitor to control results provides a measure of the compound's inhibitory activity.

### Example E

Inhibition of Beta-Secretase Activity—Cellular Assay

An exemplary assay for the analysis of inhibition of beta-secretase activity utilizes the human embryonic kidney cell line HEKp293 (ATCC Accession No. CRL-1573) transfected with APP751 containing the naturally occurring double mutation Lys651Met52 to Asn651Leu652 (numbered for APP751), commonly called the Swedish mutation and shown to overproduce A beta (Citron et al., 1992, Nature 360:672-674), as described in U.S. Pat. No. 5,604,102.

The cells are incubated in the presence/absence of the inhibitory compound (diluted in DMSO) at the desired concentration, generally up to 10 micrograms/ml. At the end of the treatment period, conditioned media is analyzed for beta-secretase activity, for example, by analysis of cleavage fragments. A beta can be analyzed by immunoassay, using specific detection antibodies. The enzymatic activity is measured in the presence and absence of the compound inhibitors to demonstrate specific inhibition of beta-secretase mediated cleavage of APP substrate.

### Example F

Inhibition of Beta-Secretase in Animal Models of AD

Various animal models can be used to screen for inhibition of beta-secretase activity. Examples of animal models useful in the invention include, but are not limited to, mouse, guinea pig, dog, and the like. The animals used can be wild type, transgenic, or knockout models. In addition, mammalian models can express mutations in APP, such as APP695-SW and the like described herein. Examples of transgenic non-human mammalian models are described in U.S. Pat. Nos. 5,604,102, 5,912,410 and 5,811,633.

PDAPP mice, prepared as described in Games et al., 1995, Nature 373:523-527 are useful to analyze in vivo suppression of A beta release in the presence of putative inhibitory compounds. As described in U.S. Pat. No. 6,191,166, 4 month old PDAPP mice are administered compound formulated in vehicle, such as corn oil. The mice are dosed with compound (1-30 mg/ml; preferably 1-10 mg/ml). After time, e.g., 3-10 hours, the animals are sacrificed, and brains removed for analysis.

Transgenic animals are administered an amount of the compound inhibitor formulated in a carrier suitable for the chosen mode of administration. Control animals are untreated, treated with vehicle, or treated with an inactive compound. Administration can be acute, i.e., single dose or multiple doses in one day, or can be chronic, i.e., dosing is repeated daily for a period of days. Beginning at time 0, brain tissue or cerebral fluid is obtained from selected animals and analyzed for the presence of APP cleavage peptides, including A beta, for example, by immunoassay using specific antibodies for A beta detection. At the end of the test period, animals are sacrificed and brain tissue or cerebral fluid is analyzed for the presence of A beta and/or beta-amyloid plaques. The tissue is also analyzed for necrosis.

Animals administered the compound inhibitors of the invention are expected to demonstrate reduced A beta in brain tissues or cerebral fluids and reduced beta amyloid plaques in brain tissue, as compared with non-treated controls.

### Example G

Inhibition of A Beta Production in Human Subjects

Subjects suffering from Alzheimer's Disease (AD) demonstrate an increased amount of A beta in the brain. AD subjects and patients are administered an amount of the compound inhibitor formulated in a carrier suitable for the chosen mode of administration. Administration is repeated daily for the duration of the test period. Beginning on day 0, cognitive and memory tests are performed, for example, once per month.

Subjects administered the compound inhibitors are expected to demonstrate slowing or stabilization of disease progression as analyzed by changes in one or more of the following disease parameters: A beta present in CSF or plasma; brain or hippocampal volume; A beta deposits in the brain; amyloid plaque in the brain; and scores for cognitive and memory function, as compared with control, non-treated subjects.

### Example H

Prevention of A Beta Production in Subjects at Risk for AD

Subjects predisposed or at risk for developing AD are identified either by recognition of a familial inheritance pattern, for example, presence of the Swedish Mutation, and/or by monitoring diagnostic parameters. Subjects identified as predisposed or at risk for developing AD are administered an amount of the compound inhibitor formulated in a carrier suitable for the chosen mode of administration. Administration is repeated daily for the duration of the test period. Beginning on day 0, cognitive and memory tests are performed, for example, once per month.

Subjects administered the compound inhibitors are expected to demonstrate slowing or stabilization of disease progression as analyzed by changes in one or more of the following disease parameters: A beta present in CSF or plasma; brain or hippocampal volume; amyloid plaque in the brain; and scores for cognitive and memory function, as compared with control, non-treated subjects.

Preparation of the Compounds

Schemes I-III for preparing the compounds employed in the methods of this invention are presented below. Tables I and II, which follow the schemes, illustrate the compounds that can be synthesized by Schemes I-III, but Schemes I-III are not limited by the compounds in the tables nor by any particular substituents employed in the schemes for illustrative purposes. The examples specifically illustrate the application of the following schemes to specific compounds.

Amide couplings used to form the compounds of this invention are typically performed by the carbodiimide method with reagents such as dicyclohexylcarbodiimide, or 1-ethyl-3-(3-dimethylaminopropyl) carbodiimide. Other methods of forming the amide or peptide bond include, but are not limited to the synthetic routes via an acid chloride, azide, mixed anhydride or activated ester. Typically, solution phase amide coupling are performed, but solid-phase synthesis by classical Merrifield techniques may be employed instead. The addition and removal of one or more protecting groups is also typical practice.

Additional related information on synthetic background is contained in EPO 0337714.

One method for producing formula I compounds is provided by Scheme I. Dihydro-5(S)-(tert-butyldimethylsilyloxymethyl)-3(2H)-furanone (compound 1 below) is prepared by standard methods known in the art from commercially available dihydro-5(S)-(hydroxy-methyl)-2(3H)-furanone. After alkylation of compound 1 to form compound 2, the protecting group of lactone 2 is removed with aqueous HF to afford compound 3.

The alcohol group of 3 is activated by conversion into a leaving group such as mesylate, tosylate or trifylate by treating the alcohol with a sulfonyl chloride or sulfonic anhydride, such as trifluoromethanesulfonic anhydride, in the presence of a hindered amine base such as triethylamine, diethyl isopropylamine or 2,6 lutidine, to afford a compound such as compound 4. The leaving group of compound 4 is displaced by an amine 5, such as N′-t-butyl-(4aS,8aS)-(decahydroisoquinoline)-3(S)-carboxamide, in a high boiling solvent such as DMF or xylene to produce a compound such as 6. A trifluoromethanesulfonyloxy group can be displaced by an amine at room temperature in a solvent such as isopropanol by treatment with N,N-diisopropylethylamine.

Compound 6 is hydrolyzed with aqueous lithium or sodium hydroxide and the resultant hydroxy acid 7 is converted into a protected hydroxy acid 8. The hydroxyl group is conveniently protected with a standard silyl protecting group such as t-butyldimethyl silyl or t-butyldiphenyl silyl.

The protected hydroxy-acid 8 is then coupled to the desired R¹² amine to produce compound 9, and the silyl protecting group is removed with fluoride ion to arrive at compound 10.

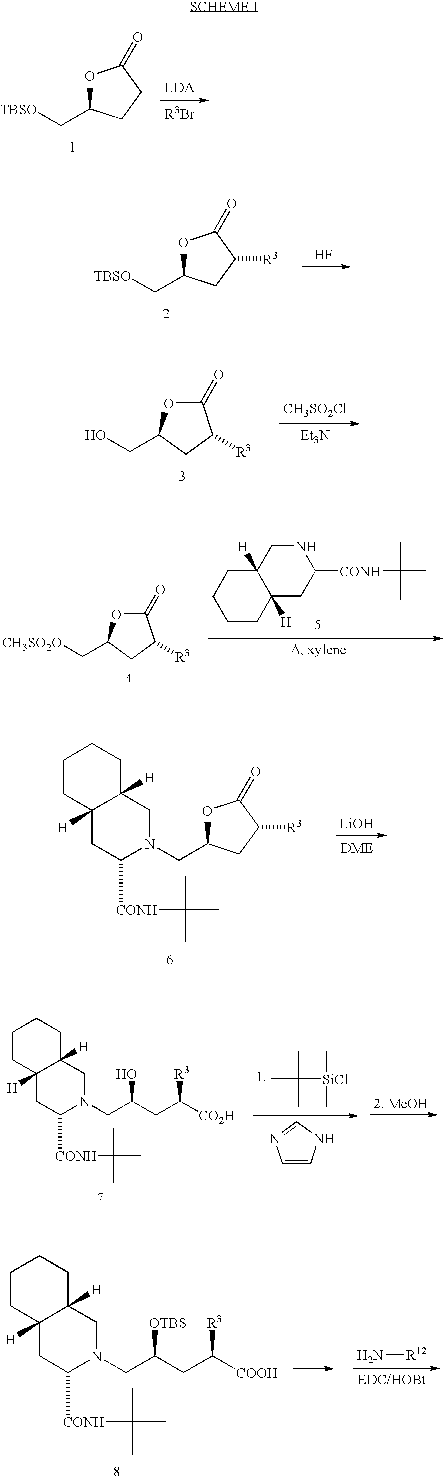

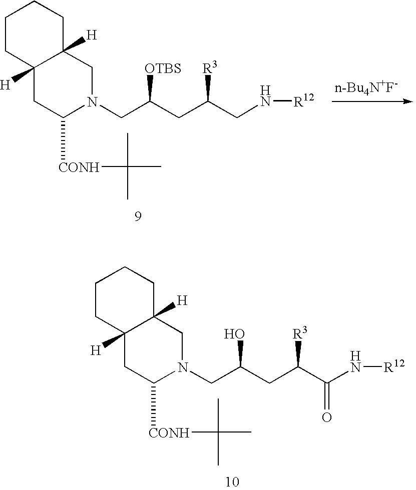

A second method for forming products of general formula I is shown in Scheme II. In Scheme II, alkylation of 11 is performed by a first step of deprotonation of 11 with n-butyllithium or lithium diisopropylamide (LDA) followed by a second step of adding an alkenyl halide (such as allyl bromide) to, afford 12.

Dihydroxylation of the olefin of 12 with osmium tetroxide and N-methylmorpholine-N-oxide (NMO) produces a diasteriomeric mixture of diols, 13. Selective mesylation of the primary alcohol of 13 with methanesulfonyl chloride and either triethylamine or pyridine gives a mesylate 14.

Heating mesylate 14 with an amine in a refluxing alcoholic solvent such as methanol or isopropanol which contains an excess of potassium carbonate produces an amino alcohol such as compound 15. The diasteriomers can be separated at this step by standard techniques well known to those of skill in the art. Alternatively, the separation can be done after removal of the ketal.

Removal of the ketal in compound 15 is accomplished by treatment with acid in the presence of methanol, or by aqueous 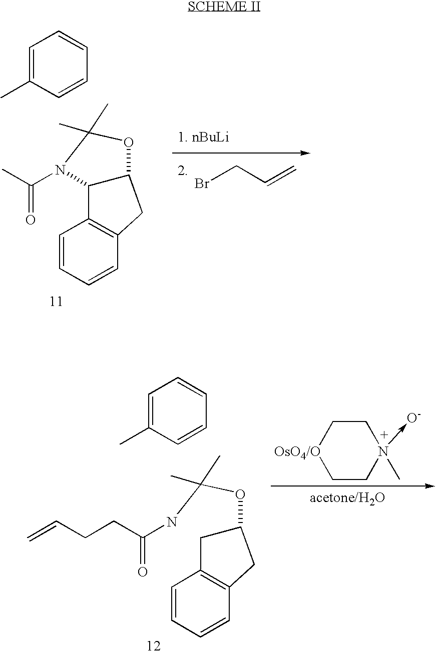
acid or by 1N HCl in THF, to form compound 16.

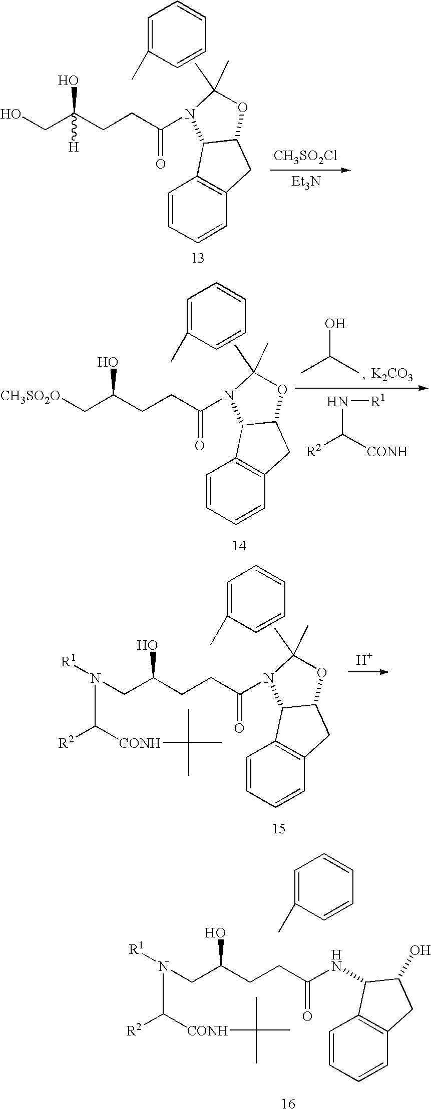

A third method for forming products of general formula I is shown in Scheme III. Protection of the pyrrolidine —NH— group of compound 17 is carried out with BOC-anhydride and dimethylaminopyridine to give the protected compound 18. Alkylation of 18 is performed by a first step of deprotonation of 18 with a strong base such as lithium hexamethyldisilamide (LHMDS) or lithium diisopropylamide (LIDA) followed by a second step of adding an alkyl halide (such as benzyl bromide) to afford compound 19.

The TBS protecting and BOC protecting group of 19 are removed by treatment with aqueous HF in acetonitrile to give alcohol 20. Mesylation of the primary alcohol of 20 with methanesulfonyl chloride and either triethylamine or pyridine gives mesylate 21 which is heated with an amine in a refluxing alcoholic solvent such as methanol or isopropanol which contains an excess of potassium carbonate to produce an amino pyrrolidinone such as compound 22. The pyrrolidine —NH— group of 22 is reprotected as a BOC group as before and the resultant compound 23 is hydrolized open with a base such as lithium or sodium hydroxide to afford the acid 24. Compound 24 is then coupled to an NH₂R¹² amine in a standard manner and the BOC is removed with gaseous HCl or trifluoroacetic acid to give the desired product, exemplified by compound 25. 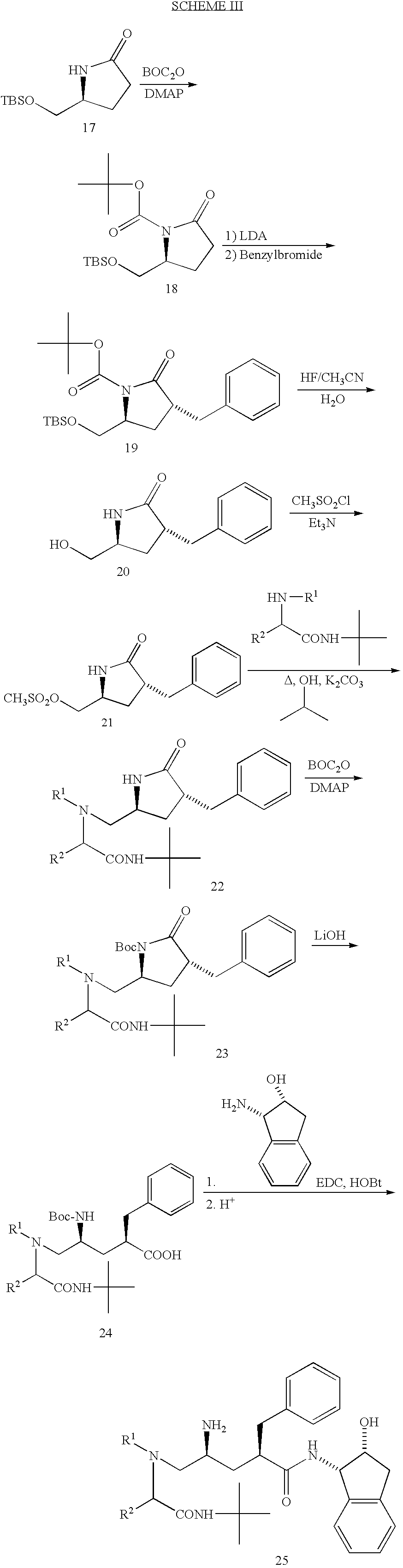
A compound of formula 26 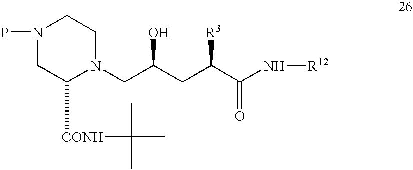
wherein P is a nitrogen protecting group such as —BOC or —CBZ, is preferably prepared according to the method described in Scheme I, preferably employing the 5-trifluoromethanesulfonyloxymethyl analog of lactone 4 therein (see Example 15, Step 1).
Compounds of formula 27 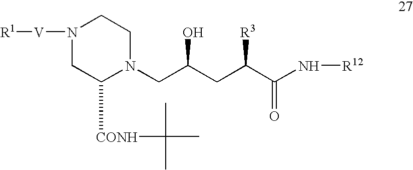
can be obtained by a variety of routes from compound 28 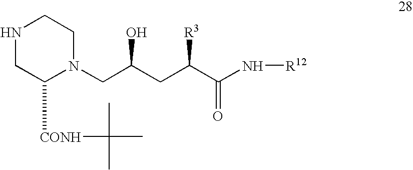
which is obtained after removal of the nitrogen protecting group in 26 using methods well known in the art, e.g., catalytic hydrogenation to remove a CBZ group, or treatment with trimethylsilyltriflate and 2,6 lutidine at about 0° C. in a solvent such as CH₂Cl₂to remove a BOC group.

For example, the 4-position piperazinyl nitrogen of compound 28 can be alkylated with a compound of formula R¹—X in a solvent such as DMF in the presence of Et₃N at room temperature, wherein X is —Cl, Br or —I, or a sulfonamide group can be formed by treatment of 28 with a sulfonyl chloride compound of formula R¹ SO₂Cl under similar conditions. Also, standard amide coupling techniques can be used to form an amide group at the piperazinyl 4-position. Techniques for these procedures are well known to those skilled in the art. The R¹ group of R¹ —X or R¹ SO₂Cl is defined above in the definition of compounds of formula I wherein R¹ is independent from and not joined to R², except that R¹ can not be hydrogen or a group with a free hydroxy substituent, such as —C₁₋₄ alkyl substitued with hydroxy, with the further exception that R¹ can be aryl substituted with a hydroxy group.

The compounds employed in the methods of this invention are also illustrated by Tables I-IV, which follow.

TABLE I

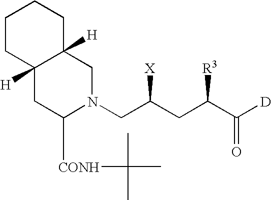

| R³ | X | D |
|---|---|---|
| —CH₂—Ph | —OH | 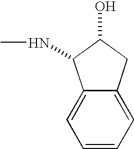 |
| —CH₂—Ph | —OH | 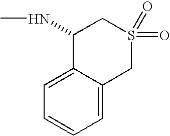 |
| —CH₂—Ph | —OH | 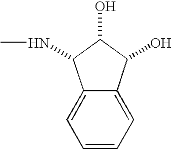 |
| —CH₂—Ph | —OH | 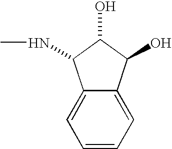 |
| —CH₂—Ph | —OH | 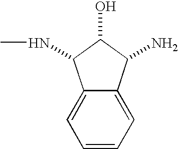 |
| —CH₂—Ph | —OH | 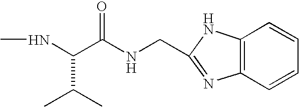 |
| —CH₂—Ph | —OH | 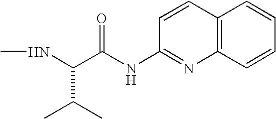 |
| —CH₂—Ph | —OH | 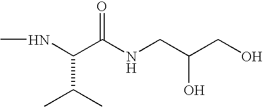 |
| —CH₂—Ph | —OH | 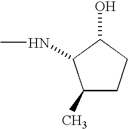 |
| —CH₂—Ph | —OH | 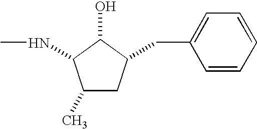 |
| —CH₂—Ph | —OH | 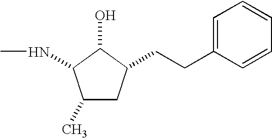 |
| —CH₂—Ph | —OH | 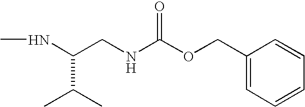 |
| —CH₂—Ph | —OH | 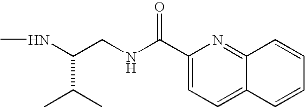 |
| —CH₂—Ph | —OH | 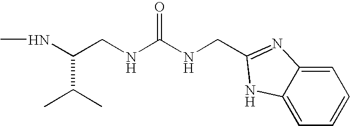 |
| —CH₂—Ph | —OH | 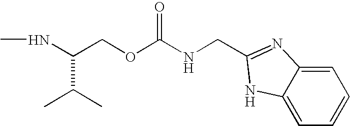 |
| —CH₂—Ph | —OH | 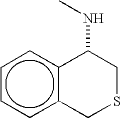 |
| —CH₂—Ph | —NH₂ | 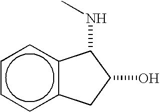 |
| —CH₂—Ph | —OH | 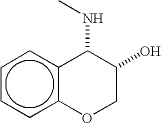 |
| —CH₂—Ph | —OH | 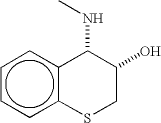 |
| —CH₂—Ph | —NH₂ | 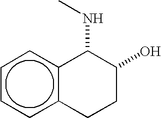 |
| —CH₂—Ph | —OH | 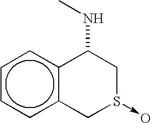 |
| —CH₂—Ph | —OH | 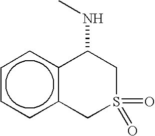 |
| —CH₂—Ph | —OH | 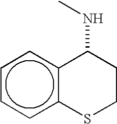 |
| 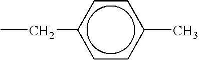 | —OH | 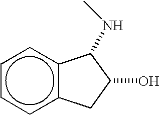 |
| 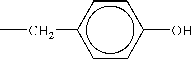 | —OH | 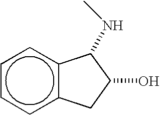 |
| 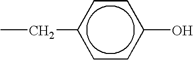 | —OH | 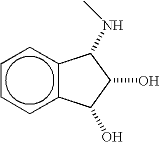 |
| 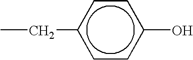 | —NH₂ | 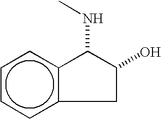 |
| 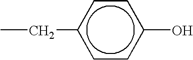 | —NH₂ | 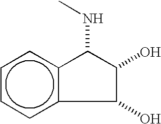 |
| 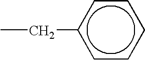 | —OH | 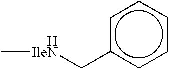 |
| 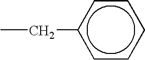 | —OH | 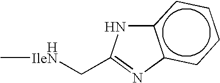 |
| 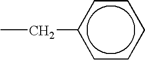 | —NH₂ | 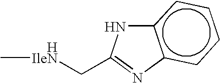 |
| 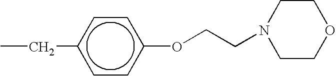 | —OH | 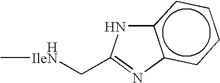 |
| 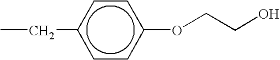 | —OH | 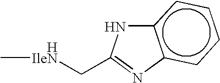 |
| 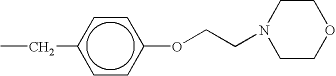 | —NH₂ | 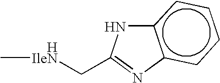 |
| 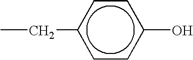 | —OH | 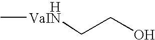 |
| 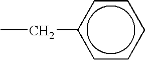 | —OH | 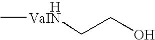 |
| 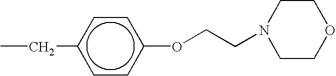 | —OH | 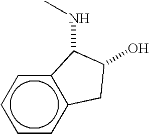 |
| 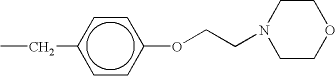 | —NH₂ | 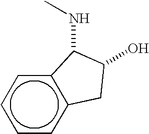 |
| 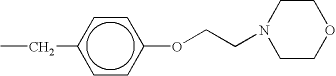 | —OH | 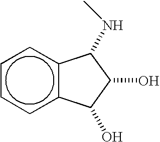 |
| 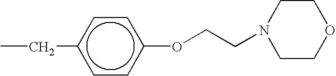 | —OH | 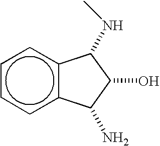 |
| 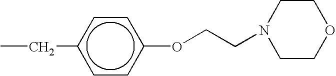 | —NH₂ | 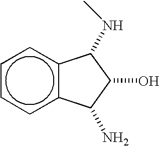 |
| 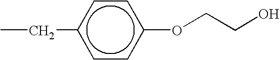 | —OH | 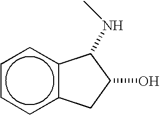 |
| 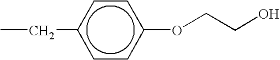 | —OH | 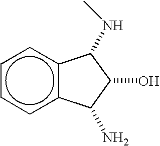 |
| 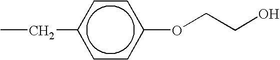 | —NH₂ | 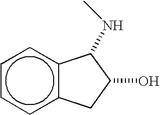 |
| 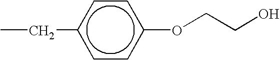 | —NH₂ | 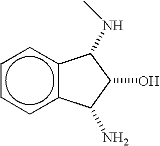 |
| 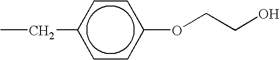 | —OH | 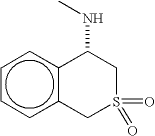 |
| —CH₂CH═CH—Ph | —OH | 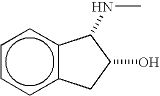 |
| —CH₂CH═CH—Ph | —OH | 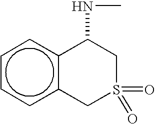 |
| 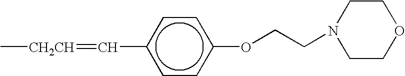 | —OH | 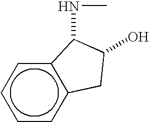 |
| 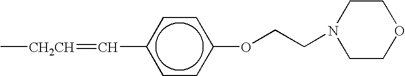 | —OH | 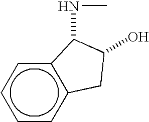 |

TABLE II

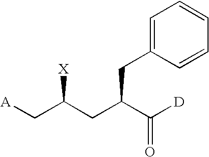

| A | X | D |
|---|---|---|
| 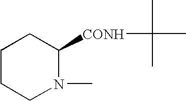 | —OH | 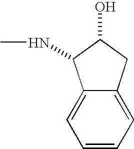 |
| 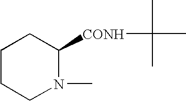 | —OH | 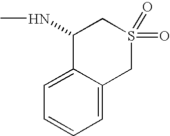 |
| 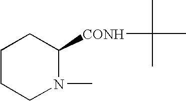 | —NH₂ | 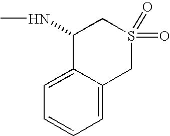 |
| 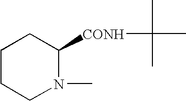 | —NH₂ | 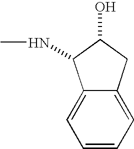 |
| 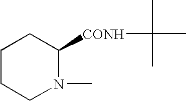 | —OH | 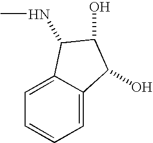 |
| 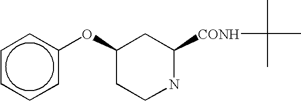 | —OH | 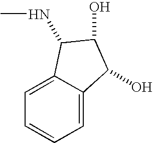 |
| 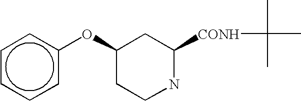 | —OH | 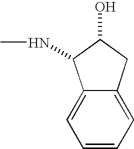 |
| 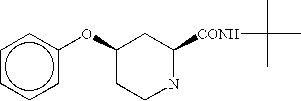 | —OH | 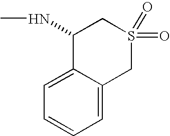 |
| 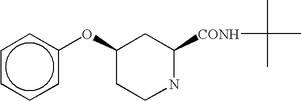 | —NH₂ | 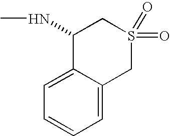 |
| 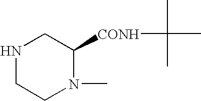 | —OH | 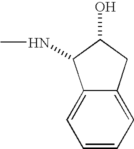 |
| 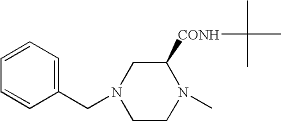 | —OH | 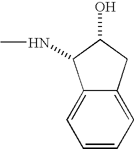 |
| 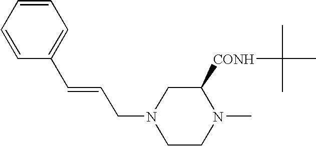 | —OH | 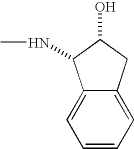 |
| 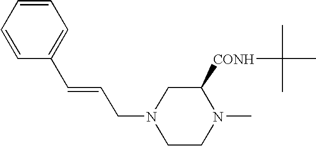 | —OH | 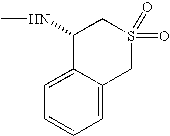 |
| 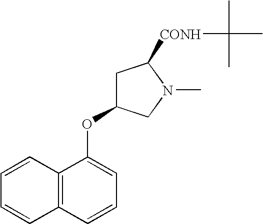 | —OH | 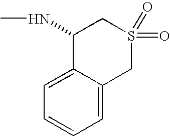 |
| 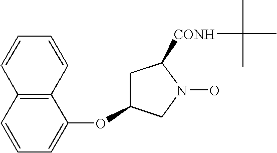 | —OH | 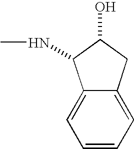 |
| 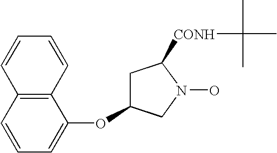 | —OH | 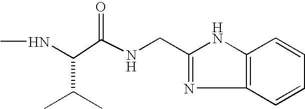 |
| 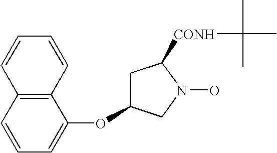 | —OH | 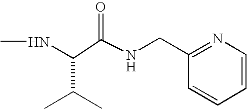 |
| 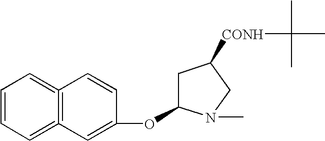 | —OH | 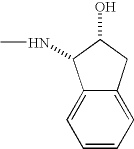 |
| 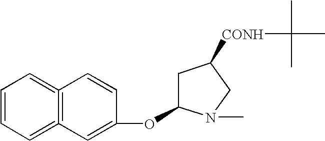 | —OH | 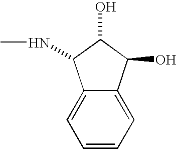 |
| 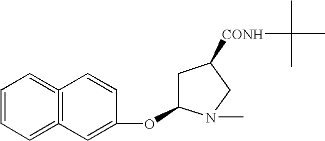 | —OH | 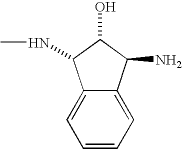 |
| 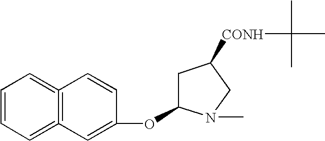 | —OH | 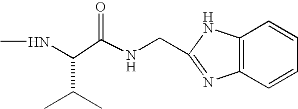 |
| 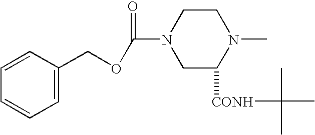 | —OH | 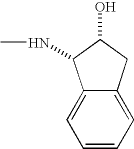 |
| 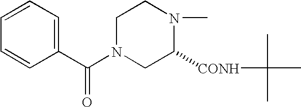 | —OH | 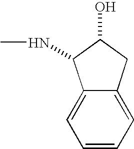 |
| 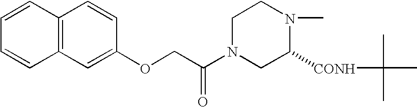 | —OH | 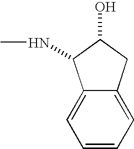 |
| 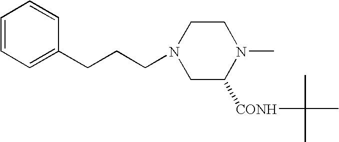 | —OH | 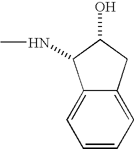 |
| 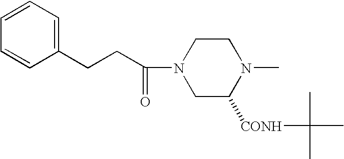 | —OH | 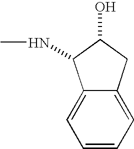 |
| 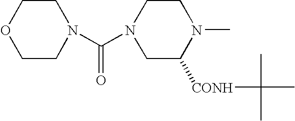 | —OH | 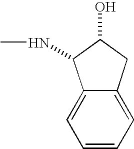 |
| 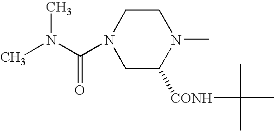 | —OH | 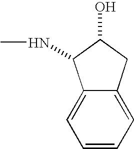 |

TABLE III

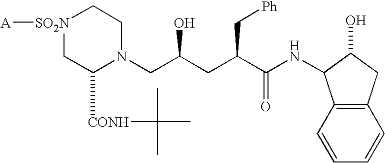

| A | A, con't |
|---|---|
| 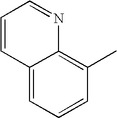 | CH₃CH₂CH₂CH₂— |
| CH₃— | (CH₃)₂CH— |
| 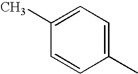 | 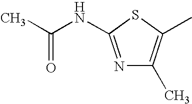 |
| 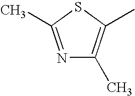 | 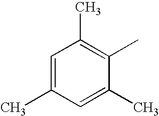 |
| 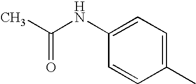 | 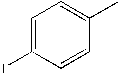 |
| 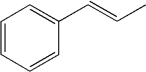 | 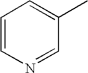 |

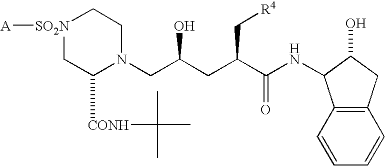

| A | R⁴ |
|---|---|
| 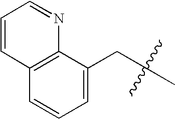 | 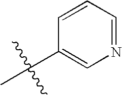 |

TABLE IV

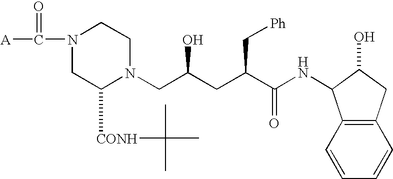

A

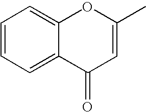

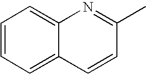

(CH₃)₃C—O—

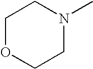

Ph—CH₂CH₂—

### Example 1

### Preparation of N-(2(R)-hydroxy-1(S)-indanyl)-2(R)-phenylmethyl-4(S)-hydroxy-5-(1-(N′-(t-butyl)-4(S)-phenoxyprolineamide)yl)-pentaneamide

### Step 1: Preparation of N-(2(R)-hydroxy-1(S)-indanyl)-3-phenylpropaneamide

To a cold (0° C.) solution of methylene chloride (30 ml) containing 2(R)-hydroxy-1(S)-aminoindane (750 mg, 5.0 mmol) and triethylamine (606 mg, 6.0 mmol) was added a solution of hydrocinnamoyl chloride (843 mg, 5.0 mmol) in 5 ml of methylene chloride. After 2 hr the reaction was poured into a separatory funnel containing 50 ml of methylene chloride and washed with 10% citric acid solution (2×30 ml). The organic layer was dried, filtered and concentrated to afford a white solid.

### Step 2: Preparation of N-(2(R)-hydroxy-1(S)-indan-N,O-isopropylidene-yl)-3-phenyl-propaneamide

The crude white solid from step 1 above was dissolved in 50 ml of methylene chloride and 5 ml of dimethoxypropane was added followed by the addition of 100 mg of p-toluenesulfonic acid. The reaction was stirred at room temperature for 18 hr and then poured into a separatory funnel and washed with saturated NaHCO₃solution (2×30 ml). The organic layer was dried, filtered and concentrated to afford an oil which was chromatographed (SiO₂, 40% EtOAc/Hexane) to give an oil which eventually crystallized.

### Step 3: Preparation of N-(2(R)-hydroxy-1(S)-indan-N,O-isopropylidene-yl)-2(S)-phenylmethyl-pent-4 -eneamide

To a solution of N-(2(R)-hydroxy-1(S)-indan-N,O-isopropylidene-yl)-3-phenyl-propaneamide (1.03 gm, 2.9 mmol) in 20 ml of THF cooled to −78° C. was added n-BuLi (2.5 M, 1.40 ml, 3.5 mmol). After 20 min, allyl bromide (0.48 gm, 3.9 mmol) was added, the reaction was stirred at −78° C. for 1 hr and then 10 ml of saturated NH₄Cl solution was added to quench the reaction. The reaction was diluted with 50 ml of water, extracted with ethyl acetate (2×50 ml), the organic phase was washed with saturated NaCl solution (50 ml), dried filtered and concentrated to afford the crude product. The crude product was purified on silica gel to afford the title compound.

### Step 4: Preparation of N-(2(R)-hydroxy-1(S)-indan-N,O-isopropylidene-yl)-2(S)-phenylmethyl-(4(RS),5-dihydroxy)-pentaneamide

To 800 mg (2.2 mmol) of N-(2(R)-hydroxy-1(S)-indan-N,O-isopropylidene-yl)-2(S)-phenylmethyl-pent-4 -en eamide dissolved in 40 ml of a 9:1 mixture of acetone/water was added 0.8 ml of a 60% solution of N-methylmorpholine-N-oxide in water followed by 4 ml of a 2.5% solution of osmium tetroxide in t-BuOH. After 18 hr, excess solid sodium bisulfate was added, the reaction was stirred for 2 hr and then filtered through a pad of celite. The filtrate was concentrated, diluted with 50 ml of water, extracted with methylene chloride (2×50 ml), the organic phase was dried, filtered and concentrated to give the product as a foam.

### Step 5: Preparation of N-(2(R)-hydroxy-1(S)-indan-N,O-isopropylidene-yl)-2(S)-phenylmethyl-4(RS)-hydroxy-5-methanesulfonyloxy-pentaneamide

To 200 mg (0.527 mmol) of N-(2(R)-hydroxy-1(S)-indan-N,O-isopropylidene-yl)-2(S)-phenylmethyl-(4(RS),5-dihydroxy)-pentaneamide dissolved in 7 ml of methylene chloride at 0° C. was added triethylamine (59 mg, 0.58 mmol), followed by methanesulfonyl chloride (66 mg, 0.579 mmol). After 4 hr the reaction was worked up by washing with 10% citric acid solution (2×50 ml) and the organic phase was dried, filtered and concentrated to afford the monomesylate as a mixture of alcohols.

### Step 6: Preparation of N′-t-butyl-N-Boc-4(R)-hydroxy-L-prolineamide

To a solution of N-Boc-4(R)-hydroxyproline (2.00 g) in DMF (20 mL) cooled to 0° C. was added EDC (1.987 g), HOBt (1.401 g), tert butyl amine (1.09 mL) and triethylamine (2.41 mL). After 18 h the reaction mixture was diluted with ethyl acetate (150 mL) and washed with 10% HCl, saturated NaHCO₃, water and brine. The solution was then dried over MgSO₄ and concentrated to afford a white solid.

### Step 7: Preparation of N,-t-butyl-N-Boc-4(S)-phenoxy-L-prolineamide

To a solution of N′-t-butyl-N-Boc-4(R)-hydroxy-L-prolineamide (0.6 g) in THF (5 mL) was added phenol (0.295 g), triphenylphosphine (0.824 g) and then diethylazo-dicarboxylate (0.495 mL) dropwise. The reaction mixture stirred for 24 h at ambient temperature and was diluted with ethyl acetate (200 mL) and washed with saturated NaHCO₃, water, brine and dried over MgSO₄. Concentration in vacuo afforded a yellow oil which was purified by flash chromatography (elution hexane: EtOAc 1:1, 30 mm column).

### Step 8: Preparation of N-t-butyl-4(S)-phenoxy-L-prolineamide trifluoroacetic acid salt

To a solution of N′-t-butyl-N-Boc-4(S)-phenoxy-L-prolineamide (0.596 g) in methylene chloride (4 mL) at 0° C. was added trifluoroacetic acid (2 mL). After 30 min the reaction was warmed to room temperature and stirred for two hours. The solvent was removed in vacuo and a slightly yellow oil was obtained.

### Step 9: Preparation of N-(2(R)-hydroxy-1(S)-indan-N,O-isopropylidene-yl)-2-(R)-phenylmethyl-4-(S)-hydroxy-5-(1-(N′-(t-butyl)-4(S)-phenoxy-prolineamide)yl)-pentaneamide

To a solution of N-t-butyl-4(S)-phenoxy-L-prolineamide trifloroacetic acid salt (0.36 g) and N-(2(R)-hydroxy-1(S)-indan-N,O-isopropylidene-yl)-2(S)-phenylmethyl-4(RS)- hydroxy-5-methanesulfonyloxy-pentaneamide (0.226 g) in 3 mL of isopropanol was added potassium carbonate (0.441 g) and the reaction was warmed to 80° C. After 18 h the reaction was cooled to room temperature, filtered through celite which was washed with further portions of EtOAc. The filtrate was concentrated, the residue was dissolved in EtOAc (100 mL) and washed with water, brine and dried over MgSO₄. The solvent was removed in vacuo and the resulting oil was purified by flash chromatography to afford the product as a mixture of diastereomers.

### Step 10: Prep of N-(2(R)-hydroxy-1S)-indanyl)-2-(R)-phenylmethyl-4-(S)-hydroxy-5-(1-(N′-t-butyl-4(S)-phenoxyprolineamid)yl)-pentaneamide

To a solution of N-(2(R)-hydroxy-1(S)-indan-N,O-isopropylidene-yl)-2-(R)-phenylmethyl-4-(S)-hydroxy-5-(1-(N′-(t-butyl)-4(S)-phenoxyprolineamide)yl)-pentaneamide (0.13 g) in MeOH (5 mL) was added camphorsulfonic acid (CSA) (0.070 g) at ambient temperature. After 5 hours more CSA (0.025 g) was added and the reaction was stirred for total of 18 hours. The reaction was quenched with saturated NaHCO₃(5 mL) and the solvent was removed to a volume of 4 mL. The aqueous layer was thoroughly extracted with EtOAc and the organic layer was washed with water, brine and dried. After removal of the solvent in vacuo the resulting oil was purified via flash chromatography to provide the title compound as a white foam. The foam was dissolved in EtOAc: hexanes and the mother liquor was decanted away from the oil. The oil was then dried in a high vacuum desiccator to afford a white foam.

### Example 2

### Preparation of N-(2(R)-hydroxy-1(S)-indanyl)-2(R)-phenylmethyl-4(S)-hydroxy-5-(1-(N′-t-butyl-4(S)-2-naphthyloxy-prolineamid)yl)-pentaneamide

### Step 1: Preparation of N-t-butyl-4(S)-2-naphthyloxy-L-prolineamide trifluoroacetic acid salt

Following substantially the same procedure for synthesizing N-t-butyl-4(S)-phenoxy-L-prolineamide trifluoroacetic acid salt as outlined in Example 1, Steps 6 through 8, but substituting 2-naphthol for the phenol used therein, the 2-naphthyloxy proline amide was produced.

### Step 2: Preparation of N-(2(R)-hydroxy-1(S)-indanyl)-2(R)-phenylmethyl-4(S)-hydroxy-5-(1-(N′-t-butyl-4(S)-2-naphthyloxy-prolineamid)yl)-pentaneamide

The title compound was produced by following substantially the same procedure outlined in Example 1, Steps 9 and 10, but substituting N-t-butyl-4(S)-2-naphthyloxy-L-prolineamide trifluoroacetic acid salt for the N-t-butyl-4(S)-phenoxy-L-prolineamide trifloroacetic acid salt used in step 9 therein.

### Example 3

### Preparation of N-(2(R)-hydroxy-1(S)-indanyl)-2(R)-phenylmethyl-4(S)-hydroxy-5-(1-(N′-t-butyl-4(S)-1-naphthyloxy-prolineamid)yl)-pentaneamide

### Step 1: Preparation of N-t-butyl-4(S)-1-naphthyloxy-L-prolineamide trifluoroacetic acid salt

Following substantially the same procedure for synthesizing N-t-butyl-4(S)-phenoxy-L-prolineamide trifluoroacetic acid salt as outlined in Example 1, Steps 6 through 8, but substituting 1-naphthol for the phenol used therein, the 1-naphthyloxy proline amide was produced.

### Step 2: Preparation of N-(2(R)-hydroxy-1(S)-indanyl)-2(R)-phenylmethyl-4(S)-hydroxy-5-(1-(N′-t-butyl-4(S)-2-naphthyloxy-prolineamid)yl)-pentaneamide

The title compound was produced by following the procedure outlined in Example 1, Steps 9 and 10, but substituting N-t-butyl-4(S)-1-naphthyloxy-L-prolineamide trifluoroacetic acid salt for the N-t-butyl-4(S)-phenoxy-L-prolineamide trifluoroacetic acid salt used in Step 9.

### Example 4

### Preparation of N-(2(R)-hydroxy-1(S)-indanyl)-2(R)-phenylmethyl-4(S)-hydroxy-5-(2-(3 (S)-N′- (t-butyl-carboxamido)-(4aS,8aS )-decahydroisoquinoline)yl)-pentaneamide

### Step 1: Preparation of dihydro-5(S)-((t-butyldiphenylsilyl)oxymethyl)-3(R)phenylmethyl-3(2H)-furanone

A solution of lithium diisopropylamide (LDA) was generated by the addition 1.55 ml of n-BuLi (2.5 M in hexane) to 0.55 ml (3.9 mmol) of diisopropylamine in 10 ml of THF at −78° C. After 30 minutes a solution of dihydro-5-(S)-((t-butyldiphenylsilyl)-oxymethyl)-3(2H)-furanone (1.38 g, 3.89 mmol) in 5 ml of THF was added. After an additional 30 minutes of stirring, benzyl bromide (0.68 g, 3.9 mmol) was added and stirring was continued for 3 h after which time the reaction was quenched with the addition of a 10% aqueous citric acid solution. The solution was extracted with ethyl acetate (2×50 ml) which was backwashed with brine, dried, filtered and concentrated to afford an oil. The product was purified by chromatography (SiO₂, 20% EtOAc/Hexane) to afford the title compound.

### Step 2: Preparation of dihydro-5(S)-(hydroxy-methyl)-3(R)-phenylmethyl-3(2H)-furanone

To 5.26 g of dihydro-5(S)-((t-butyldiphenylsilyl)oxymethyl)-3(R)phenylmethyl-3(2H)-furanone in 40 ml of acetonitrile was added 1.34 ml of a 49% aqueous HF solution. After 18 hr at room temperature the reaction was concentrated to dryness and the residue was partitioned between water (50 ml) and ethyl acetate (50 ml). The organic layer was washed with brine, dried filtered and concentrated to afford the product as a tan solid (mp 69°-72° C.).

### Step 3: Preparation of dihydro-5(S)-((methanesulfonyl)oxyymethyl)-3(R)phenylmethyl-3(2H)-furanone

To a solution of 2.93 g (14 mmol) of dihydro-5(S)-(hydroxymethyl)-3(R)-phenylmethyl-3(2H)-furanone in methylene chloride cooled to 0° C. was added triethylamine (1.98 ml, 15.6 mmol) followed by the addition of methanesulfonyl chloride (1.20 ml, 15.6 mmol). After 1 hour at 0° C., the reaction was poured into 10% aqueous citric acid solution, washed with ethyl acetate (2×100 ml) which was backwashed with water (100 ml), brine (100 ml), dried, filtered and concentrated to give the product as a waxy brown solid.

### Step 4: Preparation of dihydro-5(S)-(2-(3(S)-N-(t-butylcarboxamido)-(4aS,8aS)-(decahydroisoquinoline)yl)methyl)-3(R)-phenylmethyl-3(2H)-furanone

To 70 mg of dihydro-5(S)-((methanesulfonyl)-oxymethyl)-3(R)phenylmethyl-3(2H)-furanone (0.25 mmol) in 10 ml of xylene containing 100 mg of potassium carbonate was added 65 mg (0.27 mmol) of N-t-butyl-(4aS,8aS)-(decahydroisoquinoline)-3(S)-carboxamide and the reaction was heated to 140° C. After 6 hours, the reaction was cooled, poured into 30 ml of water which was washed with ethyl acetate (2×30 ml). The organic phase was dried, filtered and concentrated to afford a residue which was chromatographed (50/50 EtOAc/Hexane) to give the product.

### Step 5: Preparation of 2(R)-phenylmethyl-4(S)-(t-butyldimethylsilyloxy)-5-(2-(3(S)-N-(t-butylcarboxamido)-(4aS,8aS)-decahydroisoquinoline)yl)-pentanoic acid

To 130 mg (0.305 mmol) of dihydro-5(S)-(2-(3(S)-N-(t-butylcarboxamido)-(4aS,8aS)-(decahydroisoquinoline)yl)methyl)-3(R)-phenylmethyl-3-(2H)furanone in 2 ml of DME was added 1 ml lithium hydroxide solution. After 4 hours at room temperature, the reaction was concentrated to dryness and azeotroped with toluene (3×) to remove excess water. The residue was dissolved in 5 ml of DMF and 414 mg (6.10 mmol) of imidazole and 465 mg (3.05 mmol) of t-butyldimethylsilyl chloride was added. After two days at room temperature, 1 ml of methanol was added to the reaction and after 1 hour the solution was evaporated to dryness. The residue was partitioned between saturated NH₄Cl solution (aq) and washed with ethyl acetate which was dried, filtered and concentrated to give an oil which was a mixture of product and the furanone starting material. This material was carried on crude into the next reaction.

### Step 6: Preparation of N-(2(R)-hydroxy-1(S)-indanyl)-2(R)-phenylmethyl-4(S)-(t-butyldimethyl-silyloxy-5-(2-(3(S)-N′-(t-butylcarboxamido)-(4aS,8aS)-decahydroisoquinoline)yl)-pentaneamide

The crude product of step 5, above, was dissolved in 3 ml of DMF along with 47 mg (0.246 mmol) of EDC, 33 mg (0.246 mmol) of HOBT and 37 mg of 2(R)-hydroxy-1(S)-aminoindane. The pH of the solution was adjusted to 8.5-9.0 with triethylamine and after 18 hours it was worked up by concentrating to dryness, dissolving the residue in 10% aq. citric acid solution and washing the aqueous layer with ethyl acetate. The organic layer was dried, filtered and concentrated and the resultant oil was chromatographed (SiO₂, 30% EtOAc/Hexane) to yield the title compound.

### Step 7: Preparation of N-(2(R)-hydroxy-1(S)-indanyl)-2(R)-phenylmethyl-4(S)-hydroxy-5-(2-(3(S)-N′-(t-butylcarboxamido)-(4aS,8aS)-decahydroisoquinoline)yl)-pentaneamide

The product from step 6, above, was dissolved in 1 ml of THF and 1 ml of a 1M solution of tetrabutylammonium fluoride in THF was added. After 18 hr at room temperature the reaction was diluted with 20 ml of saturated NaHcO₃ solution (aq) and the product was extracted into ethyl acetate which was dried, filtered and concentrated to give a foam. The resultant material was chromatographed on a prep plate (0.5 mm, 5% MeOH/CHCl₃) and the title product isolated in the usual manner as a solid with mp 105°-107° C.

### Example 5

### Preparation of N-(2(R)-hydroxy-1(S)-indanyl)-2(R)-phenylmethyl-4(S)-amino-5-(2-(3(S)-N′-(t-butylcarboxamido)-(4aS,8aS)-decahydroisoquinoline)yl)-pentaneamide

### Step 1: Preparation of 5(S)-((t-butyl-dimethyl-silyloxy)methyl)-3 (R)-phenylmethyl-N-BOC-2-pyrrolidinone

A solution of 5(S)-((t-butyl-dimethyl-silyloxy)methyl)-N-BOC-2-pyrrolidinone (400 mg, 1.26 mmol) in 2 ml of THF was added to a precooled (−78° C.) 1M solution of lithium hexamethyldisilazide (1.3 ml) in 5 ml of THF. After 45 min, 0.15 ml of benzyl bromide (1.3 mmol) was added and the stirring was continued. After 5 h the reaction was worked up by pouring into a separatory funnel containing 30 ml of an aqueous 10% citric solution. The aqueous layer was extracted (2×30 ml EtOAc) which was backwashed with brine (50 ml) dried, filtered and concentrated to an oil. The residue was chromatographed (SiO₂, 20% EtOAc/Hexane) to afford the product as an oil.

### Step 2: Preparation of 5(S)-hydroxymethyl-3(R)-phenylmethyl-2-pyrrolidinone

To 130 mg (0.34 mmol) of 5(S)-((t-butyl-dimethylsilyloxy)methyl)-3(R)-phenylmethyl-N-BOC-2-pyrrolidinone in 5 ml of acetonitrile was added 0.1 ml of a solution of 48% HF in water. After 3 hr at room temperature the reaction was concentrated to dryness and diluted with 30 ml of an aqueous 10% NaHCO₃solution. This was extracted with EtOAc (2×30 ml), dried filtered and concentrated to afford the crude product.

### Step 3: Preparation of 5(S)-(methanesulfonyloxy)-methyl-3(R),phenylmethyl-2-pyrrolidinone

To a solution of the crude product from Step 2, in 5 ml of methylene chloride cooled to 0° C. was added triethylamine (42 mg, 0.41 mmol) and methanesulfonyl chloride (47 mg, 0.41 mmol). The reaction was slowly allowed to warm to room temperature and was stirred for 18 hr after which time it was diluted with ml of methylene chloride, washed with 30 ml of 10% citric acid solution, dried filtered and concentrated to afford the product as an oil.

### Step 4: Preparation of 5(S)-(2-(3(S)-N-(t-butylcarboxamido)-(4aS,8aS)-(decahydroisoquinoline)yl)- methyl)-3(R)-phenylmethyl-2-pyrrolidinone

To a solution of 380 mg (1.34 mmol) of 5(S)-(methanesulfonyloxy)methyl-3(R)-phenylmethyl-2pyrrolidinone in 20 ml of isopropanol was added 350 mg of potassium carbonate and 360 mg of N-t-butyl-(4aS,8aS)-(decahydroisoquinoline)-3(S)-carboxamide and the reaction was heated to 85° C. After 18 hr the cooled reaction was filtered through celite, evaporated to dryness and the residue was dissolved in water which was extracted with EtOAc (2×50 ml). The organics were dried, filtered and concentrated, and the residue was chromatographed (SiO₂, 50/50 EtOAc/Hexane) to afford the product as an oil.

### Step 5: Preparation of 5(S)-(2-(3(S)-N′-(t-butylcarboxamido)-(4aS,8aS)-(decahydroisoquinoline)yl)-methyl)-3(R)-phenylmethyl-N-BOC-2-pyrrolidinone

To a solution of the product from step 4, above, (260 mg, 0.611 mmol) in 10 ml of methylene chloride was added dimethylaminopyridine (74 mg, 0.6 mmol) and 133 mg (0.61 mmol) of BOC-anhydride. After hr at room temperature the reaction was worked up by diluting with 30 ml of methylene chloride and the organics washed with 30 ml of 10% citric acid solution, brine (30 ml) dried, filtered and concentrated to afford an oil. Chromatography (SiO₂, 40% EtOAc/Hexane) gave the title compound.

### Step 6: Preparation of 5-(2-(3(S)-N′-(t-butylcarboxamido)-(4aS,8aS)-decahydroisoquinoline)yl)-4(S)-[(1′,1′)-(dimethylethorycarbonyl)-amino]-2(R)-phenylmethyl-pentanoic acid

To a solution of the product of step 5, above, (260 mg, 0.495 mmol) dissolved in 3 ml of dimethoxyethane was added 1.5 ml of a 1M solution of aqueous lithium hydroxide (1.5 mmol). The reaction was worked up after 2 hr by concentrating to dryness, dissolving the residue in saturated aqueous ammonium chloride solution and the aqueous phase was washed with ethyl acetate (2×50 ml) which was dried, filtered and concentrated to afford the crude acid.

### Step 7: Preparation of N-(2(R)-hydroxy-1(S)-indanyl)-2(R)-phenylmethyl-4(S)-[(1′,1′)-(dimethylethoxycarbonyl)amino]-5-(2-(3(S)-N′-(t-butylcarboxamido)-(4aS,8aS)-decahydroisoquinoline)yl)-pentaneamide

To a solution of the product of step 6, above, (260 mg, 0.49 mmol) in methylene chloride was added EDC (94 mg, 0.49 mmol), HOBT(66 mg, 0.49 mmol), 2(R)-hydroxy-1(S)-aminoindane (73 mg, 0.49 mmol) and the pH of the reaction was adjusted to 8.5-9.0 using triethylamine. After 5 hr at room temperature the reaction was worked up by diluting with 50 ml of methylene chloride and washing the organics with saturated aqueous ammonium chloride solution. The organic phase was dried, filtered and concentrated and the residue was chromatographed to afford the title compound as a foam.

### Step 8: Preparation of N-(2(R)-hydroxy-1(S)-indanyl)-2(R)-phenylmethyl-4(S)-hydroxy-5-(2-(3(S)-N′-(t-butylcarboxamido)-(4aS,8aS)-decahydroisoquinoline)yl)-pentaneamide

To a solution of the product of step 7, above, (180 mg, 0.28 mmol) in 5 ml of methylene chloride cooled to 0° C. was added 1 ml of trifluoroacetic acid. After 4 hr the reaction was worked up by concentrating to dryness and the residue was dissolved in 50 ml of methylene chloride and washed with 10% aqueous NaHcO₃ solution. The organic layer was dried, filtered and concentrated to give the product as a solid which was chromatographed (SiO₂, 7% MeOH/CH₂Cl₂) to afford the title compound, mp=92°-95° C.

### Example 6

### Preparation of N-(2(R)-hydroxy-1(S)-indanyl)-2(R)-phenylmethyl-4(S)-hydroxy-5-(1-(4-carbobenzyloxy-2-(S)-N′-(t-butylcarboxamido)-piperazinyl))-pentaneamide

Employing substantially the same procedure used in Example 1, but substituting N-t-butyl-4-CBZ-piperazine-2(S)-carboxamide for N-t-butyl-4(S)-phenoxy-L-prolineamide used in step 9 therein, the title compound was obtained.

### Example 7

### Preparation of N″-(N-(2-pyridyl)-valyl)-2(R)-phenylmethyl-4(S)-hydroxy-5-(2-(3(S)-(N′-t-butylcarboxamido)-(4aS,8aS)-decahydroisoquinoline)yl)pentaneamide

Employing substantially the same procedure used in Example 4, but substituting N-2-pyridylvaline for the 2(R)-hydroxy-1(S)aminoindane used in step 6 therein, the title compound was obtained.

### Example 8

### Preparation of N-(2(R)-hydroxy-1(S)-indanyl)-2(R)-phenylmethyl-4(S)-hydroxy-5-(2(S)-(N,-t-butyl-3-phenyl-propionamide)amino)-pentaneamide

Employing substantially the same procedure used in Example 1, but substituting N-t-butyl-phenylalanine amide for the N′-t-butyl-4(S)-phenoxy-L-prolineamide used in step 9 therein, the title compound is obtained.

### Example 9

### Preparation of N-(4(S)-3,4-dihydro-1H-2,2-dioxobenzothiopyranyl)-2(R)-phenylmethyl-4(S)-hydroxy-5-(2-(3(S)-N′-(t-butylcarboxamido)-(4aS,8aS)-decahydroisoquinoline)yl)-pentaneamide

### Step 1: Preparation of N-(4(S)-3,4-dihydro-1H-benzothiopyranyl)-2(R)-phenylmethyl-4(S)-hydroxy-5-(Z-(3(S)-t-butylcarboxamido)-(4aS,8aS)-decahydroisoquinoline)yl)-pentaneamide

Employing substantially the same procedure used in Example 4 but substituting 4(S)-amino-3,4-dihydro-1H-benzothiopyran for the 2(R)-hydroxy-1(S)-aminoindane used in step 6 therein, the title compound is obtained.

### Step 2: Preparation of N-(4(S)-3,4-dihydro-1H-2,2-dioxobenzothiopyranyl)-2(R)-phenylmethyl-4(S)-hydroxy-5-(2-(3(S)-t-butylcarboxamido)-(4aS,8aS)-decahydroisoquinoline)yl)-pentaneamide

The compound from step 1 above is dissolved in a 1:1 mixture of methanol and water. To this is added 10 eq. of OXONE and the reaction is stirred at room temperature. When the reaction is complete, it is concentrated to dryness, water is added and extracted with ethyl acetate which is dried, filtered and concentrated to give the title compound.

### Example 10

### Preparation of N-(4(S)-3,4-dihydro-1H-2,2-dioxobenzothiopyranyl)-2(R)-phenylmethyl-4(S)-hydroxy-5-(1-(4-carbobenzyloxy-2(S)-N′-(t-butylcarboxamido)-piperazinyl))-pentaneamide

### Step 1: Preparation of dihydro-5(S)-(1-(4-carbobenzyloxy-2(S)-N′-(t-butylcarboxamido)-piperazinyl)methyl)-3(R)-phenylmethyl-3(2H)-furanone

Employing substantially the same procedure used in Example 4, step 4 but substituting 4-carbobenzyloxy-2(S)-N′-(t-butylcarboxamido)-piperazine for the N′-t-butyl-(4aS,8aS)-(decahydroisoquinoline)-3(S)-carboxamide used therein, the title compound is produced.

### Step 2: Preparation of 2(R)-phenylmethyl-4(S)-(t-butyldimethylsilyloxy)-5-(1-(4-carbobenzyloxy-2(S)-N′-(t-butylcarboxamido)-piperazinyl))-pentanoic acid

Employing substantially the same procedure used in Example 4, step 5 but substituting dihydro-5(S)-(1-(4-carbobenzyloxy-2(S)-N′-(t-butylcarboxamido)-piperazinyl )methyl)-3(R)-phenylmethyl-3(2H)-furanone for the dihydro-5(S)-(2-(3(S)-N′-(t-butylcarboxamido)-(4aS,8aS)-(decahydroisoquinoline)yl)-methyl)-3(R)-phenylmethyl-3(2H) furanone used therein, the title compound is produced.

Step 3: Preparation of N-(4(S)-3,4-dihydro-1H-benzothiopyranyl)-2(R)-phenylmethyl-4(S)-t-butyldimethylsilyloxy)-5-(1-(4-carbobenzyloxy-2(S)-N′-(t-butylcarboxamido)-piperazinyl))-pentaneamide

The crude 2(R)-phenylmethyl-4(S)-(t-butyldimethylsilyloxy)-5-(1-(4-carbobenzyloxy-2(S)-N′-(t-butylcarboxamido)-piperazinyl))-pentanoic acid is dissolved in 3 ml of DMF along with 1 eq of EDC, 1 eq of HOBT and 1 eq of 4(S)-amino-3,4-dihydro-1H-benzothiopyran. The pH of the solution is adjusted to 8.5-9.0 with triethylamine and after 18 hours it is worked up by concentrating to dryness, dissolving the residue in 10% aq citric acid solution and washing the aqueous layer with ethyl acetate. The organic layer is dried, filtered and concentrated and the resultant residue is chromatographed to yield the title product.

### Step 4: Preparation of N-(4(S)-3,4-dihydro-1benzothiopyranyl)-2(R)-phenylmethyl-4(S)-hydroxy)-5-(1-(4-carbobenzyloxy-2(S)-(t-butylcarboxamide)-piperazinyl))-pentaneamide

The product from step 3 above is dissolved in 1 ml of THF and 1 ml of a 1M solution of tetrabutylammonium fluoride in THF is added. After 18 hr at room temperature the reaction is diluted with 20 ml of saturated NaHcO₃ solution (aq) and the product is extracted into ethyl acetate which is dried, filtered and concentrated to give a residue. The residue is chromatographed to afford the product.

### Step 5: Preparation of N-(4(S)-3,4-dihydro-1H-2,2-dioxobenzothiopyranyl)-2(R)-phenylmethyl-4(S)-hydroxy-5-(1-(4-carbobenzyloxy-2(S)-N′-(t-butylcarboxamido)-piperazinyl))-pentaneamide

The compound from step 4 above is dissolved in a 1:1 mixture of methanol and water. To this is added 10 eq of OXONE and the reaction is stirred at room temperature. When the reaction is complete, it is concentrated to dryness, water is added and extracted with ethyl acetate which is dried, filtered and concentrated to give the title compound.

### Example 11

### Preparation of N-(2(R)-hydroxy-1(S)-indanyl)-2(R)-((4-((2-hydroxy)ethoxy)phenyl)methyl)-4(S)-hydroxy-5-(2-(3-(S)-N′-(t-butylcarboxamido)-(4aS,8aS)-decahydroisoquinoline)yl)-pentaneamide

### Step 1: Preparation of N-(2(R)-hydroxy-1(S)-indanyl)-2(R)-((4-(2-allyloxy)phenyl)methyl)-4(S)-hydroxy-5-(2-(3(S)-t-butylcarboxamido)-(4aS,8aS)-decahydroisoquinoline)yl)-pentaneamide

To a solution of N-(2(R)-hydroxy-1(S)-indanyl)-2(R)-((4-hydroxyphenyl)methyl)-4(S)-hydroxy-5-(2-(3(S)-t-butylcarboxamido)-(4aS,8aS)-decahydroisoquinoline)yl)-pentaneamide in dioxane is added 6 eq of allyl bromide and 6 eq of cesium carbonate. The reaction is heated to 90° C. When the reaction is complete, the precipitate is filtered off, the dioxane is concentrated to dryness and the residue is diluted with water which is washed with ethyl acetate. The organic phase is dried, filtered and concentrated to afford the product.

### Step 2: Preparation of N-(2(R)-hydroxy-1(S)-indanyl)-2(R)-((4-((2-hydroxy)ethoxy)phenyl)methyl)-4(S)-hydroxy-5-(2-(3(S)-N′-(t-butylcarboxamido)-(4aS,8aS)-decahydroisoquinoline)yl)-pentaneamide

The product from step 1 above is dissolved in methanol, 1 eq of p-toluenesulfonic acid is added and the reaction is cooled to −78° C. Excess ozone is bubbled through the reaction until a blue color persists. The flask is purged with nitrogen to remove any ozone and excess sodium borohydride solution is added. The reaction is warmed to room temperature and then saturated NaHCO₃solution is added. The methanol is concentrated off on the rotoevaporater and the aqueous residue is washed with ethyl acetate which is dried, filtered and concentrated to afford the title compound.

### Example 12

### Preparation of N-(2(R)-hydroxy-1(S)-indanyl)-2(R)-((4-((2-hydroxy)ethoxy)-phenyl)methyl)-4(S)-hydroxy-5-(1-(4-carbobenzyloxy-2(S)-N′-(t-butylcarboxamido)-piperazinyl))-pentaneamide

Employing substantially the same procedure used in Example 11 but substituting N-(2(R)-hydroxy-1(S)-indanyl)-2(R)-(-(4-hydroxyphenyl)methyl)-4(S)-hydroxy -5-(1-(4-carbobenzyloxy-2(S)-(t-butylcarboxamido)-piperazinyl)-pentaneamide for the N-(2(R)-hydroxy-1(S)-indanyl)-2(R)-((4-hydroxyphenyl)methyl)-4(S)-hydroxy-5-(2-(3(S)-t-butylcarboxamido)-(4aS,8aS)-decahydroisoquinoline)yl)-pentaneamide used therein, the title compound is obtained.

### Example 13

### Preparation of N-(2(R)-hydroxy-1(S)-indanyl)-2(R)-((4-(2-(4-morpholinyl)ethoxy)phenyl)methyl)-4(S)-hydroxy-5-(2-(3(S)-N′-(t-butylcarboxamido)-(4aS,8aS)-decahydroisoquinoline)yl)-pentaneamide

To a solution of N-(2(R)-hydroxy-1(S)-indanyl)-2(R)-((4-hydroxyphenyl)methyl)-4(S)-hydroxy-5-(2-(3(S)-N′-(t-butylcarboxamido)-(4aS,8aS)-decahydroisoquinoline)yl)-pentaneamide in dioxane is added 6 eq of chloroethyl morpholine and 6 eq of cesium carbonate. The reaction is heated to 90° C. When the reaction is complete, the precipitate is filtered off, the dioxane is concentrated to dryness and the residue is diluted with water which is washed with ethyl acetate, The organic phase is dried, filtered and concentrated to afford the title compound.

### Example 14

### Preparation of N-(2(R)-hydroxy-1(S)-indanyl)-2(R)-((4-(2-(4-morpholinyl)ethoxy)phenyl)methyl)-4(S)-hydroxy-5-(1-(4-carbobenzyloxy-2(S)-N′-(t-butylcarboxamido)-piperazinyl))-pentaneamide

To a solution of N-(2(R)-hydroxy-1(S)-indanyl)-2(R)-((4-hydroxyphenyl)methyl)-4(S)-hydroxy-5-(1-(4-carbobenzyloxy-2(S)-(t-butylcarboxamido)-piperazinyl)-pentaneamide in dioxane is added 6 eq of chloroethyl morpholine and 6 eq of cesium carbonate. The reaction is heated to 90° C. When the reaction is complete, the precipitate is filtered off, the dioxane is concentrated to dryness and the residue is diluted with water which is washed with ethyl acetate. The organic phase is dried, filtered and concentrated to afford the title compound.

### Example 15

### Preparation of N-(2(R)-hydroxy-1(S)-indanyl)-2(R)-phenylmethyl-4(S)-hydroxy-5-(1-(4-(3-pyridylmethyl)-2(S)-N′-(t-butylcarboxamido)-piperazinyl))-pentaneamide

### Step 1: Preparation of dihydro-5(S)-((trifluoromethanesulfonyl)oxymethyl)-3(R)-phenylmethyl-3(2H)-furanone

To a solution of 18.4 g (89.2 mmol) of dihydro-5(S)-(hydroxymethyl)-3(R)-phenylmethyl-3(2H)-furanone in 350 mL of methylene chloride cooled to 0° C. was added 13.51 mL 2,6-lutidine (115.98 mmol) followed by a dropwise addition of 16.51 mL of trifluoromethanesulfonic anhydride (98.1 mmol). After 1.5 hours at 0° C., the reaction was poured into a mixture of 300 mL ice/brine and stirred for 0.5 hours. The aqueous layer was then extracted with methylene chloride (3×150 mL), the organic layers were washed with 10% HCl (2×75 mL), saturated NaHCO₃(100 mL), water (100 mL), dried over MgSO₄, filtered and concentrated to give a solid residue. Purification via flash column chromatography (120×150 mm column, gradient elution of hexanes:EtOAc, 4:1 to 3:1) afforded the title product; mp 53°-54° C.

### Step 2: Preparation of 4-(1,1-dimethylethyl)-1-(phenylmethyl)-1,2(S),4-piperazinetricarboxylate

The title compound was prepared following the procedure of Bigge, C. F.; Hays, S. J.; Novak, P. M.; Drummond, J. T.; Johnson, G.; Bobovski, T. P. Tetrahedron Lett. 1989, 30, 5193; starting with 2(S)-piperazinecarboxylic acid. (see Felder, E.; Maffei, S.; Pietra, S.; Pitre, D.; Helv. Chim. Acta 1960, 117, 888.

### Step 3: Preparation of N-t-butyl-4-(1,1-dimethylethoxycarbonylamino)-1-(phenylmethylcarbonylamino)-piperazine-2(S)-carboxamide

To 9.90 g (27.16 mmol) of 4-(1,1-dimethylethyl)-1-(phenylmethyl)-1,2(S),4-piperazinetricarboxylate dissolved in 75 mL of DMF and cooled to 0° C. was added 5.73 g (29.88 mmol) of EDC, 4.03 g (29.88 mmol) of HOBt, 3.14 mL (29.88 mmol) of t-butylamine, and finally 4.16 mL (29.88 mmol) of triethylamine. The reaction mixture was stirred for 18 hours and the reaction volume was concentrated by half. The mixture was then diluted with 600 mL of EtOAc and washed with 10% HCl (2×75 mL), saturated NaHCO₃(1×75 mL), water (3×75 mL) and brine (1×50 mL), dried over MgSO₄ and concentrated to a solid. This solid was triturated with EtOAc: hexane (1:2) and filtered to provide the title product as a white solid; mp 134°-135° C.

### Step 4: Preparation of N-t-butyl-4-(1,1-dimethylethoxycarbonylamino)piperazine-2(S)-carboxamide

To 1.20 g (2.86 mmol) of N-t-butyl-4-(1,1-dimethylethoxycarbonylamino)-1-(phenylmethylcarbonylamino)piperazine-2(S)-carboxamide and 1.1 g (0.086 mmol) of 10% Pd/C was added 15 mL of methanol. The vessel was charged with hydrogen and the reaction stirred for hours, filtered through celite and washed with ethanol. The solvents were removed in vacuo to provide the title product as a foam.

¹H NMR (300 MHz, CDCl₃) δ 6.65 (br, 1H), 4.10 (m, 1H), 3.81 (br, 1H), 3.21 (dd, J=18 and 7 Hz, 1H), 3.02-2.70 (m, 4H), 2.10-2.0 (br, 1H), 1.50 (s, 9H), 1.41(s, 9H).

### Step 5: Preparation of dihydro-5(S)-(4-(1,1-dimethylethoxycarbonylamino))-2(S)-N-(t-butylcarboxamido)-piperazinyl)methyl)-3(R)-phenylmethyl-3(2H)-furanone

To a solution of 22.40 g (0.0662 mol) dihydro-5(S)-((trifluoromethanesulfonyl)oxymethyl)-3(R)-phenylmethyl-3(2H)-furanone (prep in step 1) and 18.0 g (0.063 mol) of n-t-butyl-4-(1,1-dimethylethoxycarbonylamino)piperazine-2(S)-carboxamide dissolved in 180 mL of isopropanol was added 11.53 mL (0.0662 mol) of N,N-diisopropylethylamine. After 2.5 hours another 1.2 g of dihydro-5(S)-((trifluoromethanesulfonyl)oxymethyl)-3(R)-phenylmethyl-3(2H)-furanone was added. The reaction was complete by thin layer chromatography (tlc) after 3.5 hours and was concentrated to a thick oil. Trituration with EtOAc:hexanes (1:2, 200 mL) provided a white solid which was filtered and discarded. The oil was purified by flash column chromatography (120×150 mm column, EtOAc:hexanes gradient elution 1:1, 2:1, 3:1 to all EtOAc) to afford the title compound.

¹H NMR (400 MHz, CDCl₃) δ 7.34-7.17(m, 5H), 6.31 (br s, 1H), 4.38 (br m, 1H), 3.96-3.92 (m, 1H), 3.79 (br m, 1H), 3.16 (dd, J=13.6 and 4.4 Hz, 1H), 3.08-2.99 (m, 3H), 2.90-2.82 (m, 1H), 2.80 (dd, J=13.5 and 8.9 Hz, 1H), 2.78 (m, 1H), 2.67-2.61 (m,1H), 2.58-2.49 (m, 1H), 2.38-2.32 (m,1H), 2.32-2.04 (m, 1H), 1.99-1.92 (m, 1H,) 1.45 (s, 9H), 1.29 (s, 9H).

### Step 6: Preparation of 2(R)-phenylmethyl-4(S)-(t-butyldimethylsilyloxy)-5-(1-(4-(1,1-dimethylethoxycarbonyl-amino)))-2(S)-N-(t-butylcarboxamido)-piperazinyl))-pentaneamide

To 25.50 g (52.50 mmol) of dihydro-5(S)-(4-(1,1-dimethylethoxycarbonylamino))-2(S)-N-(t-butylcarboxamido)-piperazinyl)methyl)-3(R)-phenylmethyl-3(2H)-furanone dissolved in 120 mL DME cooled to 0° C. was added a solution of 60 mL of water and 1.512 g (63.01 mmol) of lithium hydroxide. After 0.5 hours the reaction was quenched with the addition of 10% HCl until pH 6 and the solution was concentrated in vacuo. The residue was dissolved in 50 mL water and extracted with EtOAc (4×75 mL) and the organic layers were washed with water (1×20 mL), brine (1×20 mL). The aqueous was back extracted with EtOAc (2×75 mL) and the combined organic layers were dried over MgSO₄ and concentrated to provide a yellow solid. This crude product was dissolved in 100 mL of DMF and 17.87 g (0.262 mol) of imidazole was added, cooled to 0° C. and then 31.50 g (0.21 mol) of t-butyldimethylsilyl chloride was added. This stirred 1 hour at 0° C. and was then warmed to room temperature. After 20 hours the reaction was quenched with 10 mL methanol and concentrated to half the volume. 100 mL of pH 7 buffered water was added and the aqueous was extracted with EtOAc (4×100 mL), the combined organic layers were washed with 10% HCl (2×50 mL), water (3×75 mL), and brine (1×50 mL), dried over MgSO₄ and concentrated to obtain the title compound. This material was used directly in the next step.

### Step 7: Preparation of N-(2(R)-hydroxy-1(S)-indanyl)-2(R)-phenylmethyl-4(S)-(t-butyldimethylsilyloxy)-5-(1-(4-(1,1-dimethylethoxycarbonylamino)))-2(S)-N-(t-butylcarboxamido)-piperazinyl))-pentaneamide

To 27.0 g (0.0446 mol) of the crude material from step 6 dissolved in 180 mL of DMF and cooled to 0° C. was added 8.98 g (0.0468 mol) of EDC, 6.32 g (0.0468 mol) of HOBt, and 7.31 g (0.049 mol) aminohydroxy indane. Triethylamine (6.52 mL, 0.0468 mol) was added and the reaction stirred at 0° C. for 2 hours, room temperature for 16 hours and was quenched by diluting with 500 mL of EtOAc. The organic layer was washed with 10% HCl (2×100 mL), saturated NaHCO₃(1×100 mL), water (3×150 mL), brine (1×75 mL), dried over MgSO₄ and concentrated to yield the title compound as a white foam.

¹H NMR (400 MHz, CDCl₃) δ 7.4-7.17 (m, 9H), 6.51 (br s, 1H), 5.79 (br s, 1H), 5.23 (m, 1H), 4.23 (br s, 1H), 4.06 (m, 1H), 3.96-3.84 (m, 2H), 3.07-2.78 (m, 8H), 3.65 (dd, J=9.6 and 4.1 Hz, 1H), 2.56-2.44 (m, 2H), 2.29 (dd, J=12.0 and 4.5 Hz, 1H), 2.17-2.09 (m, 1H), 1.79 (br s, 1H), 1.44 (s, 9H), 1.35 (s, 9H), 1.10 (s, 1H), 0.84 (s, 9H), 0.12 (s, 3H), 0.08 (s, 3H).

### Step 8: Preparation of N-(2(R)-hydroxy-1(S)-indanyl)-2(R)-phenylmethyl-4(S)-(hydroxy)-5-(1-(4-(1,1-dimethylethoxycarbonylamino)))-2(S)-N-(t-butylcarboxamido)-piperazinyl))- pentaneamide

To 32.20 g (0.0437 mol) of N-(2(R)-hydroxy-1-(S)-indanyl)-2(R)-phenylmethyl-4(S)-(t-butyldimethylsilyloxy)-5-(1-(4-(1,1-dimethylethoxycarbonylamino)))-2(S)-N-(t-butylcarboxamido)-piperazinyl))-pentaneamide was added 437 mL (0.437 mol) of tetrabutylammonium fluoride (1.0M solution in THF, Aldrich). The reaction stirred for 18 hours and was then concentrated to 200 mL and diluted with 700 mL of EtOAc. This was washed with water (2×100 mL), brine (1×50 mL) and the aqueous layers were back extracted with EtOAc (2×200 mL). The combined organic layers were dried over MgSO₄ and concentrated to an oil. Purification via flash column chromatography (120×150 mm column, gradient elution CH₂Cl₂: CHCl₃/saturated with NH_{3:} methanol, increasing methanol from 1%, 1.5%, 2%) afforded the title compound as a white foam.

¹H NMR (400 MHz, CDCl₃) δ7.31-7.11 (m, 9H), 6.41 (br s, 1H), 6.23 (d, J=8.6 Hz, 1H), 5.25 (dd, J=8.6 and 4.7 Hz, 1H), 4.21 (m, 1H), 3.83-3.82 (m, 2H), 3.78-3.61 (m, 2H), 3.22-3.19 (m, 2H), 3.03-2.78 (m, 8H), 2.62-2.58 (m, 1H), 2.41-2.35 (m, 2H), 2.04-2.02 (m, 1H), 1.57-1.50 (m, 1H), 1.45 (s, 9H), 1.32 (s, 9H).

### Step 9: Preparation of N-(2(R)-hydroxy-1(S)-indanyl)-2(R)-phenylmethyl-4(S)-(hydroxy)-5-(1-(2(S)- N-(t-butylcarboxamido)-piperazinyl)-pentaneamide

To 21.15 g (0.034 mol) of N-(2(R)-hydroxy-1(S)-indanyl)-2(R)-phenylmethyl-4(S)-(hydroxy)-5-(1-(4-(1,1-dimethylethoxycarbonylamino)))-2(S)-N-(t-butylcarboxamido)-piperazinyl))-pentaneamide dissolved in 350 mL of methylene chloride and cooled to 0° C. was added 22.43 mL (0.204 mol) 2,6-lutidine and then 32.85 mL (0.170 mol) of trimethylsilyltriflate over 5 minutes. After 0.5 hours the reaction was quenched with 10% HCl (80 mL) and this stirred 0.5 hours. To this was added 100 mL of saturated NaHCO₃ and then solid NaHCO₃ until pH 8. The aqueous layer was then extracted with EtOAc (4×100 mL).and the combined organic layers were washed with water (1×50 mL), brine (1×75 mL), dried over MgSO₄ and concentrated. The residue was purified via column chromatography (120×150 mm column, gradient elution CH₂Cl₂:CHCl₃ saturated with NH₃:MeOH, slowly increasing methanol 2%, 3%, 4%, 5%, 6%, to 10%). This provided the title product as a white foam.

¹H NMR (400 MHz, CDCl₃) δ 7.53 (s, 1H), 7.29-7.09 (m, 9H), 6.52 (d=8.3 Hz, 1H), 5.24 (dd, J=8.2 and 4.9 Hz, 1H), 4.23 (dd, 3=4.7 and 4.03 Hz, 1H), 4.25-4.00 (br s, 1H), 3.83-3.81 (m, 1H), 3.03-2.88 (m, 4H), 2.82-2.73 (m, 7H), 2.50-1.60 (br s, 2H), 2.45 (d, J=6.2 Hz, 2H), 2.32-2.29 (m, 1H), 1.98 (m, 1H), 1.51 (m, 1H) , 1.33 (s, 9H).

### Step 10: Preparation of N-(2(R)-hydroxy-1(S)-indanyl)-2(R)-phenylmethyl-4(S)-hydroxy-5-(1-(4-(3-pyridylmethyl)-2(S)-N′-(t-butylcarboxamido)-piperazinyl))-pentaneamide

To 10.0 g (0.019 mol) of N-(2(R)-hydroxy-1(S)-indanyl)-2(R)-phenylmethyl-4(S)-hydroxy)-5-(1-(2(S)-N-(t-butylcarboxamido)-piperazinyl)-pentaneamide and 3.45 g (0.021 mol) of 3-picolyl chloride dissolved in 40 ml of DMF was added 5.85 mL (0.042 mol) of triethylamine. After 3 hours an additional 0.313 g of 3-picolyl chloride was added. After an additional 2 hours the reaction was diluted with 400 mL of EtOAc and washed with water (3×75 mL), brine (1×100 mL), dried over MgSO₄ and concentrated. The residue was triturated with 30 mL of EtOAc and the resulting white precipitate was collected. Further recrystallization from EtOAc provided the title product (mp 167.5°-168° C.)

### Example 16

Employing substantially the same procedure as described in Example 15, but treating the N-(2(R)-hydroxy-1(S)-indanyl)-2(R)-phenylmethyl-4(S)-hydroxy-5-(1-(2(S)-N′-(t-butylcarboxamido)-piperazinyl))-pentaneamide used therein (compound (i) below) with the alkylating agent (ii) indicated below in place of the 3-picolyl chloride used in Step 10 therein, the following products defined by formula (iii) were made:

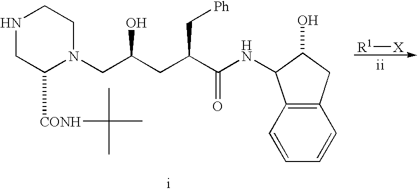

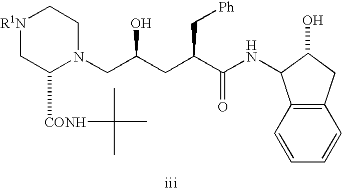

| R¹ | X |
|---|---|
| 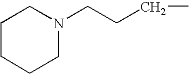 | I |
| 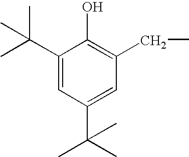 | Cl |
| 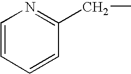 | Cl |
| 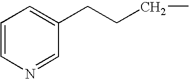 | I |
| CH₃CH₂— | I |
| 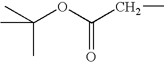 | Br |
| 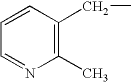 | Cl |
| 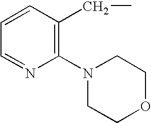 | Cl |
| 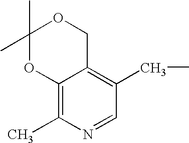 | Cl |
| 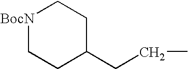 | I |
| 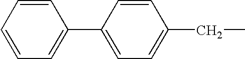 | Cl |
| 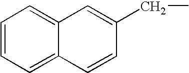 | Cl |
| 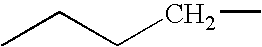 | I |
| 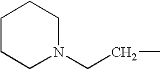 | Cl |
| 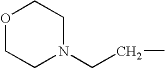 | Cl |
| 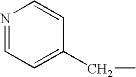 | Cl |
| CH₃O(CH₂CH₂O)₂—CH₂CH₂— | I |
| 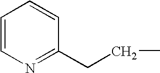 | I |
| 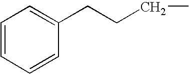 | I |
| 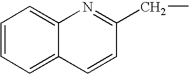 | Cl |
| 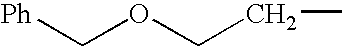 | I |
| 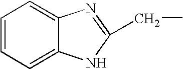 | Cl |
| 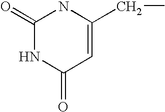 | Cl |
| 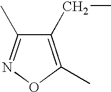 | Cl |
| 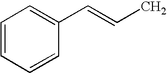 | Cl |
| 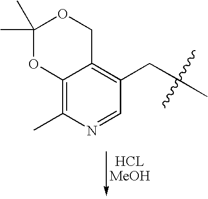 | Cl |
| 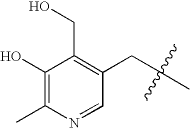 | |
| 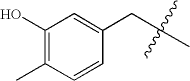 | Br |
| 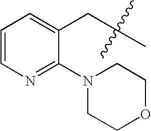 | Cl |
| 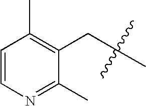 | Cl |
| 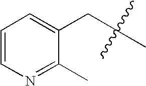 | Cl |
| 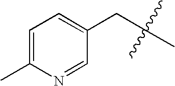 | Cl |
| 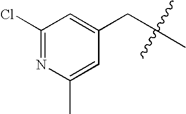 | Cl |
| 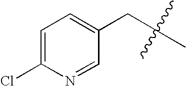 | Cl |
| 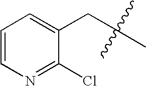 | Cl |
| 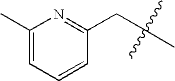 | Cl |
| 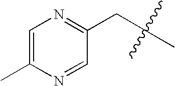 | Cl |
| 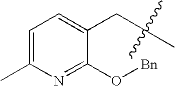 | Cl |
| 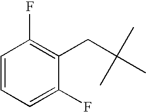 | Br |
| 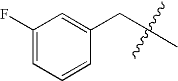 | Br |
| 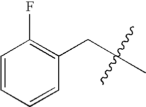 | Br |
| 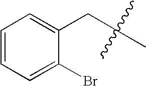 | Br |
| 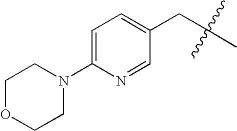 | Cl |
| 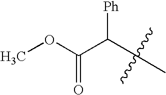 | 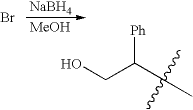 |
| 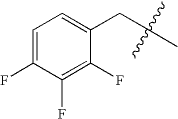 | Br |
| 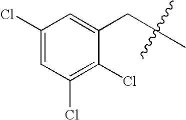 | Br |
| 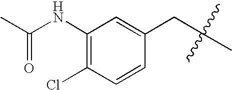 | Br |
| 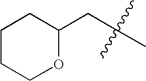 | Br |
| 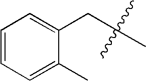 | Br |
| 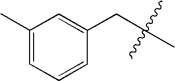 | Br |
| 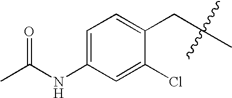 | Br |
| 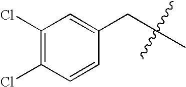 | Cl |
| 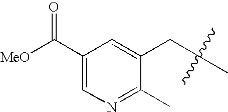 | Cl |
| 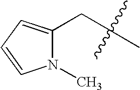 | Cl |
| 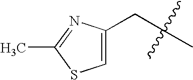 | Cl |
| 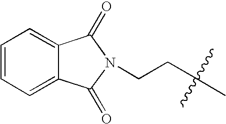 | Br |
| 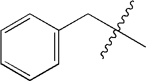 | |
| 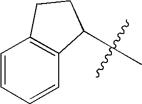 | Br |
| 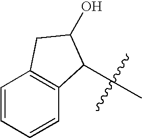 | Br |
| 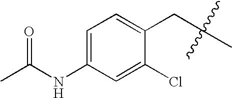 | Cl |
| 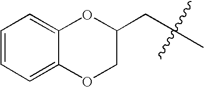 | Br |
| 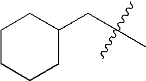 | |

### Example 17

### Preparation of dihydro-5(S)-(tert-butyldimethylsilyl-oxymethyl)-3(2H)-furanone

To a solution of 3.00 g (25.8 mmol) of dihydro-5(S)-(hydroxymethyl)-2(3H)-furanone dissolved in 25 mL of dichloromethane was added 3.51 g (51.6 mmol) of imidazole and then 4.67 g (31.0 mmol) of tert-butyldimethylsilyl chloride. The reaction stirred at room temperature for 8 hours and was quenched with 2 mL of methanol. The mixture was concentrated to an oil and then diluted with 150 mL of ether and washed with 5% HCl(2×10 mL), saturated NaHCO₃(1×10 mL), water (1×10 mL), and brine (1×10 mL), dried over MgSO₄ and concentrated. The residue was purified by flash chromatography (40×150 mm column, gradient elution, hexanes:ethyl/acetate 5:1 to 4:1) to afford the product as a clear oil.

¹H NMR (300 MHz, CDCl₃) δ 4.68-4.60 (m, 1H), 3.89 (dd, J=3.3 and 11.3 Hz, 1H), 3.71 (dd, 3=3.2 and 5411.3 Hz,1H), 2.71-2.45 (m, 2H), 2.35-2.16 (m, 2H), 0.91 (s, 9H), 0.10 (s, 3H), 0.09 (s, 3H).

### Example 18

### Preparation of N-(2(R)-hydroxy-1(S)-indanyl)-2(R)-phenylmethyl-4(S)-hydroxy-5-(1-(4-(4-bromo-2-thiophenemethyl)-2(S)-N′-(t-butylcarboxamido)-piperazinyl))pentaneamide

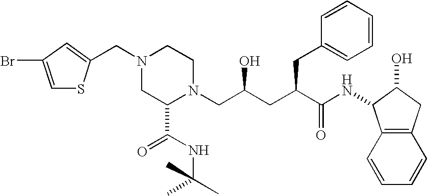

To a solution of 50 mg (0.096 mmol) of N-(2(R)-hydroxy-1(S)-indanyl-2(R)-phenylmethyl-4(S)-hydroxy-5-(1-(2-(S)-N′-(t-butylcarboxamide)-piperazinyl))pentaneamide of Step 9, Example 15, dissolved in 0.4 mL of methanol was added 27.5 mg (0.144 mmol) of 4-bromo-2-thiophene carboxylic aldehyde, 9.0 mg (0.144 mmol) sodium cyanoborohydride and then acetic acid (20 .mu.L) until pH=6. The reaction stirred at room temperature for 8 hour and was quenched with 0.5 mL of 1N HCl. The mixture was concentrated to a white solid and then diluted with 50 mL of ethyl acetate and washed with saturated NaHCO₃(1×5 mL), water (1×5 mL), and brine (1×5 mL), dried over MgSO₄and contrated. The residue was purified by flash column chromatography (15×150 mm column, gradient elution in methylene chloride: chloroform saturated with NH₃: methanol 69:30:1 to 67:30:3 to afford 40.3 mg (60% yield) of the product as a clear oil. An analytical sample was obtained by titration with ethyl acetate and hexanes.

Anal. Calcd for C₃5 H₄5 N₄O₄BrS 0.4 mol H₂O: C, 59.63; H, 6.55; N, 7.95. Found C, 59.66; H, 6.45, N, 7.86.

### Example 19

By substantially the same procedure as described in Example 18, but substituting a different aldehyde (R¹ CHO), the following compounds are prepared.

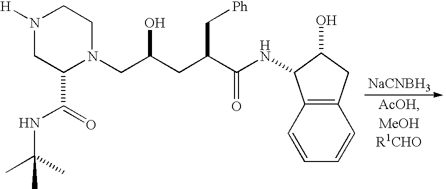

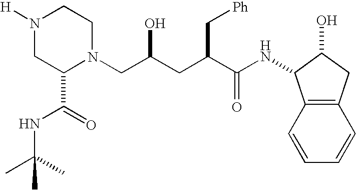

| R¹ | R¹ |
|---|---|
| 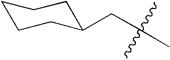 | 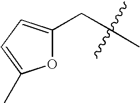 |
| 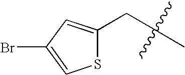 | 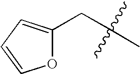 |
| 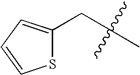 | 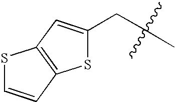 |
| 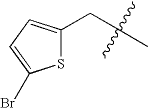 | 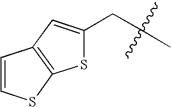 |
| 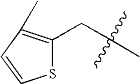 | 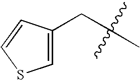 |

The reductive amination reaction of Example 18 is also used to synthesize the following compounds, wherein the 2(R)-phenylmethyl group is modified to a pyridylmethyl group.

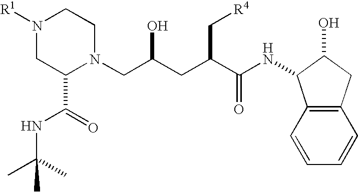

| R¹ | R⁴ |
|---|---|
| 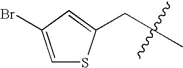 | 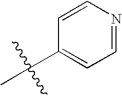 |
| 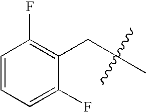 | 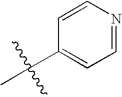 |
| 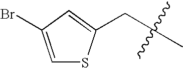 | 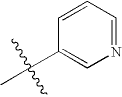 |
| 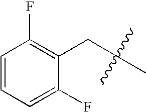 | 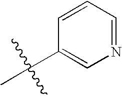 |
| 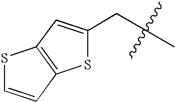 | 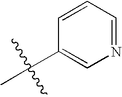 |
| 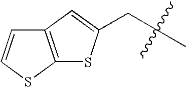 | 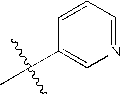 |
| 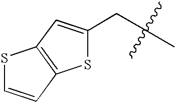 | 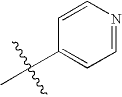 |
| 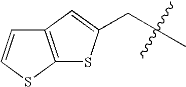 | 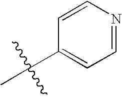 |

Unless defined otherwise, all scientific and technical terms used herein have the same meaning as commonly understood by one of skill in the art to which this invention belongs.

All patents and publications referred to herein are hereby incorporated by reference for all purposes.

The invention has been described with reference to various specific and preferred embodiments and techniques. However, it should be understood that many variations and modifications may be made while remaining within the spirit and scope of the invention.

1. A method according to claim 5, where the disease is

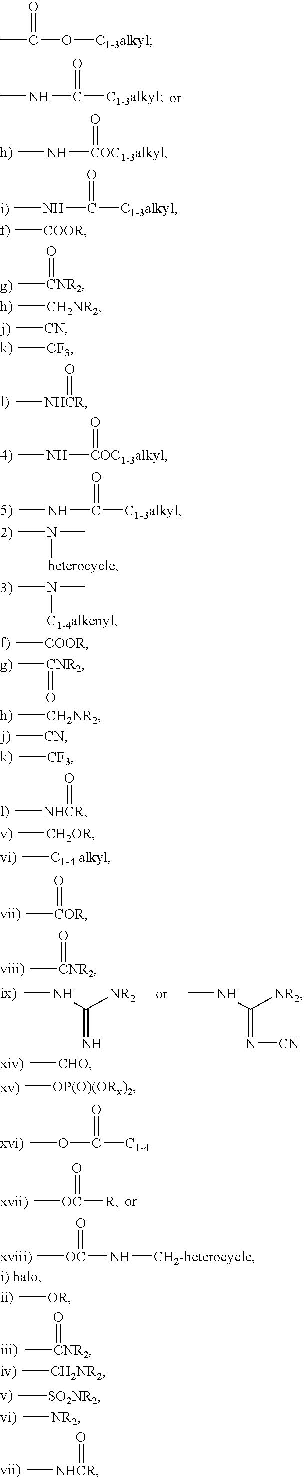

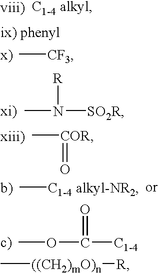

2. A method of treating Alzheimer's disease in a subject in need of such treatment comprising administering to the subject a compound of Formula I, or a pharmaceutically acceptable salt thereof.

3. A method of treating Alzheimer's disease by modulating the activity of beta amyloid converting enzyme, comprising administering to a subject in need of such treatment a compound of Formula I, or a pharmaceutically acceptable salt thereof.

4. The method according to claim 1, further comprising the administration of a P-gp inhibitor, or a pharmaceutically acceptable salt thereof.

5. A method of treating a subject who has, or in preventing a subject from getting, a disease or condition selected from the group consisting of Alzheimer's disease, for helping prevent or delay the onset of Alzheimer's disease, for treating subjects with mild cognitive impairment (MCI) and preventing or delaying the onset of Alzheimer's disease in those who would progress from MCI to AD, for treating Down's syndrome, for treating humans who have Hereditary Cerebral Hemorrhage with Amyloidosis of the Dutch-Type, for treating cerebral amyloid angiopathy and preventing its potential consequences, i.e. single and recurrent lobar hemorrhages, for treating other degenerative dementias, including dementias of mixed vascular and degenerative origin, dementia associated with Parkinson's disease, frontotemporal dementias with parkinsonism (FTDP), dementia associated with progressive supranuclear palsy, dementia associated with cortical basal degeneration, or diffuse Lewy body type of Alzheimer's disease and who is in need of such treatment which includes administration of a therapeutically effective amount of a compound of formula (I), or a pharmaceutically acceptable salt thereof: 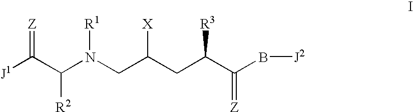
wherein
X is —OH or —NH₂;
Z is —O, —S, or —NH;
R is hydrogen or C₁₋₄ alkyl;
R¹ and R² are independently:
1) hydrogen,
2) —C₁₋₄ alkyl unsubstituted or substituted with one or more of
a) halo,
b) hydroxy,
c) C₁₋₃ alkoxy,
d) aryl unsubstituted or substituted with one or more of C₁₋₄ alkyl, halo, amino, hydroxy or aryl,
e) —W-aryl or —W-benzyl, wherein W is —O—, —S—, or —NH—,
f) a 5-7 membered cycloalkyl group unsubstituted or substituted with one or more of
i) halo,
ii) hydroxy,
iii) C₁₋₃ alkoxy,
iv) aryl,
g) heterocycle unsubstituted or substituted with one or more of hydroxy, oxo, halo, C₁₋₄ alkoxy, C₁₋₄ alkyl optionally substituted with hydroxy; 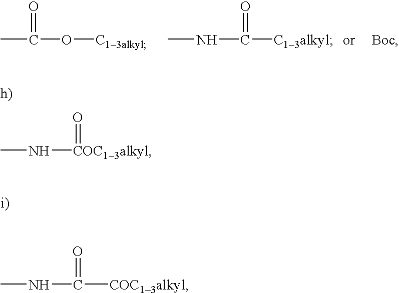
j) —NH—SO₂C₁₋₃alkyl,
k) —NR₂,
l) —COOR, or
m) —((CH₂)ₘO)ₙR wherein m is 2-5 and n is zero, 1, 2 or 3, or
3) aryl, unsubstituted or substituted with one or more of
a) halo,
b) hydroxy,
c) —NO₂ or —NR₂,
d) C₁₋₄alkyl,
e) C₁₋₃alkoxy, unsubstituted or substituted with one or more of —OH or C₁₋₃alkoxy, 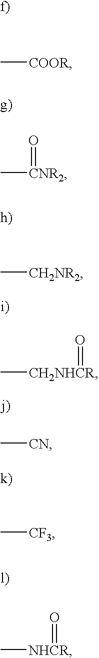
m) aryl C₁₋₃alkoxy,
n) aryl,
o) —NRSO₂R,
p) —OP(O)(ORₓ)₂, or
q) —R⁵, as defined below; or
4) heterocycle unsubstituted or substituted with one or more of hydroxy, oxo, halo, amino, C₁₋₄ alkoxy, C₁₋₄ alkyl optionally substituted with hydroxy; or Boc;
5) carbocyclic unsubstituted or substituted with one or more of halo, amino, hydroxy or C₁₋₄ alkoxy;
R¹ and R² can be joined together to form with the nitrogen to which R¹ is attached a 3 to 10 membered monocyclic or bicyclic saturated ring system which consists of the nitrogen to which R¹ is attached and from 2 to 9 carbon atoms, and is unsubstituted or substituted with
1) hydroxy,
2) C₁₋₄ alkyl unsubstituted or substituted with one or more of
a) halo,
b) hydroxy,
c) C₁₋₃ alkoxy,
d) aryl,
e) a 5-7 membered cycloalkyl group unsubstituted or substituted with one or more of
i) halo,
ii) hydroxy,
iii) C₁₋₃ alkoxy, or
iv) aryl,
f) heterocycle, or
g) —NR₂,
3) C₁₋₃ alkoxy, 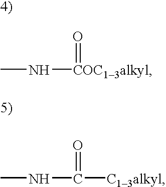
6) —NH—SO₂C₁₋₃ alkyl,
7) heterocycle,
8) —W-aryl, or 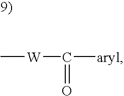
wherein W is defined above; or
R¹ and R² can be joined together to form with the nitrogen to which R¹ is attached a 3 to 10 membered monocyclic or bicyclic saturated ring system which consists of the nitrogen to which R¹ is attached, from 1 to 8 carbon atoms and one or more unsubstituted or substituted heteroatom selected from 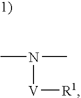
wherein V is absent or 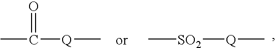
R¹ is defined as above for when R¹ is independent from and not joined to R², and wherein Q is absent or —O—, —NR—, or heterocycle optionally substituted with —C₁₋₄alkyl, 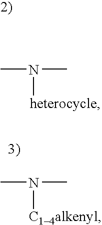
unsubstituted or substituted with aryl, 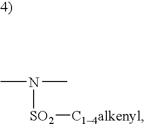
unsubstituted or substituted with aryl, 5) —S(O)p-, wherein p is zero, 1 or 2, or
6) —O—; or
R¹ and R² can be joined together to form with the nitrogen to which R¹ is attached a 3 to 10 membered monocyclic or bicyclic saturated ring system, which consists of the nitrogen to which R¹ is attached and from 2 to 9 carbon atoms, in which the saturated ring system is fused to a phenyl ring and the phenyl ring is unsubstituted or substituted with one or more of
1) halo,
2) C₁₋₃ alkoxy,
3) hydroxy,
4) C₁₋₄ alkyl,
5) —NHR¹, wherein R¹ is defined as above for when R¹ is independent from and not joined to R², or
6) —NH-heterocycle;
R³ is
1) —(CH₂)ᵣ—R⁴, wherein r is zero through 5,
2) C₁₋₄alkenyl-R⁴,
3) C₁₋₄ alkynyl-R⁴;
R⁴ is
1) hydrogen,
2) C₁₋₄ alkyl,
3) C_{5-C}₁₀ cycloalkyl, optionally substituted with hydroxy,
4) C_{6-C}₁₀ aryl, unsubstituted or substituted with one or more of
a) halo,
b) hydroxy,
c) —NO₂ or —NR²,
d) C₁₋₄ alkyl,
e) C₁₋₃ alkoxy, unsubstituted or substituted with one or more of —OH or C₁₋₃ alkoxy, 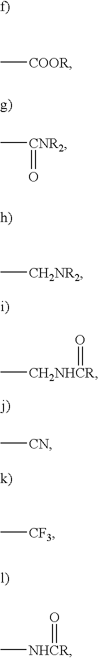
m) aryl C₁₋₃ alkoxy, n) aryl,
o) —NRSO₂R,
p) —OP(O)(ORₓ)₂, or
q) —R⁵, as defined below, or
5) monocyclic or bicyclic heterocyle containing from 1 to 3 heteroatoms chosen from the group consisting of N, O, and S and which is unsubstituted or substituted with R⁵ and optionally with one or more of
a) halo,
b) C₁₋₄ alkyl, or
c) C₁₋₃ alkoxy;
Rₓ is H or aryl;
R⁵ is
1) —W—(CH₂)ₘ—NR⁶R⁷ wherein W is as defined above, m is 2-5, and R⁶ and R⁷ are independently
a) hydrogen,
b) C₁₋₆ alkyl, unsubstituted or substituted with one or more of
i) C₁₋₃ alkoxy,
ii) —OH, or
iii) —NR₂,
c) the same or different and joined together to form a 5-7 member heterocycle, such as morpholino, containing up to two additional heteroatoms selected from 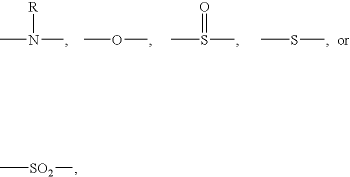
the heterocycle optionally substituted with C₁₋₄ alkyl, or
d) aromatic heterocycle unsubstituted or substituted with one or more of
i) C₁₋₄ alkyl, or
ii) —NR²,
2) —(CH₂)_{q}—NR⁶R⁷ wherein q is 1-5, and R⁶ and R⁷ are defined above, except that R⁶ or R⁷ are not H or unsubstituted C₁₋₆ alkyl, or
3) benzofuryl, indolyl, azacycloalkyl, azabicyclo C₇₋₁₁ cycloalkyl, or benzopiperidinyl, unsubstituted or substituted with C₁₋₄ alkyl;
B is absent, or 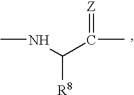
wherein R⁸ is 1) —CH (CH₃)₂,
2) —CH (CH₃)(CH₂CH₃), or
3) -phenyl;
J¹ and J² are independently
1) —YR⁹ wherein Y is —O— or —NH—, and R⁹ is
a) hydrogen,
b) C₁₋₆ alkyl, unsubstituted or substituted with one or more of
i) —NR₂,
ii) —OR,
iii) —NHSO₂C₁₋₄ alkyl,
iv) NHSO₂aryl, or —NHSO₂(dialkylaminoaryl),
v) —CH₂OR,
vi) —C₁₋₄ alkyl, 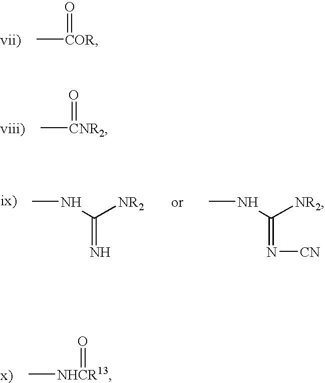
wherein R¹³ is
A) —H
B) —C₁₋₄ alkyl,
C) -aryl,
D) -heterocycle, or
E) —NH—, —O— or —(CH₂)ₙ— wherein n is zero, 1, 2 or 3, substituted with
I) —C₁₋₄ alkyl, unsubstituted or substituted with one or more of aryl or heterocycle, or
II) aryl, unsubstituted or substituted with heterocycle,
xi) —NR₃^{⊕}A^{⊖} wherein A^{⊖} is a counterion,
xii) —NR¹⁰R¹¹ wherein R¹⁰ and R¹¹ are the same or different and are C₁₋₅ alkyl joined together directly to form a 5-7 membered heterocycle containing up to one additional heteroatom selected from —O—, —S—, or —NR—,
xiii) aryl,
xiv) —CHO,
xv) —OP(O)(ORₓ)₂, 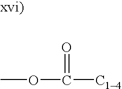
alkyl substituted with one or more of amine or quaternary amine, or —O—((CH₂)ₘO)ₙ—R, or —OP(O)(ORₓ)₂, 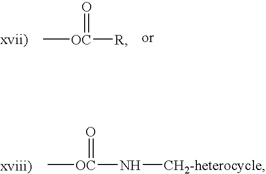
or
c) —((CH₂)ₘO)ₙCH₃ or —((CH₂)ₘO)ₙH, wherein m and n are defined above, or
2) —N(R⁹)ₓ,
3) —NR¹⁰R¹¹ wherein R¹⁰ and R¹¹ are defined above, or 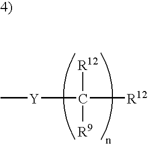
wherein Y, R⁹ and n are defined above; and
R¹² is
1) hydrogen,
2) aryl, unsubstituted or substituted with one or more of
a) R¹⁴, wherein R¹⁴ is 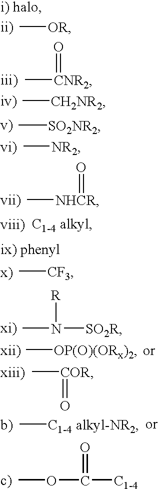
alkyl substituted with one or more of amine or quaternary amine or —OP(O)(ORₓ)₂,
3) heterocycle, such as isochroman, chroman, isothiochroman, thiochroman, benzimidazole, benzothiopyran, oxobenzothiopyran, benzopyran, benzothiopyranylsulfone, benzothiopyranylsulfoxide, the ring or rings being unsubstituted or substituted with one or more of
a) R¹⁴, as defined above,
b) —OC₁₋₄ alkenyl,
c) phenyl-C₁₋₄ alkyl, 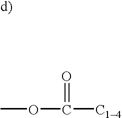
alkyl substituted with one or more of amine or quaternary amine, or —OP(O)(ORₓ)₂, or 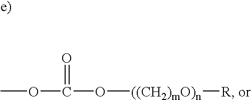
4) A 5 to 7 membered carbocyclic or 7-10 membered bicyclic carbocyclic ring, such as cyclopentane, cyclohexane, indane, norbornane, naphthalene, thiopyran, isothiopyran, or benzopyran, the carbocyclic ring being unsubstituted or substituted with one or more of
a) R¹⁴, as defined above,
b) —CH₂OR,
c) —(CH₂)ₙ—NR₂, C₅₋₁₆alkyl, pyridine, 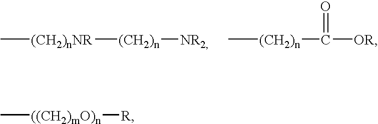
quinuclidiniumyl substituted with R, piperazine-C₁₋₄ alkyl-benzyl substituted one or more with R, or morpholino-C₁₋₄ alkyl-benzyl, 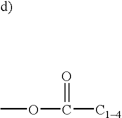
alkyl substituted with one or more of amine or quaternary amine, —OP(ORₓ)₂ or 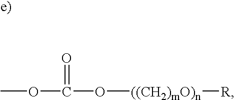
or
f) —C₁₋₄ alkyl-phenyl.

6-9. (Cancelled)

10. A method for inhibiting beta-secretase activity, comprising contacting an effective amount for inhibition of a compound of formula (I): 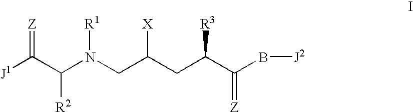
wherein
X is —OH or —NH₂;
Z is —O, —S, or —NH;
R is hydrogen or C₁₋₄ alkyl;
R¹ and R² are independently:
1) hydrogen,
2) —C₁₋₄ alkyl unsubstituted or substituted with one or more of
a) halo,
b) hydroxy,
c) C₁₋₃ alkoxy,
d) aryl unsubstituted or substituted with one or more of C₁₋₄ alkyl, halo, amino, hydroxy or aryl,
e) —W-aryl or —W-benzyl, wherein W is —O—, —S—, or —NH—,
f) a 5-7 membered cycloalkyl group unsubstituted or substituted with one or more of
i) halo,
ii) hydroxy,
iii) C₁₋₃ alkoxy,
iv) aryl,
g) heterocycle unsubstituted or substituted with one or more of hydroxy, oxo, halo, C₁₋₄ alkoxy, C₁₋₄ alkyl optionally substituted with hydroxy; 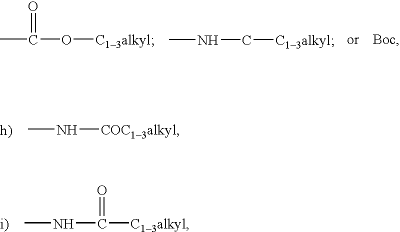
j) —NH—SO₂C₁₋₃alkyl,
k) —NR₂,
l) —COOR, or
m) —((CH₂)ₘO)ₙR wherein m is 2-5 and n is zero, 1, 2 or 3, or
3) aryl, unsubstituted or substituted with one or more of
a) halo,
b) hydroxy,
c) —NO₂ or —NR₂,
d) C₁₋₄alkyl,
e) C₁₋₃alkoxy, unsubstituted or substituted with one or more of —OH or C₁₋₃alkoxy, 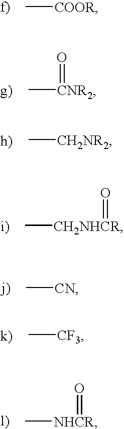
m) aryl C₁₋₃alkoxy,
n) aryl,
o) —NRSO₂R,
p) —OP(O)(ORₓ)₂, or
q) —R⁵, as defined below; or
4) heterocycle unsubstituted or substituted with one or more of hydroxy, oxo, halo, amino, C₁₋₄ alkoxy, C₁₋₄ alkyl optionally substituted with hydroxy; or Boc;
5) carbocyclic unsubstituted or substituted with one or more of halo, amino, hydroxy or C₁₋₄ alkoxy;
R¹ and R² can be joined together to form with the nitrogen to which R¹ is attached a 3 to 10 membered monocyclic or bicyclic saturated ring system which consists of the nitrogen to which R¹ is attached and from 2 to 9 carbon atoms, and is unsubstituted or substituted with
1) hydroxy,
2) C₁₋₄ alkyl unsubstituted or substituted with one or more of
a) halo,
b) hydroxy,
c) C₁₋₃ alkoxy,
d) aryl,
e) a 5-7 membered cycloalkyl group unsubstituted or substituted with one or more of
i) halo,
ii) hydroxy,
iii) C₁₋₃ alkoxy, or
iv) aryl,
f) heterocycle, or
g) —NR₂,
3) C₁₋₃ alkoxy, 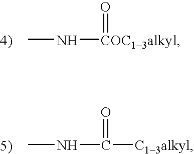
6) —NH—SO₂C₁₋₃ alkyl,
7) heterocycle,
8) —W-aryl, or 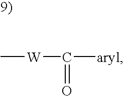
wherein W is defined above; or
R¹ and R² can be joined together to form with the nitrogen to which R¹ is attached a 3 to 10 membered monocyclic or bicyclic saturated ring system which consists of the nitrogen to which R¹ is attached, from 1 to 8 carbon atoms and one or more unsubstituted or substituted heteroatom selected from 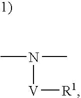
wherein V is absent or 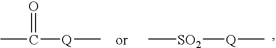
R¹ is defined as above for when R¹ is independent from and not joined to R², and wherein Q is absent or —O—, —NR—, or heterocycle optionally substituted with —C₁₋₄alkyl, 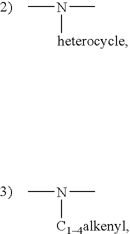
unsubstituted or substituted with aryl, 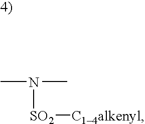
unsubstituted or substituted with aryl, 5) —S(O)p-, wherein p is zero, 1 or 2, or
6) —O—; or
R¹ and R² can be joined together to form with the nitrogen to which R¹ is attached a 3 to 10 membered monocyclic or bicyclic saturated ring system, which consists of the nitrogen to which R¹ is attached and from 2 to 9 carbon atoms, in which the saturated ring system is fused to a phenyl ring and the phenyl ring is unsubstituted or substituted with one or more of
1) halo,
2) C₁₋₃ alkoxy,
3) hydroxy,
4) C₁₋₄ alkyl,
5) —NHR¹, wherein R¹ is defined as above for when R¹ is independent from and not joined to R², or
6) —NH-heterocycle;
R³ is
1) —(CH₂)ᵣ—R⁴, wherein r is zero through 5,
2) C₁₋₄alkenyl-R⁴,
3) C₁₋₄ alkynyl-R⁴;
R⁴ is
1) hydrogen,
2) C₁₋₄ alkyl,
3) C₅-C₁₀ cycloalkyl, optionally substituted with hydroxy,
4) C₆-C₁₀ aryl, unsubstituted or substituted with one or more of
a) halo,
b) hydroxy,
c) —NO₂ or —NR₂,
d) C₁₋₄ alkyl,
e) C₁₋₃ alkoxy, unsubstituted or substituted with one or more of —OH or C₁₋₃ alkoxy, 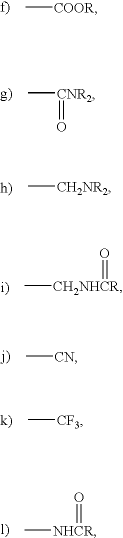
m) aryl C₁₋₃ alkoxy, n) aryl,
o) —NRSO₂R,
P) —OP(O)(ORₓ)₂, or
q) —R⁵, as defined below, or
5) monocyclic or bicyclic heterocyle containing from 1 to 3 heteroatoms chosen from the group consisting of N, O, and S and which is unsubstituted or substituted with R⁵ and optionally with one or more of
a) halo,
b) C₁₋₄ alkyl, or
c) C₁₋₃ alkoxy;
Rₓ is H or aryl;
R⁵ is
1) —W—(CH₂)ₘ—NR⁶R⁷ wherein W is as defined above, m is 2-5, and R⁶ and R⁷ are independently
a) hydrogen,
b) C₁₋₆ alkyl, unsubstituted or substituted with one or more of
i) C₁₋₃ alkoxy,
ii) —OH, or
iii) —NR₂,
c) the same or different and joined together to form a 5-7 member heterocycle, such as morpholino, containing up to two additional heteroatoms selected from 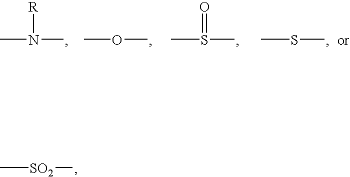
the heterocycle optionally substituted with C₁₋₄ alkyl, or
d) aromatic heterocycle unsubstituted or substituted with one or more of
i) C₁₋₄ alkyl, or
ii) —NR²,
2) —(CH₂)_{q}—NR⁶R⁷ wherein q is 1-5, and R⁶ and R⁷ are defined above, except that R⁶ or R⁷ are not H or unsubstituted C₁₋₆ alkyl, or
3) benzofuryl, indolyl, azacycloalkyl, azabicyclo C₇₋₁₁ cycloalkyl, or benzopiperidinyl, unsubstituted or substituted with C₁₋₄ alkyl;
B is absent, or 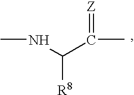
wherein R⁸ is 1) —CH(CH₃)₂,
2) —CH(CH₃)(CH₂CH₃), or
3) -phenyl;
J¹ and J² are independently
1) —YR⁹ wherein Y is —O— or —NH—, and R⁹ is
a) hydrogen,
b) C₁₋₆ alkyl, unsubstituted or substituted with one or more of
i) —NR₂,
ii) —OR,
iii) —NHSO₂C₁₋₄ alkyl,
iv) NHSO₂aryl, or —NHSO₂(dialkylaminoaryl),
v) —CH₂OR,
vi) —C₁₋₄ alkyl, 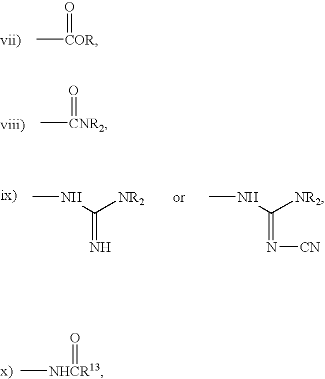
wherein R¹³ is
A) —H
B) —C₁₋₄ alkyl,
C) -aryl,
D) -heterocycle, or
E) —NH—, —O— or —(CH₂)ₙ— wherein n is zero, 1, 2 or 3, substituted with
I) —C₁₋₄ alkyl, unsubstituted or substituted with one or more of aryl or heterocycle, or
II) aryl, unsubstituted or substituted with heterocycle,
xi) —NR₃^{⊕}A^{⊖} wherein A^{⊖} is a counterion,
xii) —NR¹⁰R¹¹ wherein R¹⁰ and R¹¹ are the same or different and are C₁₋₅ alkyl joined together directly to form a 5-7 membered heterocycle containing up to one additional heteroatom selected from —O—, —S—, or —NR—,
xiii) aryl,
xiv) —CHO,
xv) —OP(O)(ORₓ)₂, 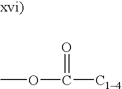
alkyl substituted with one or more of amine or quaternary amine, or —O—((CH₂)ₘO)ₙ—R, or —OP(O)(ORₓ)₂, 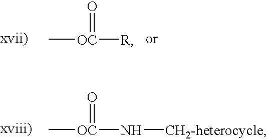
or
c) —((CH₂)ₘO)ₙCH₃ or —((CH₂)ₘO)ₙH, wherein m and n are defined above, or
2) —N(R⁹)ₓ,
3) —NR¹⁰R¹¹ wherein R¹⁰ and R¹¹ are defined above, or 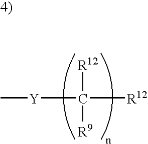
wherein Y, R⁹ and n are defined above; and
R¹² is
1) hydrogen,
2) aryl, unsubstituted or substituted with one or more of
a) R¹⁴, wherein R¹⁴ is 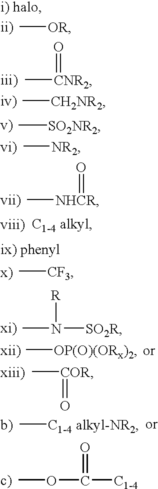
alkyl substituted with one or more of amine or quaternary amine or —OP(O)(ORₓ)₂,
3) heterocycle, such as isochroman, chroman, isothiochroman, thiochroman, benzimidazole, benzothiopyran, oxobenzothiopyran, benzopyran, benzothiopyranylsulfone, benzothiopyranylsulfoxide, the ring or rings being unsubstituted or substituted with one or more of
a) R¹⁴, as defined above,
b) —OC₁₋₄ alkenyl,
c) phenyl-C₁₋₄ alkyl, 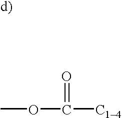
alkyl substituted with one or more of amine or quaternary amine, or —OP(O)(ORₓ)₂, or 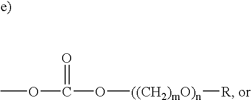
4) A 5 to 7 membered carbocyclic or 7-10 membered bicyclic carbocyclic ring, such as cyclopentane, cyclohexane, indane, norbornane, naphthalene, thiopyran, isothiopyran, or benzopyran, the carbocyclic ring being unsubstituted or substituted with one or more of
a) R¹⁴, as defined above,
b) —CH₂OR,
c) —(CH₂)ₙ—NR₂, C₅₋₁₆alkyl, pyridine, 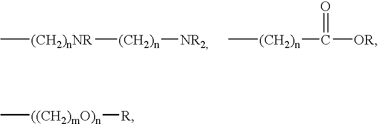
quinuclidiniumyl substituted with R, piperazine-C₁₋₄ alkyl-benzyl substituted one or more with R, or morpholino-C₁₋₄ alkyl-benzyl, 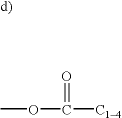
alkyl substituted with one or more of amine or quaternary amine, —OP(ORₓ)₂ or 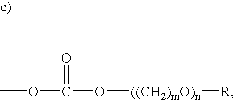
or
f) —C₁₋₄ alkyl-phenyl.

11. (cancelled)

12. A method for inhibiting production of amyloid beta peptide (A beta) in a cell, comprising administering to said cell an effective inhibitory amount of a compound of formula (I): 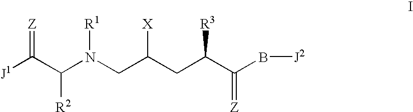
wherein
X is —OH or —NH₂;
Z is —O, —S, or —NH;
R is hydrogen or C₁₋₄ alkyl;
R¹ and R² are independently:
1) hydrogen,
2) —C₁₋₄ alkyl unsubstituted or substituted with one or more of
a) halo,
b) hydroxy,
c) C₁₋₃ alkoxy,
d) aryl unsubstituted or substituted with one or more of C₁₋₄ alkyl, halo, amino, hydroxy or aryl,
e) —W-aryl or —W-benzyl, wherein W is —O—, —S—, or —NH—,
f) a 5-7 membered cycloalkyl group unsubstituted or substituted with one or more of
i) halo,
ii) hydroxy,
iii) C₁₋₃ alkoxy,
iv) aryl,
g) heterocycle unsubstituted or substituted with one or more of hydroxy, oxo, halo, C₁₋₄ alkoxy, C₁₋₄ alkyl optionally substituted with hydroxy; 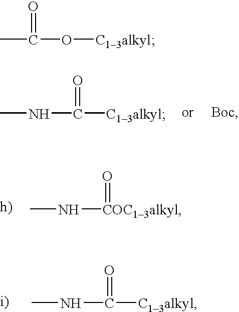
j) —NH—SO₂C₁₋₃alkyl,
k) —NR₂,
l) —COOR, or
m) —((CH₂)ₘO)ₙR wherein m is 2-5 and n is zero, 1, 2 or 3, or
3) aryl, unsubstituted or substituted with one or more of
a) halo,
b) hydroxy,
c) —NO₂ or —NR₂,
d) C₁₋₄alkyl,
e) C₁₋₃alkoxy, unsubstituted or substituted with one or more of —OH or C₁₋₃alkoxy, 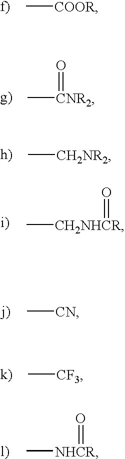
m) aryl C₁₋₃alkoxy,
n) aryl,
o) —NRSO₂R,
P) —OP(O)(ORₓ)₂, or
q) —R⁵, as defined below; or
4) heterocycle unsubstituted or substituted with one or more of hydroxy, oxo, halo, amino, C₁₋₄ alkoxy, C₁₋₄ alkyl optionally substituted with hydroxy; or Boc;
5) carbocyclic unsubstituted or substituted with one or more of halo, amino, hydroxy or C₁₋₄ alkoxy;
R¹ and R² can be joined together to form with the nitrogen to which R¹ is attached a 3 to 10 membered monocyclic or bicyclic saturated ring system which consists of the nitrogen to which R¹ is attached and from 2 to 9 carbon atoms, and is unsubstituted or substituted with
1) hydroxy,
2) C₁₋₄ alkyl unsubstituted or substituted with one or more of
a) halo,
b) hydroxy,
c) C₁₋₃ alkoxy,
d) aryl,
e) a 5-7 membered cycloalkyl group unsubstituted or substituted with one or more of
i) halo,
ii) hydroxy,
iii) C₁₋₃ alkoxy, or
iv) aryl,
f) heterocycle, or
g) —NR²,
3) C₁₋₃ alkoxy, 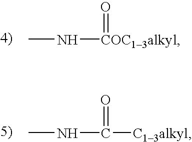
6) —NH—SO₂C₁₋₃ alkyl,
7) heterocycle,
8) —W-aryl, or 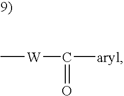
wherein W is defined above; or
R¹ and R² can be joined together to form with the nitrogen to which R¹ is attached a 3 to 10 membered monocyclic or bicyclic saturated ring system which consists of the nitrogen to which R¹ is attached, from 1 to 8 carbon atoms and one or more unsubstituted or substituted heteroatom selected from 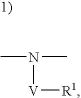
wherein V is absent or 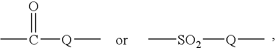
R¹ is defined as above for when R¹ is independent from and not joined to R², and wherein Q is absent or —O—, —NR—, or heterocycle optionally substituted with —C₁₋₄alkyl, 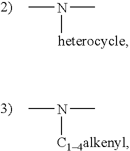
unsubstituted or substituted with aryl, 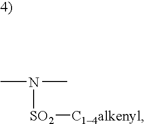
unsubstituted or substituted with aryl, 5) —S(O)p-, wherein p is zero, 1 or 2, or
6) —O—; or
R¹ and R² can be joined together to form with the nitrogen to which R¹ is attached a 3 to 10 membered monocyclic or bicyclic saturated ring system, which consists of the nitrogen to which R¹ is attached and from 2 to 9 carbon atoms, in which the saturated ring system is fused to a phenyl ring and the phenyl ring is unsubstituted or substituted with one or more of
1) halo,
2) C₁₋₃ alkoxy,
3) hydroxy,
4) C₁₋₄ alkyl,
5) —NHR¹, wherein R¹ is defined as above for when R¹ is independent from and not joined to R², or
6) —NH-heterocycle;
R³ is
1) —(CH₂)ᵣ—R⁴, wherein r is zero through 5,
2) C₁₋₄alkenyl-R⁴,
3) C₁₋₄ alkynyl-R⁴;
R⁴ is
1) hydrogen,
2) C₁₋₄ alkyl,
3) C₅-C₁₀ cycloalkyl, optionally substituted with hydroxy,
4) C₆-C₁₀ aryl, unsubstituted or substituted with one or more of
a) halo,
b) hydroxy,
c) —NO₂ or —NR₂,
d) C₁₋₄ alkyl,
e) C₁₋₃ alkoxy, unsubstituted or substituted with one or more of —OH or C₁₋₃ alkoxy, 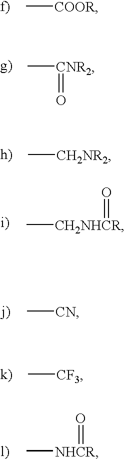
m) aryl C₁₋₃ alkoxy, n) aryl,
o) —NRSO₂R,
p) —OP(O)(ORₓ)₂, or
q) —R⁵, as defined below, or
5) monocyclic or bicyclic heterocyle containing from 1 to 3 heteroatoms chosen from the group consisting of N, O, and S and which is unsubstituted or substituted with R⁵ and optionally with one or more of
a) halo,
b) C₁₋₄ alkyl, or
c) C₁₋₃ alkoxy;
Rₓ is H or aryl;
R⁵ is
1) —W—(CH₂)ₘ—NR⁶R⁷ wherein W is as defined above, m is 2-5, and R⁶ and R⁷ are independently
a) hydrogen,
b) C₁₋₆ alkyl, unsubstituted or substituted with one or more of
i) C₁₋₃ alkoxy,
ii) —OH, or
iii) —NR₂,
c) the same or different and joined together to form a 5-7 member heterocycle, such as morpholino, containing up to two additional heteroatoms selected from 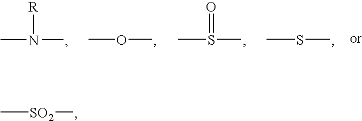
the heterocycle optionally substituted with C₁₋₄ alkyl, or
d) aromatic heterocycle unsubstituted or substituted with one or more of
i) C₁₋₄ alkyl, or
ii) —NR²,
2) —(CH₂)_{q}—NR⁶R⁷ wherein q is 1-5, and R⁶ and R⁷ are defined above, except that R⁶ or R⁷ are not H or unsubstituted C₁₋₆ alkyl, or
3) benzofuryl, indolyl, azacycloalkyl, azabicyclo C₇₋₁₁ cycloalkyl, or benzopiperidinyl, unsubstituted or substituted with C₁₋₄ alkyl;
B is absent, or 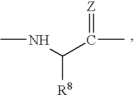
wherein R⁸ is 1) —CH (CH₃)₂,
2) —CH(CH₃)(CH₂CH₃), or
3) -phenyl;
J¹ and J² are independently
1) —YR⁹ wherein Y is —O— or —NH—, and R⁹ is
a) hydrogen,
b) C₁₋₆ alkyl, unsubstituted or substituted with one or more of
i) —NR₂,
ii) —OR,
iii) —NHSO₂C₁₋₄ alkyl,
iv) NHSO₂aryl, or —NHSO₂(dialkylaminoaryl),
v) —CH₂OR,
vi) —C₁₋₄ alkyl, 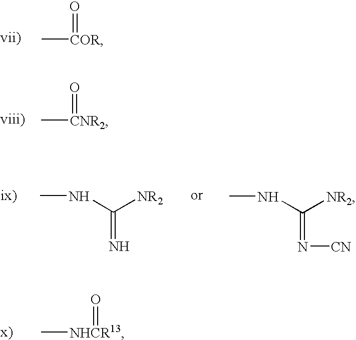
wherein R¹³ is
A) —H
B) —C₁₋₄ alkyl,
C) -aryl,
D) -heterocycle, or
E) —NH—, —O— or —(CH₂)ₙ— wherein n is zero, 1, 2 or 3, substituted with
I) —C₁₋₄ alkyl, unsubstituted or substituted with one or more of aryl or heterocycle, or
II) aryl, unsubstituted or substituted with heterocycle,
xi) —NR₃^{⊕} A⁶³ wherein A^{⊖} is a counterion,
xii) —NR¹⁰R¹¹ wherein R¹⁰ and R¹¹ are the same or different and are C₁₋₅ alkyl joined together directly to form a 5-7 membered heterocycle containing up to one additional heteroatom selected from —O—, —S—, or —NR—,
xiii) aryl,
xiv) —CHO,
xv) —OP(O)ORₓ)₂, 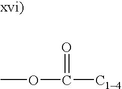
alkyl substituted with one or more of amine or quaternary amine, or —O—((CH₂)ₘO)ₙ—R, or —OP(O)(ORₓ)₂, 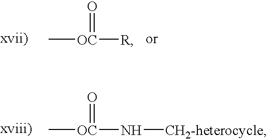
or
c) —((CH₂)ₘO)ₙCH₃ or —((CH₂)ₘO)ₙH, wherein m and n are defined above, or
2) —N(R⁹)ₓ,
3) —NR¹⁰R¹¹ wherein R¹⁰ and R¹¹ are defined above, or 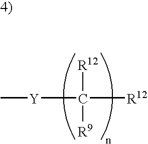
wherein Y, R⁹ and n are defined above; and
R¹² is
1) hydrogen,
2) aryl, unsubstituted or substituted with one or more of
a) R¹⁴, wherein R¹⁴ is 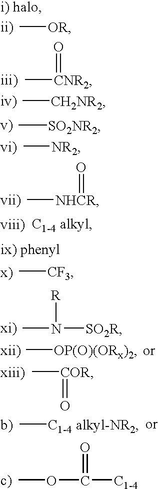
alkyl substituted with one or more of amine or quaternary amine or —OP(O)(ORₓ)₂,
3) heterocycle, such as isochroman, chroman, isothiochroman, thiochroman, benzimidazole, benzothiopyran, oxobenzothiopyran, benzopyran, benzothiopyranylsulfone, benzothiopyranylsulfoxide, the ring or rings being unsubstituted or substituted with one or more of
a) R¹⁴, as defined above,
b) —OC₁₋₄ alkenyl,
c) phenyl-C₁₋₄ alkyl, 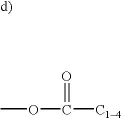
alkyl substituted with one or more of amine or quaternary amine, or —OP(O)(ORₓ)₂, or 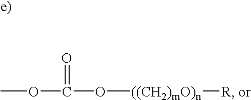
4) A 5 to 7 membered carbocyclic or 7-10 membered bicyclic carbocyclic ring, such as cyclopentane, cyclohexane, indane, norbornane, naphthalene, thiopyran, isothiopyran, or benzopyran, the carbocyclic ring being unsubstituted or substituted with one or more of
a) R¹⁴, as defined above,
b) —CH₂OR,
c) —(CH₂)ₙ—NR₂, C₅₋₁₆alkyl, pyridine, 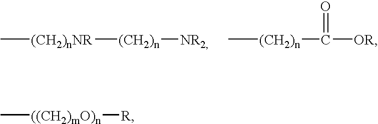
quinuclidiniumyl substituted with R, piperazine-C₁₋₄ alkyl-benzyl substituted one or more with R, or morpholino-C₁₋₄ alkyl-benzyl, 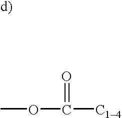
alkyl substituted with one or more of amine or quaternary amine, —OP(ORₓ)₂ or 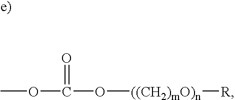
or
f) —C₁₋₄ alkyl-phenyl.

13. The method of claim 12, wherein the cell is an animal cell.

14. The method of claim 13, wherein the animal cell is a mammalian cell.

15. The method of claim 14, wherein the mammalian cell is human.

16. A composition comprising beta-secretase complexed with a compound of formula (I): 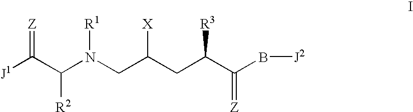
wherein
X is —OH or —NH₂;
Z is —O, —S, or —NH;
R is hydrogen or C₁₋₄ alkyl;
R¹ and R² are independently:
1) hydrogen,
2) —C₁₋₄ alkyl unsubstituted or substituted with one or more of
a) halo,
b) hydroxy,
c) C₁₋₃ alkoxy,
d) aryl unsubstituted or substituted with one or more of C₁₋₄ alkyl, halo, amino, hydroxy or aryl,
e) —W-aryl or —W-benzyl, wherein W is —O—, —S—, or —NH—,
f) a 5-7 membered cycloalkyl group unsubstituted or substituted with one or more of
i) halo,
ii) hydroxy,
iii) C₁₋₃ alkoxy,
iv) aryl,
g) heterocycle unsubstituted or substituted with one or more of hydroxy, oxo, halo, C₁₋₄ alkoxy, C₁₋₄ alkyl optionally substituted with hydroxy; 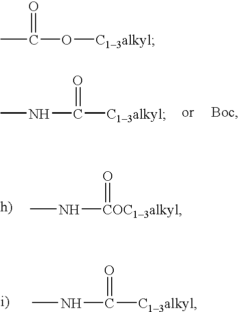
j) —NH—SO₂C₁₋₃alkyl,
k) —NR₂,
l) —COOR, or
m) —((CH₂)ₘO)ₙR wherein m is 2-5 and n is zero, 1, 2 or 3, or
3) aryl, unsubstituted or substituted with one or more of
a) halo,
b) hydroxy,
C) —NO₂ or —NR₂,
d) C₁₋₄alkyl,
e) C₁₋₃alkoxy, unsubstituted or substituted with one or more of —OH or C₁₋₃alkoxy, 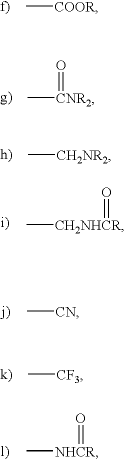
m) aryl C₁₋₃alkoxy,
n) aryl,
o) —NRSO₂R,
p) —OP(O)(ORₓ)₂, or
q) —R⁵, as defined below; or
4) heterocycle unsubstituted or substituted with one or more of hydroxy, oxo, halo, amino, C₁₋₄ alkoxy, C₁₋₄ alkyl optionally substituted with hydroxy; or Boc;
5) carbocyclic unsubstituted or substituted with one or more of halo, amino, hydroxy or C₁₋₄ alkoxy;
R¹ and R² can be joined together to form with the nitrogen to which R¹ is attached a 3 to 10 membered monocyclic or bicyclic saturated ring system which consists of the nitrogen to which R¹ is attached and from 2 to 9 carbon atoms, and is unsubstituted or substituted with
1) hydroxy,
2) C₁₋₄ alkyl unsubstituted or substituted with one or more of
a) halo,
b) hydroxy,
c) C₁₋₃ alkoxy,
d) aryl,
e) a 5-7 membered cycloalkyl group unsubstituted or substituted with one or more of
i) halo,
ii) hydroxy,
iii) C₁₋₃ alkoxy, or
iv) aryl,
f) heterocycle, or
g) —NR₂,
3) C₁₋₃ alkoxy, 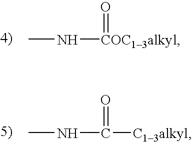
6) —NH—SO₂C₁₋₃ alkyl,
7) heterocycle,
8) —W-aryl, or 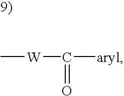
wherein W is defined above; or
R¹ and R² can be joined together to form with the nitrogen to which R¹ is attached a 3 to 10 membered monocyclic or bicyclic saturated ring system which consists of the nitrogen to which R¹ is attached, from 1 to 8 carbon atoms and one or more unsubstituted or substituted heteroatom selected from 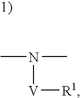
wherein V is absent or 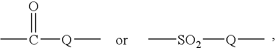
R¹ is defined as above for when R¹ is independent from and not joined to R², and wherein Q is absent or —O—, —NR—, or heterocycle optionally substituted with —C₁₋₄alkyl, 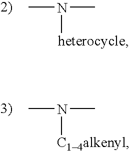
unsubstituted or substituted with aryl, 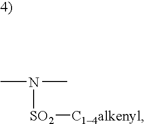
unsubstituted or substituted with aryl, 5) —S(O)p-, wherein p is zero, 1 or 2, or
6) —O—; or
R¹ and R² can be joined together to form with the nitrogen to which R¹ is attached a 3 to 10 membered monocyclic or bicyclic saturated ring system, which consists of the nitrogen to which R¹ is attached and from 2 to 9 carbon atoms, in which the saturated ring system is fused to a phenyl ring and the phenyl ring is unsubstituted or substituted with one or more of
1) halo,
2) C₁₋₃ alkoxy,
3) hydroxy,
4) C₁₋₄ alkyl,
5) —NHR¹, wherein R¹ is defined as above for when R¹ is independent from and not joined to R², or
6) —NH-heterocycle;
R³ is
1) —(CH₂)ᵣ—R⁴, wherein r is zero through 5,
2) C₁₋₄alkenyl-R⁴,
3) C₁₋₄ alkynyl-R⁴;
R⁴ is
1) hydrogen,
2) C₁₋₄ alkyl,
3) C₅-C₁₀ cycloalkyl, optionally substituted with hydroxy,
4) C₆-C₁₀ aryl, unsubstituted or substituted with one or more of
a) halo,
b) hydroxy,
c) —NO₂ or —NR₂,
d) C₁₋₄ alkyl,
e) C₁₋₃ alkoxy, unsubstituted or substituted with one or more of —OH or C₁₋₃ alkoxy, 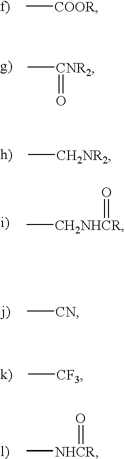
m) aryl C₁₋₃ alkoxy, n) aryl,
o) —NRSO₂R,
p) —OP(O)(ORₓ)₂, or
q) —R⁵, as defined below, or
5) monocyclic or bicyclic heterocyle containing from 1 to 3 heteroatoms chosen from the group consisting of N, O, and S and which is unsubstituted or substituted with R⁵ and optionally with one or more of
a) halo,
b) C₁₋₄ alkyl, or
c) C₁₋₃ alkoxy;
Rₓ is H or aryl;
R⁵ is
1) —W—(CH₂)ₘ—NR⁶R⁷ wherein W is as defined above, m is 2-5, and R⁶ and R⁷ are independently
a) hydrogen,
b) C₁₋₆ alkyl, unsubstituted or substituted with one or more of
i) C₁₋₃ alkoxy,
ii) —OH, or
iii) —NR₂,
c) the same or different and joined together to form a 5-7 member heterocycle, such as morpholino, containing up to two additional heteroatoms selected from 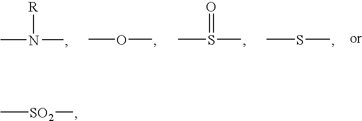
the heterocycle optionally substituted with C₁₋₄ alkyl, or
d) aromatic heterocycle unsubstituted or substituted with one or more of
i) C₁₋₄ alkyl, or
ii) —NR₂,
2) —(CH₂)_{q}—NR⁶R⁷ wherein q is 1-5, and R⁶ and R⁷ are defined above, except that R⁶ or R⁷ are not H or unsubstituted C₁₋₆ alkyl, or
3) benzofuryl, indolyl, azacycloalkyl, azabicyclo C₇₋₁₁ cycloalkyl, or benzopiperidinyl, unsubstituted or substituted with C₁₋₄ alkyl;
B is absent, or 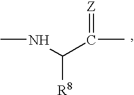
wherein R⁸ is 1) —CH (CH₃)₂,
2) —CH(CH₃)(CH₂CH₃), or
3) -phenyl;
J¹ and J² are independently
1) —YR⁹ wherein Y is —O— or —NH—, and R⁹ is
a) hydrogen,
b) C₁₋₆ alkyl, unsubstituted or substituted with one or more of
i) —NR₂,
ii) —OR,
iii) —NHSO₂C₁₋₄ alkyl,
iv) NHSO₂aryl, or —NHSO₂(dialkylaminoaryl), 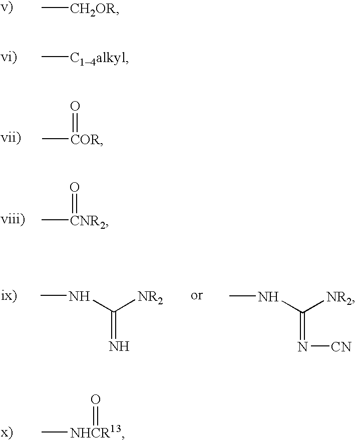
wherein R¹³ is
A) —H
B) —C₁₋₄ alkyl,
C) -aryl,
D) -heterocycle, or
E) —NH—, —O— or —(CH₂)ₙ— wherein n is, zero, 1, 2 or 3, substituted with
I) —C₁₋₄ alkyl, unsubstituted or substituted with one or more of aryl or heterocycle, or
II) aryl, unsubstituted or substituted with heterocycle,
xi) —NR₃^{⊕} A^{⊖} wherein A^{⊖} is a counterion,
xii) —NR¹⁰R¹¹ wherein R¹⁰ and R¹¹ are the same or different and are C₁₋₅ alkyl joined together directly to form a 5-7 membered heterocycle containing up to one additional heteroatom selected from —O—, —S—, or —NR—,
xiii) aryl, 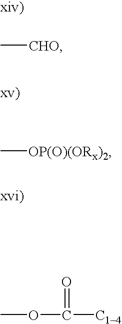
alkyl substituted with one or more of amine or quaternary amine, or —O—((CH₂)ₘO)ₙ—R, or —OP(O)(ORₓ)₂, 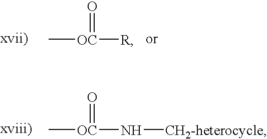
or
c) —((CH₂)ₘO)ₙCH₃ or —((CH₂)ₘO)ₙH, wherein m and n are defined above, or
2) —N(R⁹)ₓ,
3) —NR¹⁰R¹¹ wherein R¹⁰ and R¹¹ are defined above, or 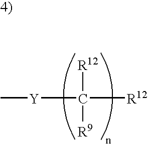
wherein Y, R⁹ and n are defined above; and
R¹² is
1) hydrogen,
2) aryl, unsubstituted or substituted with one or more of
a) R¹⁴, wherein R¹⁴ is 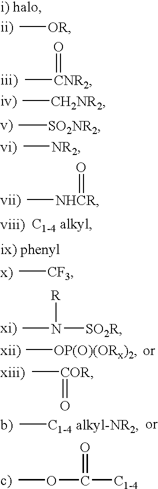
alkyl substituted with one or more of amine or quaternary amine or —OP(O)(ORₓ)₂,
3) heterocycle, such as isochroman, chroman, isothiochroman, thiochroman, benzimidazole, benzothiopyran, oxobenzothiopyran, benzopyran, benzothiopyranylsulfone, benzothiopyranylsulfoxide, the ring or rings being unsubstituted or substituted with one or more of
a) R¹⁴, as defined above,
b) —OC₁₋₄ alkenyl,
c) phenyl-C₁₋₄ alkyl, 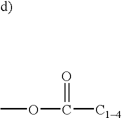
alkyl substituted with one or more of amine or quaternary amine, or —OP(O)(ORₓ)₂, or 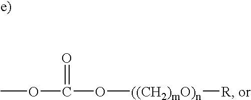
4) A 5 to 7 membered carbocyclic or 7-10 membered bicyclic carbocyclic ring, such as cyclopentane, cyclohexane, indane, norbornane, naphthalene, thiopyran, isothiopyran, or benzopyran, the carbocyclic ring being unsubstituted or substituted with one or more of
a) R¹⁴, as defined above,
b) —CH₂OR,
c) —(CH₂)ₙ—NR₂, C₅₋₁₆alkyl, pyridine, 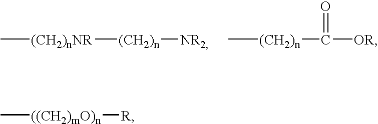
quinuclidiniumyl substituted with R, piperazine-C₁₋₄ alkyl-benzyl substituted one or more with R, or morpholino-C₁₋₄ alkyl-benzyl, 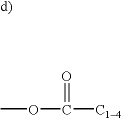
alkyl substituted with one or more of amine or quaternary amine, —OP(ORₓ)₂ or 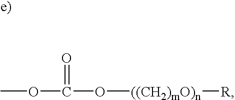
or
f) —C₁₋₄ alkyl-phenyl.

17. A method for producing a beta-secretase complex comprising the composition of claim 16.

18-20. (cancelled)

21. A method of treatment according to claim 5, further comprising administration of one or more therapeutic agents selected from the group consisting of an antioxidant, an anti-inflammatory, a gamma secretase inhibitor, a neurotrophic agent, an acetyl cholinesterase inhibitor, a statin, P-gp inhibitors, an A beta peptide, and an anti-A beta peptide.

22. A method according to claim 5, wherein the compound of Formula (I) is selected from the group consisting of:
N-(2(R)-hydroxy-1(S)-indanyl)-2(R)-phenylmethyl-4(S)-hydroxy-5-(2-(3-(S)-N′-(t-butylcarboxamido)-(4aS,8aS)-decahydroisoquinoline)yl)-pentaneamide,
N-(2(R)-hydroxy-1(S)-indanyl)-2(R)-phenylmethyl(4(S)-hydroxy-5-(1-(4-carbobenzyloxy-2(S)-N′-(t-butylcarboxamido)-piperazinyl))-pentaneamide,
N-(2(R)-hydroxy-1(S)-indanyl)-2(R)-((4-(2-(4-morpholinyl)ethoxy)phenyl)methyl)-4(S)-hydroxy-5-(2-(3(S)-N′-(t-butylcarboxamido)-(4aS,8aS)-decahydroisoquinoline)yl)-pentaneamide,
N-(2(R)-hydroxy-1(S)-indanyl)-2(R)-((4-(2-(4-morpholinyl)ethoxy)phenyl)methyl-4(S)-hydroxy-5-(1-(4-carbobenzyloxy-2(S)-N′-(t-butylcarboxamido)-piperazinyl))-pentaneamide,
N-(2(R)-hydroxy-1(S)-indanyl)-2(R)-((4-((2-hydroxy)-ethoxy)phenyl)methyl)-4(S)-hydroxy-5-(2-(3(S)-N′-(t-butylcarboxamido)-(4aS,8aS)-decahydroisoquinoline)-yl)-pentaneamide,
N-(2(R)-hydroxy-1(S)-indanyl)-2(R)-((4-((2-hydroxy)-ethoxy)phenyl)methyl)-4(S)-hydroxy-5-(1-(4-carbobenzyloxy-2(S)-N′-(t-butylcarboxamido)-piperazinyl))-pentaneamide,
N-(4(S)-3,4-dihydro-1H-2,2-dioxobenzothiopyranyl)-2(R)-phenylmethyl-4(S)-hydroxy-5-(2-(3(S)-N′-(t-butylcarboxamido)-(4aS,8aS)-decahydroisoquinoline)yl)-pentaneamide,
N-(4(S)-3,4-dihydro-1H-2,2-dioxobenzothiopyranyl)-(2(R)-phenylmethyl-4(S)-hydroxy-5-(1-(4-carbobenzyloxy-2(S)-N′-(t-butylcarboxamido)-piperazinyl))-pentaneamide,
N-(2(R)-hydroxy-1(S)-indanyl)-2(R)-phenylmethyl-4(S)-hydroxy-5-(1-(4-(3-pyridylmethyl)-2(S)-N′(t-butylcarboxamido)-piperazinyl))-pentaneamide,
N-(2(R)-hydroxy-1(S)-indanyl)-2(R)-phenylmethyl-4-(S)-hydroxy-5-(1-(N′-(t-butyl)-4(S)-phenoxyprolineamid)yl)-pentaneamide,
N-(2(R)-hydroxy-1(S)-indanyl)-2(R)-phenylmethyl-4-(S)-hydroxy-5-(1-(N′-t-butyl-4(S)-2-naphthyloxy-prolineamid)yl)-pentaneamide,
N-(2(R)-hydroxy-1(S)-indanyl)-2(R)-phenylmethyl-4-(S)-hydroxy-5-(1-(N′-t-butyl-4(S)-1-naphthyloxy-prolineamid)yl)-pentaneamide,
N-(2(R)-hydroxy-1(S)-indanyl)-2(R)-phenylmethyl-4-(S)-amino-5-(2-(3(S)-N′-(t-butylcarboxamido)-(4aS,8aS)-decahydroisoquinoline)yl)-pentaneamide,
N-(2(R)-hydroxy-1(S)-indanyl)-2(R)-phenylmethyl-4-(S)-hydroxy-5-(1-(4-(3-phenylpropionyl)-2(S)-N′-(t-butylcarboxamido)-piperazinyl))-pentaneamide,
N-(2(R)-hydroxy-1(S)-indanyl)-2(R)-phenylmethyl-4-(S)-hydroxy-5-(1-(4-benzoyl-2(S)-N′-(t-butylcarboxamido)-piperazinyl))-pentaneamide,
N-(2(R)-hydroxy-1(S)-indanyl)-2(R)-phenylmethyl-4-(S)-hydroxy-5-(1-(4-(3-phenylpropyl)-2(S)-N′-(t-butylcarboxamido)-piperazinyl))-pentaneamide,
N-(2(R)-hydroxy-1(S)-indanyl)-2(R)-phenylmethyl-4-(S)-amino-5-(1-(4-carbobenzyloxy-2(S)-N′-(t-butylcarboxamido)-piperazinyl))-pentaneamide,
N-(2(R)-hydroxy-1(S)-indanyl)-2(R)-((4-(2-(4-morpholinyl)ethoxy)phenyl)methyl)-4(S)-hydroxy-5-(1-(N′-( t-butyl)-4(S)-phenoxyprolineamid)yl)-pentaneamide,
N-(2(R)-hydroxy-1(S)-indanyl)-2(R)-((4-(2-(4-morpholinyl)ethoxy)phenyl)methyl)-4(S)-hydroxy-5-(1-(N′-t-butyl-4(S)-2-naphthyloxy-prolineamid)yl)-pentaneamide,
N-(2(R)-hydroxy-1(S)-indanyl)-2(R)-((4-(2-(4-morpholinyl)ethoxy)phenyl)methyl)-4(S)-hydroxy-5-(1-(N′-t-butyl-4(S)-1-naphthyloxy-prolineamid)yl)-pentaneamide,
N-(2(R)-hydroxy-1(S)-indanyl)-2(R)-((4-(2-(4-morpholinyl)ethoxy)phenyl)methyl)-4(S)-amino-5-(2-(3(S)-N′-(t-butylcarboxamido)-(4aS,8aS)-decahydroisoquinoline)yl)-pentaneamide,
N-(2(R)-hydroxy-1(S)-indanyl)-2(R)-((4-(2-(4-morpholinyl)ethoxy)phenyl)methyl)-4(S)-hydroxy-5-(1-(4-(3-phenylpropionyl)-2(S)-N′-(t-butylcarboxamido)-piperazinyl))-pentaneamide,
N-(2(R)-hydroxy-1(S)-indanyl)-2(R)-((4-(2-(4-morpholinyl)ethoxy)phenyl)methyl)-4(S)-hydroxy-5-(1-(4-benzoyl-2(S)-N′-(t-butylcarboxamido)-piperazinyl))-pentaneamide,
N-(2(R)-hydroxy-1(S)-indanyl)-2(R)-((4-(2-(4-morpholinyl)ethoxy)phenyl)methyl)-4(S)-hydroxy-5-(1-(4-(3-phenylpropyl)-2(S)-N′-(t-burylcarboxamido))-piperazinyl)-pentaneamide,
N-(2(R)-hydroxy-1(S)-indanyl)-2(R)-((4-(2-(4-morpholinyl)ethoxy)phenyl)methyl)-4(S)-amino-5-(1-(4carbobenzyloxy-2(S)-N′-(t-butylcarboxamido)piperazinyl)-pentaneamide,
N-(2(R)-hydroxy-1(S)-indanyl)-2(R)-((4-((2-hydroxy)-ethoxy)phenyl)methyl)-4(S)-hydroxy-5-(1-(N′-(t-butyl)-4(S)-phenoxyprolineamid)yl)-pentaneamide,
N-(2(R)-hydroxy-1(S)-indanyl)-2(R)-((4-((2-hydroxy)ethoxy)phenyl)methyl)-4(S)-hydroxy-5-(1-(N′-t-butyl-4(S)-2-naphthyloxy-prolineamid)yl)-pentaneamide,
N-(2(R)-hydroxy-1(S)-indanyl)-2(R)-((4-((2-hydroxy)-ethoxy)phenyl)methyl)-4(S)-hydroxy-5-(1-(N′-t-butyl4(S)-1-naphthyloxy-prolineamid)yl)-pentaneamide,
N-(2(R)-hydroxy-1(S)-indanyl)-2(R)-((4-((2-hydroxy)ethoxy)phenyl)methyl)-4(S)-amino-5-(2-(3(S)-N′-(t-butylcarboxamido)-(4aS,8aS)-decahydroisoquinoline)-yl)pentaneamide,
N-(2(R)-hydroxy-1(S)-indanyl)-2(R)-((4-((2-hydroxy)-ethoxy)phenyl)methyl)-4(S)-hydroxy-5-(1-(4-(3-phenylpropionyl)-2(S)-N′-(t-butylcarboxamido)-piperazinyl))pentaneamide,
N-(2(R)-hydroxy-1(S)-indanyl)-2(R)-((4-((2-hydroxy)-ethoxy)phenyl)methyl)-4(S)-hydroxy-5-(1-(4-benzoyl2(S)-N′-(t-butylcarboxamido)-piperazinyl))-pentaneamide,
N-(2(R)-hydroxy-1(S)-indanyl)-2(R)-((4-((2-hydroxy)-ethoxy)phenyl)methyl)-4(S)-hydroxy-5-(1-(4-(3-phenylpropyl)-2(S)-N′-(t-butylcarboxamido))-piperazinyl)-pentaneamide,
N-(2(R)-hydroxy-1(S)-indanyl)-2(R)-((4-((2-hydroxy)-ethoxy)phenyl)methyl)-4(S)-amino-5-(1-(4-carbobenzyloxy-2(S)-N′-(t-butylcarboxamido)-piperazinyl))-pentaneamide,
N-(4(S)-3,4-dihydro-1H-2,2-dioxobenzothiopyranyl)-2(R)-phenylmethyl-4(S)-hydroxy-5-(1-(N′-(t-butyl)-4(S)-phenoxyprolineamid)yl)-pentaneamide,
N-(4(S)-3,4-dihydro-1H-2,2-dioxobenzothiopyranyl)-2-(R)-phenylmethyl-4(S)-hydroxy-5-(1-(N′-t-butyl-4(S)-2-naphthyloxy-prolineamid)yl)-pentaneamide,
N-(4(S)-3,4-dihydro-1H-2,2-dioxobenzothiopyranyl)-2-(R)-phenylmethyl-4(S)-hydroxy-5-(1-(N′-t-butyl-4(S)-1-naphthyloxy-prolineamid)yl)-pentaneamide,
N-(4(S)-3,4-dihydro-1H-2,2-dioxobenzothiopyranyl)-2-(R)-phenylmethyl-4(S)-amino-5-(2-(3(S)-N′-(t-butylcarboxamido)-(4aS,8aS)-decahydroisoquinoline)yl)pentaneamide,
N-(4(S)-3,4-dihydro-1H-2,2-dioxobenzothiopyranyl)-2-(R)-phenylmethyl-4(S)-hydroxy-5-(1-(4-(3-phenylpropionyl)-2(S)-N′-(t-butylcarboxamido)-piperazinyl))pentaneamide,
N-(4(S)-3,4-dihydro-1H-2,2-dioxobenzothiopyranyl)-2-(R)-phenylmethyl-4(S)-hydroxy-5-(1-(4-benzoyl-2(S)-N′-(t-butylcarboxamido)-piperazinyl))-pentaneamide,
N-(4(S)-3,4-dihydro-1H-2,2-dioxobenzothiopyranyl)-2-(R)-phenylmethyl-4(S)-hydroxy-5-(1-(4-(3-phenylpropyl)-2(S)-N′-(t-butylcarboxamido))-piperazinyl)pentaneamide, and
(4(S)-3,4-dihydro-1H-2,2-dioxobenzothiopyranyl)-2-(R)-phenylmethyl-4(S)-amino-5-(1-(4-carbobenzyloxy-2-(S)N′-(t-butylcarboxamido)-piperazinyl))-pentaneamide.

23. A method according to claim 1, where the compound of Formula (I) or a pharmaceutically acceptable salt thereof is administered as a composition comprising one or more pharmaceutically acceptable carriers.
